(19) United States
(12) Patent Application Publication
(10) Pub. No.: US 2024/0425557 A1
(43) Pub. Date: Dec. 26, 2024

(54) Albumin Bound Macromolecule Tri-Agonist Activating GLP-1/GIP/Glucagon Receptors

(71) Applicant: AlbuNext, LLC, El Segundo, CA (US)

(72) Inventors: Patrick Soon-Shiong, Culver City, CA (US); Martin ROBITAILLE, Saint-Colomban (CA); Bing Song, El Segundo, CA (US)

(73) Assignee: AlbuNext, LLC, El Segundo, CA (US)

(21) Appl. No.: 18/753,823

(22) Filed: Jun. 25, 2024

Related U.S. Application Data
(60) Provisional application No. 63/650,095, filed on May 21, 2024.
(60) Provisional application No. 63/523,324, filed on Jun. 26, 2023.
(60) Provisional application No. 63/641,782, filed on May 2, 2024.
(60) Provisional application No. 63/634,379, filed on Apr. 15, 2024.
(60) Provisional application No. 63/631,777, filed on Apr. 30, 2024.
(60) Provisional application No. 63/568,988, filed on Mar. 22, 2024.
(60) Provisional application No. 63/567,402, filed on Mar. 19, 2024.
(60) Provisional application No. 63/566,870, filed on Mar. 18, 2024.
(60) Provisional application No. 63/564,941, filed on Mar. 13, 2024.
(60) Provisional application No. 63/564,426, filed on Mar. 12, 2024.
(60) Provisional application No. 63/561,187, filed on Mar. 4, 2024.
(60) Provisional application No. 63/551,315, filed on Feb. 8, 2024.
(60) Provisional application No. 63/551,334, filed on Feb. 8, 2024.
(60) Provisional application No. 63/551,370, filed on Feb. 8, 2024.
(60) Provisional application No. 63/625,169, filed on Jan. 25, 2024.
(60) Provisional application No. 63/624,692, filed on Jan. 24, 2024.
(60) Provisional application No. 63/619,584, filed on Jan. 10, 2024.

(51) Int. Cl. C07K 14/47; A61K 38/00

(52) U.S. Cl. CPC C07K 14/4722; A61K 38/00; C07K 2319/31

(57) ABSTRACT

A pharmaceutical composition comprises a GPCR agonist fusion protein in which a GPCR agonist peptide is covalently coupled to albumin via a linker in a manner that is resistant to a retro-Michael addition. Advantageously, compositions presented herein avoid decoupling of the agonist form the albumin while retaining the agonist in a steric relationship to the albumin that allows for effective binding and activation of the GPCR while also enabling gp60-mediated transcytosis and FcRn-mediated albumin recycling. These properties enable ultra-low dosages for the GPCR agonist fusion protein to give a therapeutic effect while substantially reducing or even entirely avoiding adverse effects otherwise commonly associated with unbound agonists. Such retro-Michael resistant composition is generally achieved by conformational modification of the albumin, resulting in stereoselective coupling of the linker to the albumin.

This application claims priority to our copending US Provisional patent applications with the Ser. Nos. 63/523,324, filed Jun. 26, 2023, 63/619,584, filed Jan. 10, 2024, 63/624,692, filed Jan. 24, 2024, 63/625,169, filed Jan. 25, 2024, 63/551,315, filed Feb. 8, 2024, 63/551,334, filed Feb. 8, 2024, 63/551,370, filed Feb. 8, 2024, 63/561,187, filed Mar. 4, 2024, 63/564,426, filed Mar. 12, 2024, 63/564,941, filed Mar. 13, 2024, 63/566,870, filed Mar. 18, 2024, 63/567,402, filed Mar. 19, 2024, 63/568,988, filed Mar. 22, 2024, 63/631,777, filed Apr. 9, 2024, 63/634,379, filed Apr. 15, 2024, 63/641,782, filed May 2, 2024, and 63/650,095, filed May 21, 2024, each of which are incorporated by reference herein.

### SEQUENCE LISTING

The content of the XML file of the sequence listing named 1026900063US-C, which is 981,724 bytes in size was created on Jun. 25, 2024, and electronically submitted via Patent Center along with the present application and is incorporated by reference in its entirety.

### FIELD OF THE INVENTION

The field of the invention is pharmaceutical composition and methods, particularly as they relate to agonists, di-agonists, and tri-agonists of glucagon-like peptide 1 (GLP-1) receptors, glucose-dependent insulinotropic polypeptide (GIP) receptors, and/or glucagon (GcG) receptors.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Obesity, a global health crisis, is escalating at an alarming rate. As of 2020, 41.9% of adults in the U.S. were classified as obese. Globally, over 1 billion people, including 650 million adults, are affected by obesity. This condition is associated with a multitude of health complications, including hyperglycemia, type 2 diabetes, hypertension, dyslipidemia, obstructive sleep apnea, atherosclerosis, osteoarthritis, urinary incontinence, non-alcoholic steatohepatitis, cardiovascular diseases, certain cancers, and premature mortality. Furthermore, obesity negatively impacts both physical and mental health, leading to a diminished quality of life and reduced cardiorespiratory fitness, which in turn heightens the risk of cardiovascular diseases and overall mortality.

Similarly, diabetes mellitus has become an increasingly common illness with 537 million adults are affected worldwide in 2021 with a prediction of 783 million by 2045. Of these patients, approximately 90% have type 2 (or non-insulin dependent) diabetes. Often related to obesity, this disease is characterized by increased blood glucose levels, decreased insulin action, and impaired glucose tolerance. Treatment usually includes a diet, weight loss, and exercise regimen as well as the administration of anti-diabetic agents including metformin to decrease hepatic glucose output, sulfonylurea and metformin to increase insulin secretion, and/or thiazolidinediones to enhance insulin sensitivity.

While these drug treatment options are often effective, the disease may progress in spite of treatment, and patients may eventually require daily administration of insulin. Hence, new therapeutic strategies in the form of insulinotropic peptides have been developed for the treatment of Type 2 diabetes, including analogs of peptides that control blood glucose levels, such as glucagon-like peptide 1 (GLP-1), gastric inhibitory polypeptide (GIP), Glucagon, and Amylin.

GLP-1 is a naturally occurring hormone that is released by L cells in the lower intestine in response to nutrient ingestion. In addition to having potent insulinotropic effects, GLP-1 has been shown to suppress glucagon, stimulate pancreatic β-cell proliferation, inhibit gastric emptying, and decrease gastrointestinal (GI) motility, all actions that contribute to the glucose-dependent postprandial maintenance of normal blood glucose levels and regulation of appetite. Because of these pleiotropic effects, GLP-1 analogs make attractive candidates for the treatment of Type 2 diabetes.

One major drawback to the use of native GLP-1 is the extremely short half-life of several minutes, due to cleavage by the ubiquitous proteolytic enzyme dipeptidyl peptidase IV at an N-terminal alanine residue. In contrast to the short-lived GLP-1 peptide, exendin-4 (a 39 amino acid peptide agonist of the glucagon-like peptide 1 (GLP-1) receptor) is resistant to dipeptidyl peptidase IV degradation and has a circulating half-life of approximately 33 minutes in humans. Exendin-4 induces similar physiological effects as GLP-1, through the GLP-1 receptor, and is more potent than native GLP-1. Synthetic exendin-4 (Byetta) has been used as an adjunctive therapy with oral antidiabetic agents in the treatment of non-insulin dependent diabetes mellitus (T2DM). In an approach to further extend half-life of exendin-4, the agonist was encapsulated in poly-(D-L-Lactide-Co-Glycolide) microspheres (Bydureon).

The need for even longer active formulations led to the development of recombinant proteins such as the fusion protein described in CA 2434237 (not marketed) and the fusion protein albiglutide (TANZEUM™, Glaxosmithkline, discontinued), as described in U.S. Pat. No. 7,141,547. Albiglutide is an albumin fusion protein of GLP-1 in which a tandem repeat of Gly8 GLP-1 is directly fused to the N-terminus of human serum albumin. Here, the tandem repeat was intended to improve affinity for the GLP-1 receptor by creating a longer distance between albumin and the distal GLP-1 peptide. While the half-life was extended to 6-8 days, making it suitable for once weekly dosing, the potency of albiglutide was significantly reduced (GLP-1 receptor affinity of albiglutide is 20 nM compared with 0.02 nM for exenatide), most likely due to a combination of the Gly8 modification and the direct covalent fusion with the albumin. When administered as a monotherapy, the decrease in HbAlc from baseline at the 30 mg weekly dose was −0.7% at week 52. At the 50 mg weekly dose, the change from baseline of HbAlc was-0.9%. Despite these high doses of GLP-1 at 30 and 50 mg, the change in HbAlc from baseline never achieved a decrease ≥1%. Based on these findings, it was taught by Knudsen et al (Knudsen L B, Lau J. The Discovery and Development of Liraglutide and Semaglutide. Front Endocrinol (Lausanne). 2019 Apr. 12; 10:155. doi: 10.3389/fendo.2019.00155. PMID: 31031702; PMCID: PMC6474072.) that a GLP-1 agonist, when fused to albumin, will not produce an effective composition, under the premise that GLP-1 agonists could bind to albumin or to the GLP-1 receptor, but not to both. Specifically, the industry taught against irreversible binding to albumin and instead implemented the idea “was to build on reversible binding to albumin as a solution for the systemic protraction of GLP-1 analogs. The main challenge identified in earlier studies was that strong binding to albumin had a negative impact on the potency of compounds for the GLP-1R, due to competition between binding to albumin and binding to the receptor). The theory was that only the free fraction in the plasma that was not bound to albumin would be available to activate the GLP-1R. Therefore, the stronger the affinity to albumin the smaller the free and active circulating fraction of the GLP-1 peptide. This phenomenon had previously been observed with liraglutide analogs, where there was a clear trend for longer fatty acids, with improved affinity for albumin, to be associated with diminished potency for the GLP-1R”.

On the basis of this assumption, a modified approach by the pharmaceutical industry was adopted that made use of reversible non-covalent binding of GLP-1 analogs to albumin. In this approach, GLP-I agonists were acylated to so facilitate non-covalent binding of the acylated GLP-1 agonists to the seven fatty acid binding sites in albumin, resulting in extended half-lives of these compositions. Examples for such approaches include VICTOZA™ (liraglutide, Novo Nordisk, albumin with modified GLP-1 agonist), OZEMPIC™/WEGOVY™ (semaglutide, Novo Nordisk, albumin with modified GLP-1 agonist), MOUNJARO™/ZEPBOUND™ (tirzepatide, Eli Lilly, albumin with modified GLP-1/GIP dual agonist), Pemvidutide (AltImmune, albumin with modified GLP-1/Glucagon dual agonist), efinopegdutide (Merck, albumin with modified GLP-1/Glucagon dual agonist), and survodutide (Zealand, Boehringer, albumin with modified GLP-1/Glucagon dual agonist). While at least some of these formulations provided an increased stability and serum half-life, dissociation of the modified agonist from the albumin carrier it nevertheless required for receptor activation, leading once more to renal clearance of the agonist as well as high doses of GLP-1 since in this reversable approach 99% was bound to albumin and only 1% active material available to ligate with the GLP-1 receptor. Moreover, numerous adverse effects such as nausea, vomiting, and diarrhea are common with formulations having reversible and non-covalent binding of GLP-1 analogs at these high doses. To increase tolerability of such compositions, it is routinely recommended to titrate a subject up to a tolerated therapeutic dosage, which can take several weeks.

In still further known compositions, exendin-4 is covalently bound via a linker to Cys₃₄ of albumin as is described in WO 2007/053946, WO 2009/075859, and WO 2011/109787, and CA 2501421 and CA 2550050 teach further insulinotropic agents with a linker that can be coupled to albumin. While conceptually attractive, similar difficulties as observed with the acylated GLP-1 agonists described above remain, as the covalent bond at the Cys₃₄ of albumin is in such compounds generally subject to a retro-Michael addition reaction and so liberates the formerly bound exendin-4, leading once more to fast renal clearance and potential adverse effects due to the unbound GLP-1 agonist.

Thus, even though various compositions and methods of GPCR agonists, and especially GLP-1, GIP, and glucagon receptor agonists, are known in the art, all or almost all of them suffer from several drawbacks. In particular, small agonist peptides such as GLP-1 are quickly degraded by endopeptidases, while small agonist peptide analogs such as exendin-4 are more stable, but still undergo fast renal clearance. On the other hand, GLP-1 albumin direct fusion proteins have significantly longer serum half-life times, however, are sterically hindered and therefore require very high dosages as shown in Albiglutide. Still further, fatty acid-modified GLP-1 and GLP-1 analogs that are non-covalently bound to hydrophobic pockets in albumin need to dissociate from albumin to activate their target receptor. Unfortunately, dissociated free fatty acid-modified GLP-1 and analogs thereof produce adverse effects once separated from albumin and will also not penetrate the blood brain barrier (BBB). Similarly, where linker-bound exendin-4 is coupled to Cys₃₄ in albumin via Michael addition, free exendin-4 is once more encountered due to a retro-Michael addition reaction, leading once more to fast Exendin-4 clearance and adverse effects due to unbound agonists.

Therefore, there is still a need to provide improved compositions in which a GPCR agonist has high stability in plasma/serum, has agonist activity at low concentrations while remining stably bound to a carrier, and in which the GPCR agonist can readily move from the blood compartment into a target tissue or even pass the blood brain barrier into neural tissues and which can be delivered both subcutaneously, sub dermally, intranasally, and orally.

### SUMMARY OF THE INVENTION

The inventive subject matter is directed to various compositions and methods of GPCR agonists, and particularly insulinotropic agonists, with high stability in plasma/serum, with agonist activity while remaining stably bound to a carrier, and in which the GPCR agonist can readily move from the blood compartment into a target tissue or even pass the blood brain barrier into neural tissues. Advantageously, compositions presented herein are retro-Michael resistant and as such reduce or even entirely avoid adverse effects otherwise encountered with unbound or free agonists.

In one aspect of the inventive subject matter, the inventors contemplate a reaction intermediate that includes (a) isolated and conformationally modified albumin in a solvent, wherein at least one hydrophobic pocket of the albumin is not occupied by a non-covalently bound lipid, and (b) a Michael acceptor reagent.

Most typically, the isolated and conformationally modified albumin is recombinant human albumin, and in certain embodiments the isolated and conformationally modified albumin is a defatted albumin. It is further preferred (but not necessary) that the solvent is an aqueous solvent having a pH of the solvent is pH<7.0. Additionally, it is contemplated that Michael acceptor comprises a planar acceptor group (e.g., a maleimide group or a bromo maleimide group), and in most embodiments, the Michael acceptor reagent further includes a linker that is covalently coupled to a Class B G protein-coupled receptor (GPCR) agonist peptide.

Viewed form a different perspective, the inventors contemplate a chemically modified albumin, wherein the albumin is modified at Cys₃₄ with a coupling group such that a sulfur atom of the Cys₃₄ is covalently bound to a chiral carbon atom of the coupling group, and wherein the chiral atom has a favored stereochemical configuration. For example, the favored stereochemical configuration may be favored at a ratio of at least 70:30, or at a ratio of at least 90:10. As noted above, the albumin is typically human albumin, which may or may not be recombinant. It is further preferred that the albumin is a conformationally modified albumin, and/or that the albumin is in an aqueous solvent having a pH<7.0. Additionally, it is contemplated that the albumin is in an aqueous solvent that further comprises albumin having an unreacted Cys₃₄ group (free albumin), wherein the ratio of the free albumin to the chemically modified albumin is at least 1:1.2. In further contemplated embodiments, the coupling group is covalently coupled to a linker, and the linker may be covalently coupled to a Class B GPCR agonist peptide.

Therefore, it should be appreciated that the inventors also contemplate a chemically modified albumin, wherein the albumin is modified at Cys₃₄ with a Michael addition conjugate, and wherein the Michael addition conjugate is resistant to a retro-Michael addition reaction. Most preferably, the Michael addition conjugate comprises a hydrophilic linker of various lengths, which may be further covalently bound to a Class B GPCR agonist peptide. In still further contemplated embodiments, at least one hydrophobic pocket of the albumin is not occupied by a non-covalently bound lipid. Therefore, the albumin may be an at least partially (or even completely) defatted albumin. Moreover, it is also contemplated that a chiral carbon atom of the Michael addition conjugate forms a covalent bond with sulfur of the Cys₃₄, wherein the chiral carbon atom has a favored stereochemical configuration (e.g., favored stereochemical configuration is favored at a ratio of at least 70:30).

Consequently, the inventors also contemplate a method of modifying albumin at Cys₃₄ that includes a step of providing or preparing a conformationally modified albumin in a solvent, wherein at least one hydrophobic pocket of the conformationally modified albumin is not occupied by a non-covalently bound lipid, and wherein a pH of the solvent is pH<7.0. In a further step, sulfur atom of the Cys₃₄ is then reacted with a coupling group to thereby produce the modified albumin.

In some embodiments, the conformationally modified albumin will be modified by at least partial defatting, and/or the pH of the solvent will be between 4.0 and 6.0. Typically, but not necessarily, the step of reacting is performed at an equimolar ratio between the conformationally modified albumin and the coupling group. As will be appreciated, the modified albumin is retro-Michael resistant with respect to the covalently bound coupling group. With further respect to the step of reacting it is contemplated that the step is at least stereopreferred (e.g., one chiral configuration at least occurs at 60%) or even stereoselective. Most typically, the coupling group will comprise a planar Michael acceptor group (e.g., a maleimide group or a bromo maleimide group), and the coupling group is further covalently coupled to a Class B GPCR agonist peptide.

Viewed from a different perspective, the inventors therefore also contemplate a method of reducing a retro-Michael addition reaction in a Cys₃₄ modified albumin in which the Cys₃₄ is reacted with a Michael addition reagent. Such method will typically include the steps of preparing a conformationally modified albumin in a solvent such that at least one hydrophobic pocket of the conformationally modified albumin is not occupied by a non-covalently bound lipid, wherein the pH of the solvent is pH<7.0; and a step of reacting the sulfur atom of Cys₃₄ with a coupling group of the Michael addition reagent to thereby produce the modified albumin. Most typically, but not necessarily, the conformationally modified albumin is modified by at least partial defatting. It is further preferred that the pH of the solvent is between 4.0 and 6.0.

Additionally, it is contemplated that the step of reacting is performed at an equimolar ratio between the conformationally modified albumin and the coupling group. In contemplated methods, retro-Michael addition reaction is typically reduced by at least 50%. In still further embodiments, it is contemplated that the Michael addition reagent comprises a linker of various lengths that is covalently coupled to a Class B GPCR agonist peptide, and/or that the Michael addition reagent has a structure according to any one of SEQ ID NO: 1-471.

In another aspect of the inventive subject matter, the inventors also contemplate a method of producing a Class B G protein-coupled receptor (GPCR) agonist fusion protein that includes the steps of (a) providing or producing a conformationally modified albumin, and (b) covalently coupling a GPCR agonist peptide to Cys₃₄ of the conformationally modified albumin via a Michael addition reaction, wherein the step of coupling is performed at a pH of pH<7.0.

In at least some embodiments, the conformationally modified albumin is modified by at least partial defatting, and/or the step of coupling is performed with a molar excess of albumin relative to the GPCR agonist peptide. Preferably, but not necessarily, the pH is between 4.0 and 6.0. most typically, the GPCR agonist peptide comprises a linker with a planar Michael acceptor group (e.g., a maleimide group or a bromo maleimide group). In such case, it will be appreciated that the Michael addition reaction produces a stereopreferred or even stereoselective chiral carbon atom in the Michael acceptor group. Moreover, it is contemplated that the GPCR agonist peptide has a structure according to any one of SEQ ID NO: 1-471.

Consequently, the inventors also contemplate a method of producing a retro-Michael addition resistant Class B GPCR fusion protein that includes the steps of (a) providing or producing a conformationally modified albumin, and (b) covalently coupling a GPCR agonist peptide to Cys₃₄ of the conformationally modified albumin via a Michael addition reaction, wherein the step of coupling is performed at a pH of pH<7.0.

Preferably, but not necessarily, the GPCR agonist peptide has a structure according to any one of SEQ ID NO:1-471, and/or the conformationally modified albumin is modified by at least partial defatting. Where desired, it is also contemplated that the step of coupling is performed with a molar excess of albumin to GPCR agonist peptide, and/or that the pH is between 4.0 and 6.0. As noted before, the GPCR agonist peptide may comprise a linker with a planar Michael acceptor group such as a maleimide group or a bromo maleimide group. Therefore, the Michael addition reaction will produce a stereopreferred or even stereoselective chiral carbon atom in the Michael acceptor group.

In yet another aspect of the inventive subject matter, the inventors also contemplate resin-immobilized cysteine-reactive Class B GPCR agonist peptide that includes a GPCR agonist peptide comprising a Lys amino acid to which (a) a linker is covalently bound via an amide bond formed by an epsilon amino group of the Lys amino acid, and (b) a synthetic resin with a spacer is covalently bound via an amide bond formed by a distal amino group in the spacer.

Among other options, especially contemplated linkers of various lengths and amino acid compositions include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA. Moreover, it is generally contemplated that the synthetic resin comprises a polystyrene resin with a tricyclic spacer having the distal amino group (e.g., a Ramage resin), that the substitution level of the resin is approximately 0.4-0.6 mmol/gram, and/or that the synthetic resin is cross-linked with divinylbenzene at about 1%. For example, contemplated GPCR agonist peptides may have a structure according to any one of SEQ ID NO: 1-471.

Therefore, and viewed from a different perspective, the inventors also contemplate a peptide solid state synthesis intermediate that includes a Ramage resin to which a first amino acid of a peptide is covalently coupled at a C-terminus of the peptide via an amide bond, wherein the first amino acid is further covalently coupled to a linker via an amide bond in a side chain of the first amino acid, and wherein the linker comprises a coupling group. Most typically, the first amino acid is lysine, and/or the linker is MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA. It is still further generally preferred that the coupling group comprises a planar Michael acceptor group (e.g., a maleimide group or a bromo maleimide group).

Consequently, the inventors also contemplate a method of synthesizing a peptide containing a linker with a coupling group, wherein the method includes the steps of (a) providing a synthetic resin with a spacer that comprises a distal amino group, (b) using the distal amino group to covalently couple a first amino acid to the resin, wherein the first amino acid has a protected reactive group in a side chain, (c) using solid state synthesis to sequentially couple a plurality of Fmoc-protected amino acids to the first amino acid, thereby obtaining a protected peptide, (d) selectively deprotecting the protected reactive group in the side chain, and reacting the reactive group with a linker, wherein the linker comprises a second protected reactive group, (e) selectively deprotecting the second protected reactive group in the linker, and reacting the second reactive group with a coupling group, and (f) deprotecting the protected peptide and cleaving the deprotected peptide from the resin, thereby forming the peptide containing the linker with the coupling group.

Most typically, the synthetic resin in such method is a Ramage resin, the first amino acid is lysine, and/or the linker is MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA. Likewise, it is typically preferred that the reactive group in the linker comprises a planar Michael acceptor group such as a maleimide group or a bromo maleimide group, and/or that the protected peptide comprises a Class B GPCR agonist peptide.

Therefore, the inventors also contemplate a Class B GPCR agonist peptide with a reactive linker, wherein the GPCR agonist peptide includes a Lys amino acid to which the linker is covalently bound via an amide bond formed by an epsilon amino group of the Lys amino acid, wherein the linker further comprises a thiol reactive group, and wherein the GPCR agonist peptide has a structure according to any one of SEQ ID NO: 1-471.

As noted above, it is contemplated that the thiol reactive group comprises a planar Michael acceptor group such as a maleimide group or a bromo maleimide group, and/or that the linker is selected from the group consisting of MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA.

In a still further aspect of the inventive subject matter, the inventor also contemplates Class B GPCR agonist peptide with a reactive linker that includes (a) a GPCR agonist peptide comprising a Lys amino acid, and (b) a linker covalently bound to the GPCR agonist peptide via an amide bond formed by an epsilon amino group of the Lys amino acid and a carboxyl group of the linker, and wherein the linker further comprises a maleimide group, wherein the GPCR agonist peptide has a structure according to any one of SEQ ID NO:1-471. The linker in such agonist peptides is preferably MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, or (AEEEA) 2-OA-(bromo) MPA.

In view of the above, the inventors therefore also contemplate a Class B GPCR agonist fusion protein that includes (a) a conformationally modified albumin comprising a Cys₃₄ amino acid, (b) a GPCR agonist peptide comprising a Lys amino acid, and (c) a linker covalently coupling the conformationally modified albumin to the GPCR agonist peptide, wherein the linker is covalently bound to the GPCR agonist peptide via an amide bond formed by an epsilon amino group of the Lys amino acid, and wherein the linker is covalently bound to the albumin via a covalent bond between a chiral carbon atom of a coupling group in the linker and a sulfur atom in the Cys₃₄ amino acid. Preferably, but not necessarily, the conformationally modified albumin is an at least partially defatted albumin, and/or the chiral carbon atom has a stereopreferred configuration. Most typically, the linker is retro-Michael resistant.

In particularly preferred embodiments, the fusion protein is retro-Michael resistant albenatide (AB-013-AEEA-succinimide (SEQ ID NO:2)-albumin), retro-Michael resistant albenatidex (AB-014-AEEA-succinimide (SEQ ID NO:185)-albumin), retro-Michael resistant albugiptide (AB-029-AEEA-succinimide (SEQ ID NO:7)-albumin), or retro-Michael resistant albutide (AB-044-AEEA-succinimide (SEQ ID NO:223)-albumin).

In yet another aspect of the inventive subject matter, the inventor also contemplates a pharmaceutical composition that includes a Class B GPCR agonist fusion protein in combination with an aqueous carrier, wherein the GPCR fusion protein comprises (a) a conformationally modified albumin comprising a Cys₃₄ amino acid, (b) a GPCR agonist peptide comprising a Lys amino acid, and (c) and a linker covalently coupling the conformationally modified albumin to the GPCR agonist peptide, wherein the linker is covalently bound to the GPCR agonist peptide via an amide bond formed by an epsilon amino group of the Lys amino acid, and wherein the linker is covalently bound to the albumin via a covalent bond between a chiral carbon atom of a coupling group in the linker and a sulfur atom in the Cys₃₄ amino acid. Most typically, the aqueous carrier has a pH of pH<7.0 (e.g., between 4.0 and 6.0).

In such composition it is generally preferred that the GPCR agonist peptide and linker has a structure according to any one of SEQ ID NO: 1-471. Viewed from a different perspective, it is contemplated that the linker is MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA. Where desired, it is further contemplated that the composition further comprises albumin with an unreacted Cys₃₄ amino acid. Additionally, it is contemplated that the composition may be formulated for injection or intranasal administration. Moreover, in at least some embodiments the composition comprises the GPCR agonist peptide at a concentration of equal or less than 1.5 mg/mL, and/or the composition further comprises metformin and/or a thiazolidinedione.

In particularly preferred compositions, the GPCR agonist fusion protein is retro-Michael resistant albenatide (AB-013-AEEA-succinimide (SEQ ID NO:2)-albumin), retro-Michael resistant albenatidex (AB-014-AEEA-succinimide (SEQ ID NO:185)-albumin), retro-Michael resistant albugiptide (AB-029-AEEA-succinimide (SEQ ID NO:7)-albumin), or retro-Michael resistant albutide (AB-044-AEEA-succinimide (SEQ ID NO:223)-albumin).

Viewed from a different perspective, the inventors contemplate a pharmaceutical composition that comprises a Class B GPCR agonist fusion protein in combination with an aqueous carrier, wherein the GPCR fusion protein is a Michael addition product of a conformationally modified albumin and linker that is further covalently bound to a GPCR agonist peptide, and wherein the fusion protein is retro-Michael addition resistant. Most typically, the composition is formulated for injection or intranasal administration, the composition comprises the GPCR agonist peptide at a concentration of equal or less than 1.5 mg/mL, and/or the formulation has a pH of between 4.0 and 6.0.

Where desired, it is contemplated that the composition further comprises albumin with an unreacted Cys₃₄ amino acid. In addition, it is preferred that the Michael addition product is a stereopreferred or stereoselective Michael addition product and/or that the GPCR agonist peptide and linker has a structure according to any one of SEQ ID NO:1-471.

Therefore, the inventors also contemplate a pharmaceutical composition that includes a Class B GPCR agonist fusion protein in combination with an aqueous carrier, wherein the GPCR fusion protein is a Michael addition product of an albumin and linker that is further covalently bound to a GPCR agonist peptide, wherein the composition further comprises albumin with an unreacted Cys₃₄ amino acid, and wherein the aqueous carrier has a pH of pH<7.0. For example, the ratio of the albumin with the unreacted Cys₃₄ amino acid and the albumin to which the linker and GPCR agonist peptide is coupled is at least 0.3:1.0. Moreover, the albumin with the unreacted Cys₃₄ amino acid and the albumin to which the linker and GPCR agonist peptide is coupled are recombinant human albumin. In at least some embodiments, the albumin with the unreacted Cys₃₄ amino acid is a conformationally modified albumin, and/or the GPCR agonist peptide and linker has a structure according to any one of SEQ ID NO:1-471.

Viewed from a different perspective, the inventors also contemplate a pharmaceutical composition that comprises a pharmaceutically acceptable liquid carrier in combination with a Class B G protein-coupled receptor (GPCR) agonist fusion protein in which a GPCR agonist peptide is covalently coupled to albumin via a linker. A pharmaceutically accepted liquid carrier is contemplated which is safe and can accommodate sufficient concentration of the agonist and enables the delivery of the GPCR agonist fusion protein in the form of a subcutaneous, intradermal, intranasal, intrathecal and oral delivery to allow sufficient systemic, mucosal or neural absorption. In such composition, the pharmaceutically acceptable liquid carrier is substantially free of unbound GPCR agonist peptide. Among other suitable GPCR agonist fusion proteins, especially preferred GPCR agonist fusion proteins include retro-Michael resistant albenatide (AB-013-AEEA-succinimide (SEQ ID NO:2)-albumin), retro-Michael resistant albenatidex (AB-014-AEEA-succinimide (SEQ ID NO:185)-albumin), retro-Michael resistant albugiptide (AB-029-AEEA-succinimide (SEQ ID NO:7)-albumin), or retro-Michael resistant albutide (AB-044-AEEA-succinimide (SEQ ID NO:223)-albumin). Most typically, the quantity of unbound GPCR agonist peptide is equal or less than 1%, or equal or less than 0.1% of a quantity of the GPCR agonist peptide that is covalently coupled to albumin via the linker.

In yet additional aspects of the inventive subject matter, the inventors contemplate a method of making a pharmaceutical composition that includes the steps of (a) providing a pharmaceutically acceptable carrier having a pH that is pH<7.0, and (b) including into the pharmaceutically acceptable carrier a retro-Michael resistant G protein-coupled receptor (GPCR) agonist fusion protein to which a GPCR agonist peptide is covalently bound, wherein the GPCR agonist peptide is present in the composition at a concentration of equal or less than 1.5 mg/mL. Among other suitable routes of administration, it is generally preferred that the composition is formulated for injection or intranasal administration. A pharmaceutically accepted liquid carrier is contemplated which is safe and can accommodate sufficient concentration of the agonist and enables the delivery of the GPCR agonist fusion protein in the form of a subcutaneous, intradermal, intranasal, intrathecal and oral delivery to allow sufficient systemic, mucosal or neural absorption.

In especially preferred methods, the GPCR agonist fusion protein is retro-Michael resistant albenatide (AB-013-AEEA-succinimide (SEQ ID NO:2)-albumin), retro-Michael resistant albenatidex (AB-014-AEEA-succinimide (SEQ ID NO:185)-albumin), retro-Michael resistant albugiptide (AB-029-AEEA-succinimide (SEQ ID NO:7)-albumin), or retro-Michael resistant albutide (AB-044-AEEA-succinimide (SEQ ID NO:223)-albumin).

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 shows a schematic illustration of the structure for retro-Michael resistant albenatide in which the peptide sequence surrounding Cys₃₄ is emphasized.

FIG. 2 shows a schematic illustration of the structure for retro-Michael resistant albenatide.

FIG. 3 shows a schematic overall structure of the retro-Michael resistant albenatide in which the agonist peptide portion has bound to a GLP-1 receptor.

FIG. 4 is an exemplary collection of GLP-1 agonist peptides suitable for use herein.

FIG. 5 is an exemplary collection of GLP-1/GIP dual agonist peptides suitable for use herein.

FIG. 6 is an exemplary collection of GLP-1/GcG dual agonist peptides suitable for use herein.

FIG. 7 is an exemplary collection of GLP-1/GIP/GcG tri-agonist peptides suitable for use herein.

FIG. 8 is an exemplary collection of further GLP-1/GIP/GcG tri-agonist peptides suitable for use herein.

FIG. 9 is an exemplary collection of still further GLP-1/GIP/GcG tri-agonist peptides suitable for use herein.

FIG. 10 is an exemplary collection of yet further GLP-1/GIP/GcG tri-agonist peptides suitable for use herein.

FIG. 11 is an exemplary collection of additional GLP-1/GIP/GcG tri-agonist peptides suitable for use herein.

FIG. 12 is an exemplary collection of also contemplated agonist peptide with exemplary linker structures.

FIG. 13 is one exemplary generic sequence for tri-agonist peptides suitable for use herein.

FIG. 14 is another exemplary generic sequence for tri-agonist peptides suitable for use herein.

FIG. 15 depicts exemplary results for binding energy of the tested dual-agonist peptides with the GIP receptor and GLP-1 receptor.

FIG. 16 depicts calculated self-energies for the dual agonists using tirzepatide as control.

FIG. 17 depicts exemplary results for tirzepatide and GLP-1 and GIP receptors.

FIG. 18 depicts exemplary results for AB-804-1 and GIP receptors.

FIG. 19 depicts exemplary results for AB-804-1 and GLP-1 receptors.

FIG. 20 depicts exemplary results for binding interactions and interaction pair counts for AB-804-2 and GIP receptors.

FIG. 21 depicts exemplary results for binding interactions and interaction pair counts for AB-804-2 and GLP-1 receptors.

FIG. 22 depicts exemplary results for binding interactions and interaction pair counts for AB-804-3 and GIP receptors.

FIG. 23 depicts exemplary results for binding interactions and interaction pair counts for AB-804-3 and GLP-1 receptors.

FIG. 24 depicts comparative results for pair interaction energy for native GLP-1, GIP, and GcG complexes.

FIG. 25 depicts a selection of exemplary linkers suitable for use herein.

FIG. 26 shows a schematic overall structure of human albumin.

FIG. 27 illustrates a schematic side view of the Cys₃₄ hydrophobic pocket in conformationally modified human albumin.

FIG. 28A shows a schematic view of the MPA coupling group entering the hydrophobic pocket of conformationally unmodified human albumin of FIG. 27, with the arrows indicating the indiscriminate direction of attack of the sulfur atom relative to the planar maleimide group of the MPA.

FIG. 28B shows a schematic view of the MPA coupling group entering the hydrophobic pocket of conformationally modified human albumin of FIG. 27 with the arrow indicating the stereopreferred or stereoselective direction of attack of the sulfur atom relative to the planar maleimide group of the MPA.

FIG. 28C shows a schematic superposition of conformationally unmodified human albumin (dark gray) and modified human albumin (light gray) with the arrows indicating the position of the sulfur atom in Cys₃₄.

FIG. 29 is a graph depicting exemplary pharmacokinetic data for conformationally unmodified AB-013.

FIG. 30 is a graph depicting exemplary results from serum of the experiment in FIG. 29 when observed on a PAGE gel.

FIG. 31 depicts exemplary structures of peptide agonist intermediates bound to a Ramage or Rink resin.

FIG. 32 depicts generically agonist peptide intermediates.

FIG. 33 depicts the efficacy Albenatide in inducing weight loss in diet induced obese (DIO) mouse models demonstrating equivalence to semaglutide.

FIG. 34 depicts the food and water consumption following Albenatide compared to semaglutide.

FIG. 35 depicts the efficacy of Albenatide in glucose control in DIO mice demonstrating that covalently bound GLP-1 agonists react with GLP-1 receptor and has a biological efficacy effect, contrary to the teaching of current formulations.

FIG. 36 depicts the efficacy of Albenatide in reducing fatty livers (NASH) with histological evidence of control and semaglutide as comparators demonstrating that covalently bound GLP-1 agonists react with GLP-1 receptor and has a biological efficacy effect, contrary to the teaching of current formulations.

FIG. 37A-D depicts liver function and cholesterol improvements in the DIO mouse model following Albenatide demonstrating that covalently bound GLP-1 agonists react with GLP-1 receptor and has a biological efficacy effect, contrary to the teaching of current formulations.

FIG. 38 depicts anti-inflammatory improvement with cytokines following Albenatide

### DETAILED DESCRIPTION

The inventors have now discovered that various GPCR agonist peptide fusion proteins can be prepared that exhibit high stability in plasma/serum, that have agonist activity at low concentrations while remining stably bound to a carrier, and that can readily move from the blood compartment into a target tissue and even pass the blood brain barrier. Such fusion proteins are prepared by use of a linker that irreversibly and covalently connects the albumin to a Class B GPCR agonist peptide (such as an incretin or insulinotropic agent or analog thereof). In this context, it should be appreciated that the term ‘fusion protein’ in conjunction with the compounds presented herein includes proteins in which a first peptide portion (e.g., agonist peptide) is covalently bound to a second peptide portion (e.g., albumin) via a linker moiety, where the linker may be a third peptide portion or a non-peptide portion (e.g., AEEA-MPA). Thus, the term ‘fusion protein’ in conjunction with the compounds presented herein may also be referred to as ‘bioconjugate’ or ‘conjugate’.

For example, in some preferred embodiments, one end of the linker will be covalently bound to a reactive group at the C-terminus of the agonist peptide (e.g., via an amide bond) while the other end of the linker is covalently bound to the albumin at Cys₃₄ via a retro-Michael resistant thioether bond in which the carbon atom of the coupling group in the linker has a stereospecific configuration that renders the bond retro-Michael resistant within the context of the hydrophobic pocket in which the Cys₃₄ is located. In other preferred embodiments, one end of the linker will be covalently bound to a reactive group in a side chain of an amino acid at an intermediate position in the agonist peptide (e.g., via an amide bond) and the other end of the linker will be covalently bound to the albumin at Cys₃₄ via a retro-Michael resistant thioether bond in which the carbon atom of the coupling group in the linker has a stereospecific configuration that renders the bond retro-Michael resistant within the context of the hydrophobic pocket in which the Cys₃₄ is located. Moreover, suitable linkers will provide a rotational degree of freedom and a steric distance between the agonist peptide and the albumin so as to enable binding of the agonist peptide to the target receptor and activation of the target receptor while remining covalently bound to the albumin.

Advantageously, contemplated retro-Michael resistant albumin-bound insulinotropic agonists will transcytose rapidly to the tissue microenvironment of the pancreas and the brain, leaving the plasma compartment through the gp60/caveolin-1/caveoli pathway with long-acting agonist via FcRn rescue. As such, contemplated compounds and compositions can take advantage of cell mediated transport, thereby allowing for ultra-low dose and ultra-low plasma concentration to so enable a therapeutic effect with a high therapeutic index and a substantially lower GI adverse event profile.

As will be appreciated, especially contemplated peptide agonists suitable for use in conjunction with the teachings presented herein will particularly include GPCR agonists (and especially Class B) and all derivatives, analogs, and fragments thereof. Therefore, and among other contemplated peptide agonists, GLP-1, GIP, Glucagon (GcG), Amylin, NPY2, Neuropeptide Y, and PYY, and derivatives, analogs, and fragments are especially preferred. It should furthermore be appreciated that the peptide agonists contemplated herein may have binding and activation specificity towards a single receptor (mono-agonist), two receptors (dual-agonist), or three receptors (tri-agonist). In this context, it should be particularly appreciated that contrary to conventional wisdom, compounds can be produced in which the ligand is covalently and irreversibly bound to the albumin, and in which the same ligand can bind to and activate two, or even three distinct G-protein coupled receptors (GLP-1, GIP, and/or GcG receptors). Consequently, it should be recognized that the biological activity of fusion proteins containing such peptide agonists may be tailored towards specific uses (e.g., insulinotropic, appetite suppressant, etc.).

Viewed from a different perspective, and as shown in more detail below, GPCR (and especially Class B GPCR) agonist fusion proteins with high stability, high receptor binding, and no peptide loss can be prepared that activate GLP-1, GIP, GcG, NPY2, Y2, or PYY as mono-agonists, or that activate GLP-1/GIP, GLP-1/GcG, GLP-1/NPY2, GLP-1/Y2, or GLP-1/PYY as di-agonists, or that activate GLP-1/GIP/GcG as tri-agonists. Moreover, as such agonist peptides are irreversibly covalently bound to conformationally modified albumin, gp60-mediated transcytosis and FcRn albumin recycling will allow these fusion proteins to cross the blood brain barrier and to achieve high half-life times while being quickly moved from the blood compartment to a target tissue compartment (e.g., neural tissue, hepatic tissue, adipose tissue, and/or pancreatic tissue).

In at least some embodiments, it is preferred that the peptide agonist will be exendin-4 or an exendin-4 derivative that includes a modified amino acid to avoid peptide cleavage by dipeptidyl peptidase-4, and that includes a C-terminal tryptophan cage (‘fish-hook’) structure to further enhance stability. Moreover, and depending on the particular amino acid substitutions (relative to exendin-4), contemplated peptide agonists will exhibit equal or higher affinity than native GLP-1 to the GLP-1 receptor, resulting in higher potency. Additionally, one or more amino acid substitutions (relative to exendin-4) will also enable multi-agonist activity (e.g., tri-agonist activity towards GLP-1/GIP/Glucagon receptors).

In one exemplary fusion protein, retro-Michael resistant albenatide (acting as a GLP-1 agonist) comprises exendin-4 (SEQ ID NO:3) to which on the C-terminus a modified lysine is covalently coupled, wherein the modified lysine contains a linker with a coupling group. In this example, the modified lysine forms a peptide bond with the C-terminal serine of exendin-4, and the modified lysine further includes an AEEA-MPA group that is covalently bound to the epsilon amino group of the lysine via an amide bond. Upon reaction of the MPA coupling group with the thiol group of Cys₃₄ in the albumin in a stereopreferred or stereoselective manner as described in more detail below, the retro-Michael resistant albenatide (AB-013-AEEA-succinimide-albumin). Of course, it should be appreciated that various alternative linker moieties may be used, and suitable alternative linkers with coupling groups include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, or (AEEEA) 2-OA-(bromo) MPA.

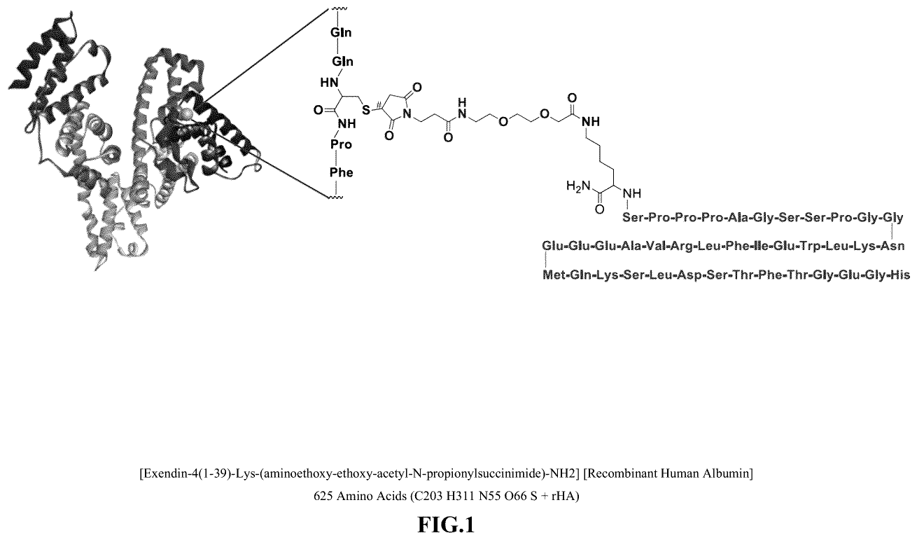

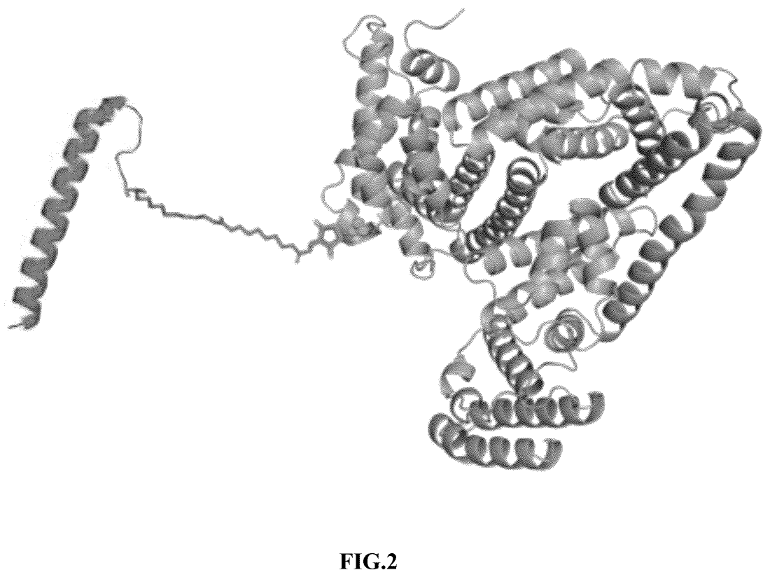

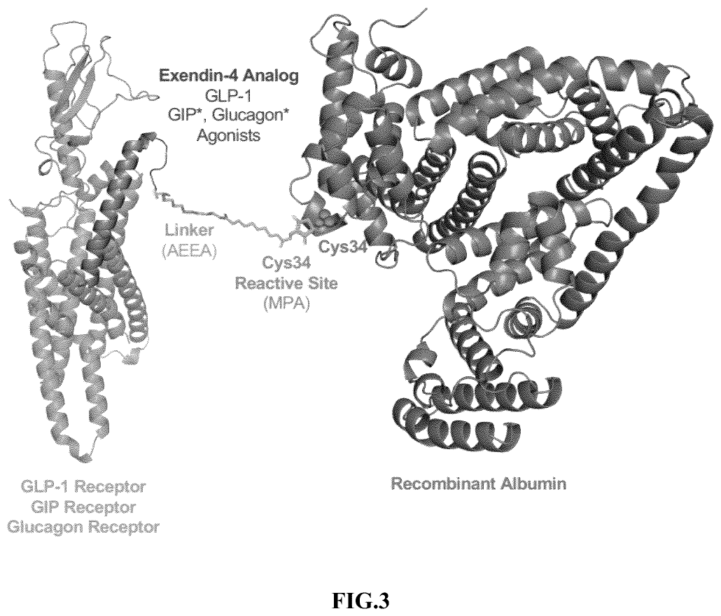

The retro-Michael resistant albenatide (AB-013-AEEA-succinimide (SEQ ID NO:2)-albumin) is exemplarily depicted in FIG. 1 in which the peptide sequence surrounding Cys₃₄ is emphasized and in which the #sign denotes the non-racemic chiral atom, and FIG. 2 exemplarily depicts the overall structure of the retro-Michael resistant albenatide of FIG. 1 above. FIG. 3 shows the overall structure of the retro-Michael resistant albenatide in which the agonist peptide has bound to a GLP-1 receptor (note that the GIP receptor and GcG receptor are structurally highly similar to the GLP-1 receptor, and as such FIG. 3 is also representative of these agonist receptor complexes). As will be readily recognized from FIG. 3, the linker affords sufficient flexibility and steric distance between the covalently bound agonist peptide and the target receptor such as to allow effective binding and activation of the receptor while the agonist remains covalently bound to the albumin.

In another exemplary fusion protein, and following the same approach, a retro-Michael resistant albenatidex (AB-014-AEEA-succinimide (SEQ ID NO:185)-albumin) (acting as a GLP-1 agonist) comprises a chimeric GLP-1/exendin-4 portion to which on the C-terminus a modified lysine is covalently coupled, wherein the modified lysine contains a linker with a coupling group. In this example, the modified lysine forms a peptide bond with the C-terminal serine of chimeric peptide, and the modified lysine further includes an AEEA-MPA group that is covalently bound to the epsilon amino group of the lysine via an amide bond as is shown in SEQ ID NO.185. Upon reaction of the MPA coupling group with the thiol group of Cys₃₄ in the albumin in a stereopreferred or stereoselective manner as described in more detail below, the retro-Michael resistant albenatidex is formed (AB-014-AEEA-succinimide-albumin). As noted above, it should be appreciated that various alternative linker moieties may be used, and suitable alternative linkers with coupling groups include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, or (AEEEA) 2-OA-(bromo) MPA.

In still another exemplary fusion protein, and once more following the same approach, a retro-Michael resistant albugiptide (AB-029-AEEA-succinimide (SEQ ID NO:7)-albumin) (acting as a GIP/GLP-1 dual agonist) comprises a modified exendin-4 portion to which on the C-terminus a modified lysine is covalently coupled, wherein the modified lysine contains a linker with a coupling group. In this example, the modified lysine forms a peptide bond with the C-terminal serine of the modified exendin-4, and the modified lysine further includes an AEEA-MPA group that is covalently bound to the epsilon amino group of the lysine via an amide bond as is shown in SEQ ID NO:7. Once more, upon reaction of the MPA coupling group with the thiol group of Cys₃₄ in the albumin in a stereopreferred or stereoselective manner as described in more detail below, the retro-Michael resistant albugiptide is formed (AB-029-AEEA-succinimide-albumin). As already noted above, it should be appreciated that various alternative linker moieties may be used, and suitable alternative linkers with coupling groups include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, or (AEEEA) 2-OA-(bromo) MPA.

In a still further exemplary fusion protein, and yet again following the same approach, a retro-Michael resistant albutide (AB-044-AEEA-succinimide (SEQ ID NO:223)-albumin) (acting as a GIP/GLP-1/GcG tri-agonist) comprises a modified exendin-4 portion to which on the C-terminus a modified lysine is covalently coupled, wherein the modified lysine contains a linker with a coupling group. In this example, the modified lysine forms a peptide bond with the C-terminal serine of the modified exendin-4, and the modified lysine further includes an AEEA-MPA group that is covalently bound to the epsilon amino group of the lysine via an amide bond as is shown in SEQ ID NO:223. Once more, upon reaction of the MPA coupling group with the thiol group of Cys₃₄ in the albumin in a stereopreferred or stereoselective manner as described in more detail below, the retro-Michael resistant albutide (AB-044-AEEA-succinimide (SEQ ID NO:223)-albumin) is formed. Additionally, a closely related retro-Michael resistant fusion protein is particularly contemplated, AB-045-AEEA-succinimide (SEQ ID NO:69)-albumin. Once more, it should be appreciated that various alternative linker moieties may be used, and suitable alternative linkers with coupling groups include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA.

Based on the above examples, it should therefore be appreciated that numerous retro-Michael resistant fusion proteins with albumin can be prepared that avoid the shortcomings of fusion proteins in which the agonist peptide can deconjugate. Indeed, while numerous Class B GPCR agonist peptides are particularly contemplated herein, a large variety of alternative drug conjugates are also deemed suitable and include various peptide hormones, cytokines, chemokines, and other biologically active peptides and even larger proteins. In each of these fusion proteins, it is contemplated that the same or similar linkers can be used as discussed in more detail below, and that the fusion protein, once prepared, will have retro-Michael resistant properties.

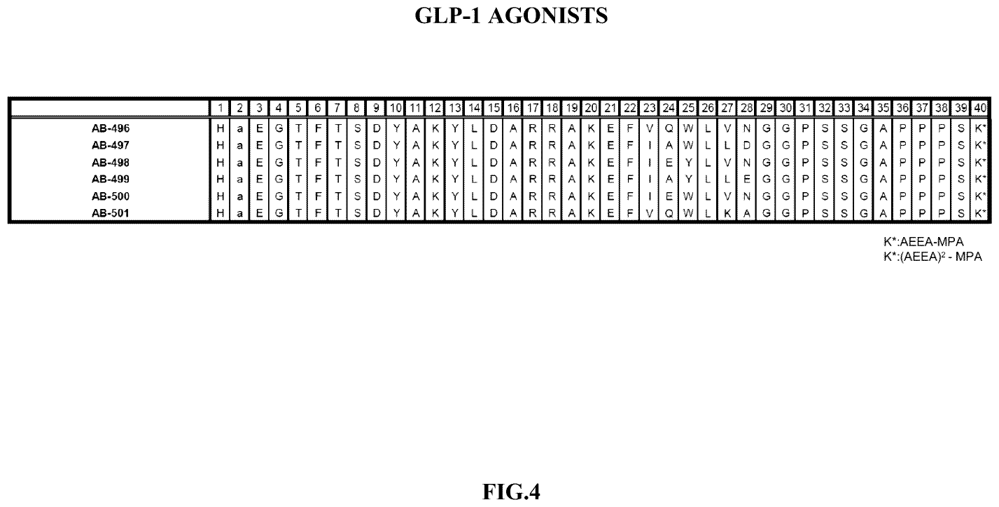

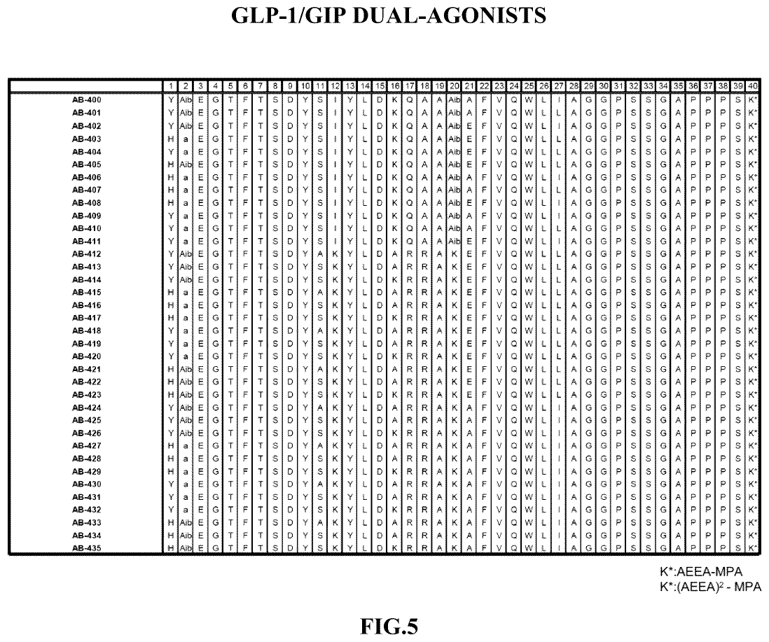

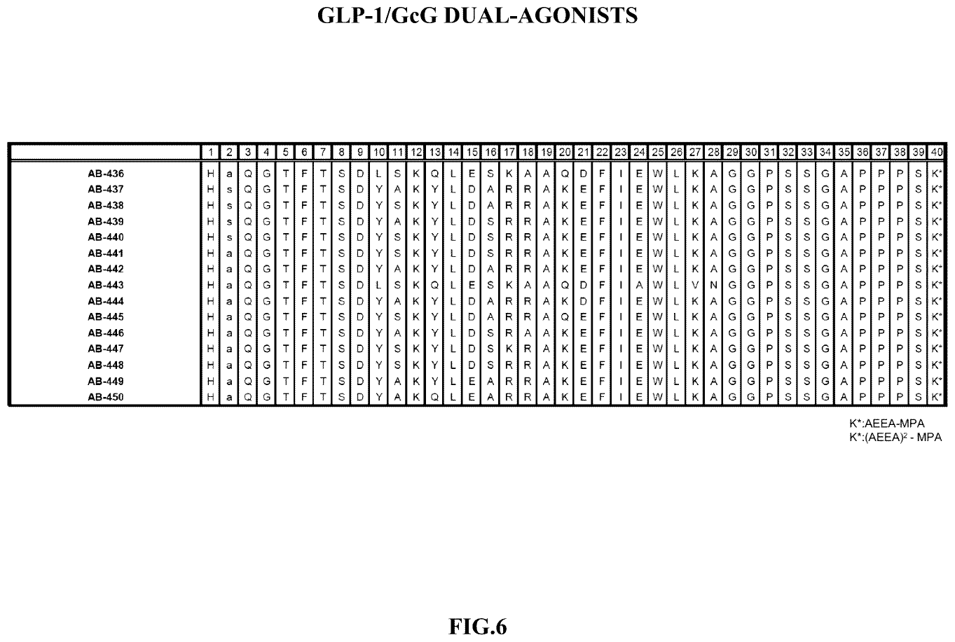

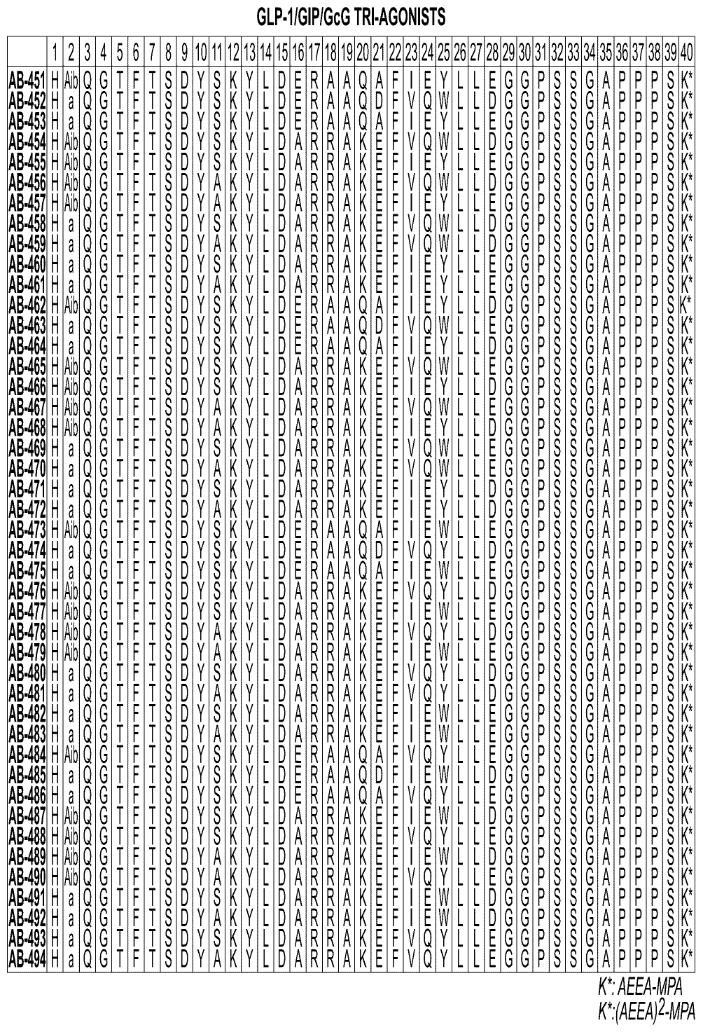

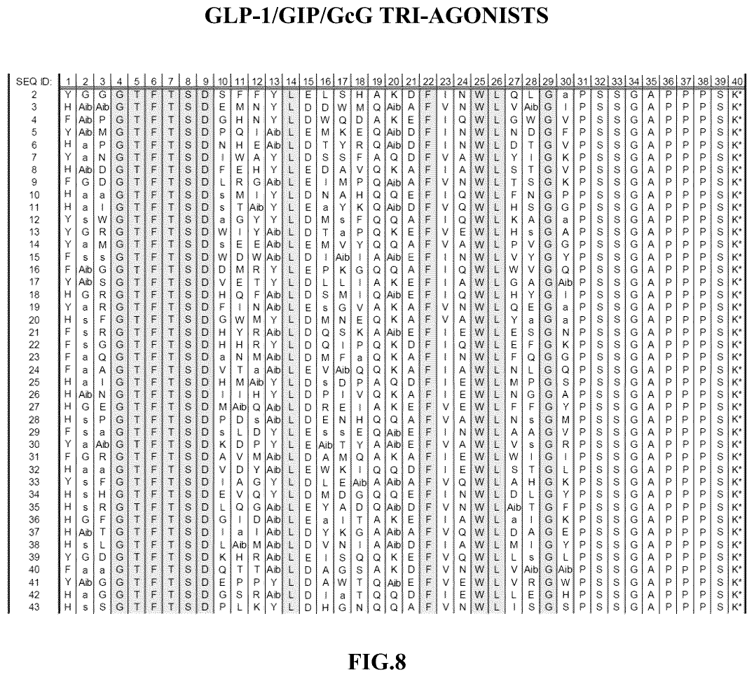

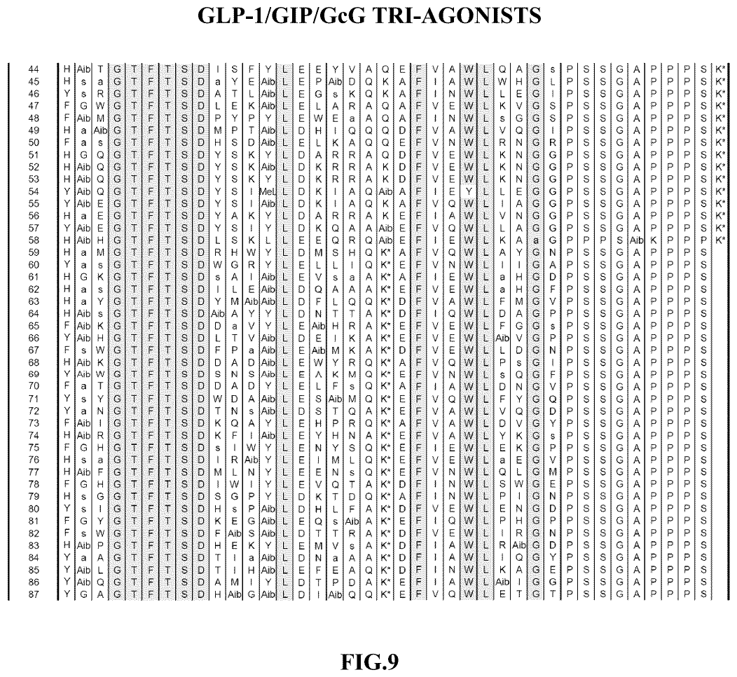

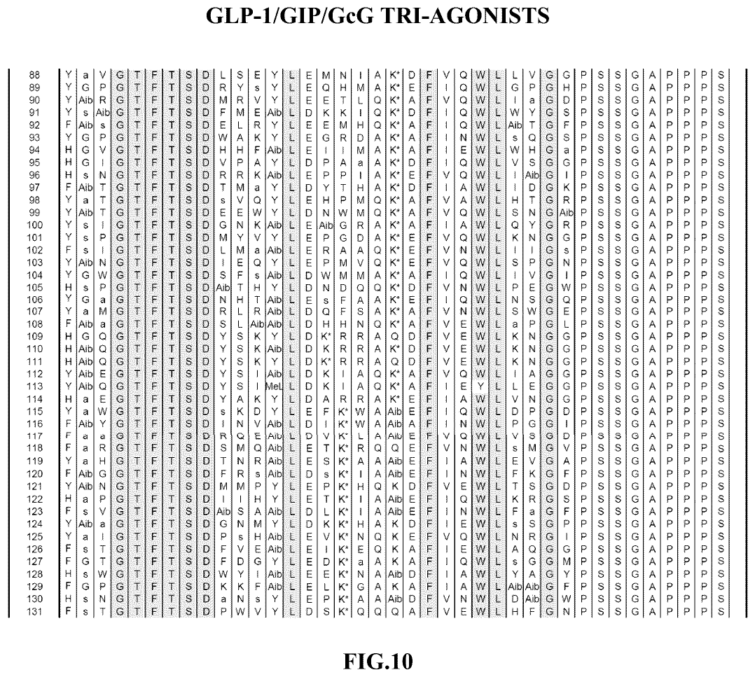

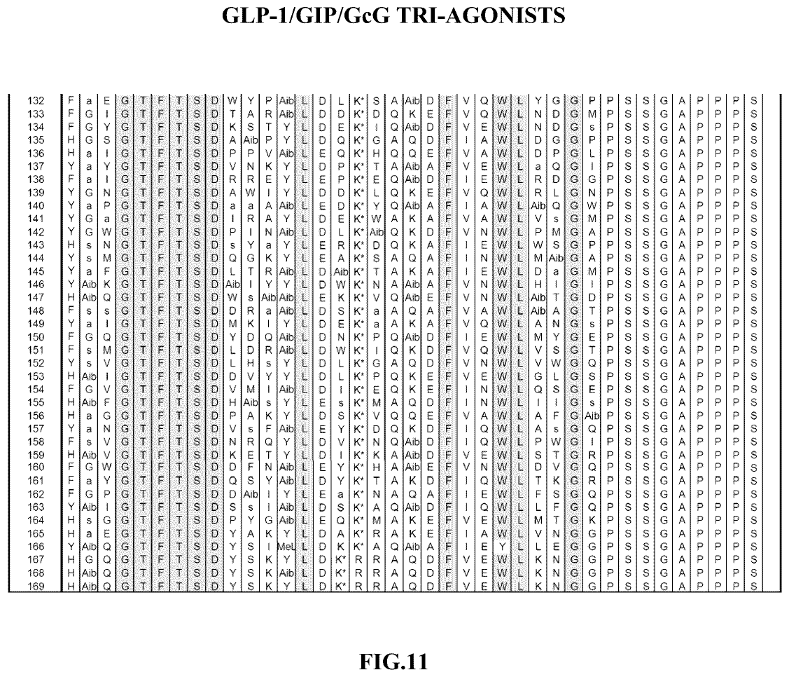

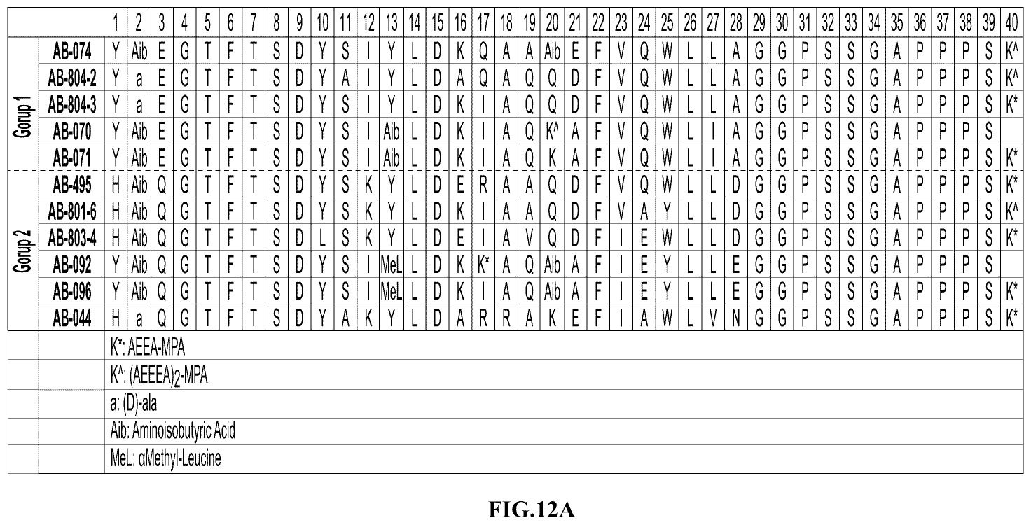

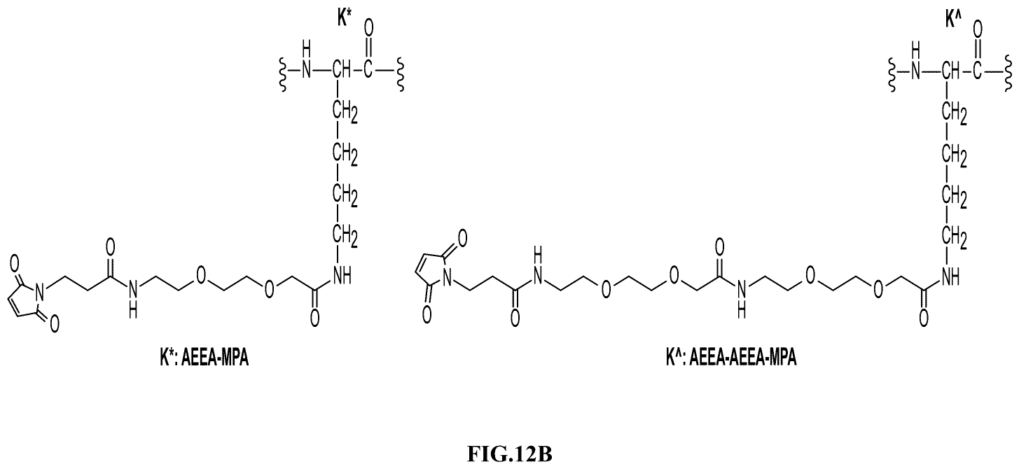

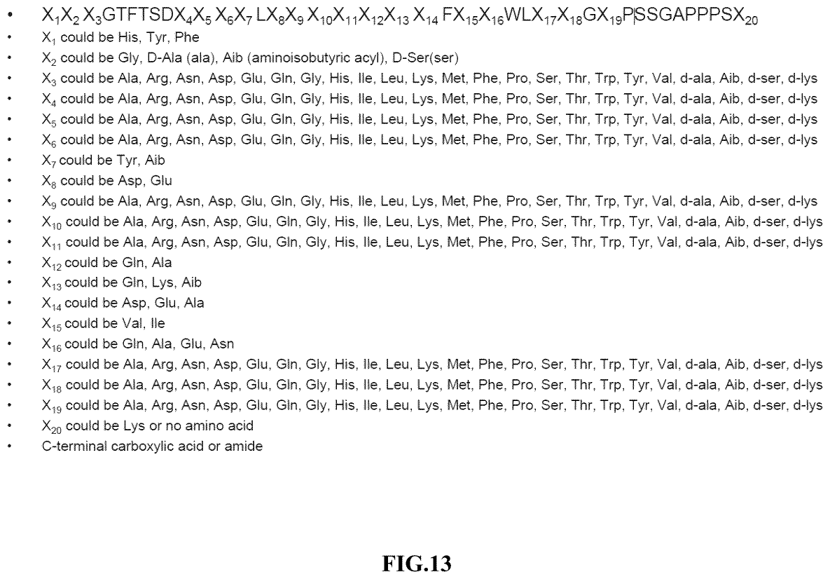

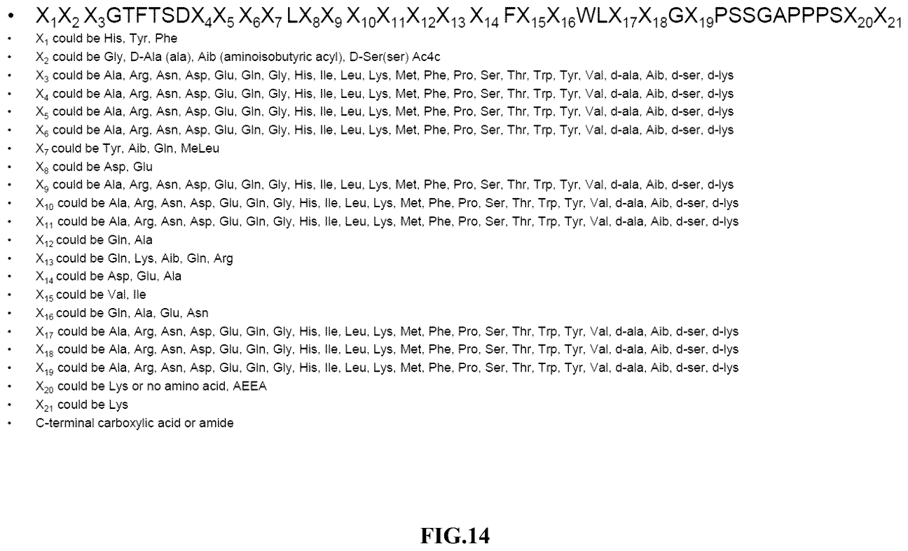

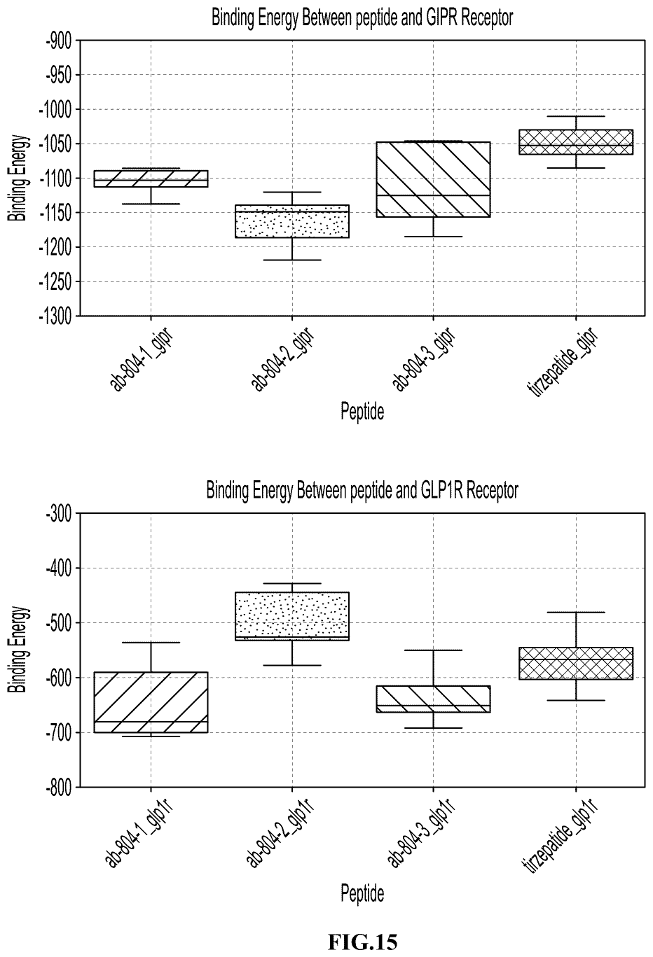

Exemplary agonist peptides, each of which may be further bound to a linker with a coupling group, particularly include GLP-1 agonists, dual agonists, and tri-agonists as shown in FIGS. 4-11, and as listed below. Here, and unless indicated otherwise, K* denotes a modified lysine with an AEEA-MPA, AEEA-AEEA-MPA, AEEA-OA-MPA, or AEEA-OA-AEEA-MPA linker, where the lysine may or may not be preceded by an AEEA group. However, it should be appreciated that various alternative linkers with a coupling group may beused, including MPA, (bromo) MPA, AEEA, AEEA-(bromo) MPA, (AEEA) 2-(bromo) MPA, AEEEA-OA-(bromo) MPA, AEEA-OA-AEEA-(bromo) MPA, (AEEEA) 2-OA-MPA, and (AEEEA) 2-OA-(bromo) MPA. FIG. 12 depicts still further exemplary agonists and linker groups. FIG. 13 and FIG. 14 depict a generic sequence for tri-agonists where X₂₀ or X₂₁, when lysine, can be a modified lysine with an MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA linker, where the so modified lysine may or may not be preceded by an AEEA group.

While contemplated agonist peptides can be tested for their binding and activation parameters using in vitro cell-based experiments, SPR experiments, cell-based experiments, or in vivo tests well known in the art, it should be appreciated that stability and binding of the agonist peptides to the respective receptors may also be investigated in silico measuring complex binding energy and peptide self-energies. For example, the inventors have discovered a preferred sequence (AB-804-3, SEQ ID NO:467, optionally without linker/coupling group) with high binding energy in terms of both complex binding energy and peptide self-energies, which when covalently bound in a retro-Michael-resistant manner, can take advantage of the hydrophobic tryptophan cage with a hydrophilic linker for covalently linking to the free-thiol of Cys₃₄ of recombinant albumin, which may pr may not be conformationally modified. Studies have shown that the peptide domains of AB-804-3 including S1, S2, S3, S4, tryptophan cage, N and C terminus all interact with the extracellular domain and transmembrane domains of their respective receptors (GLP-1 and GIP), and exemplary data show AB-804-3 having highest binding energy.

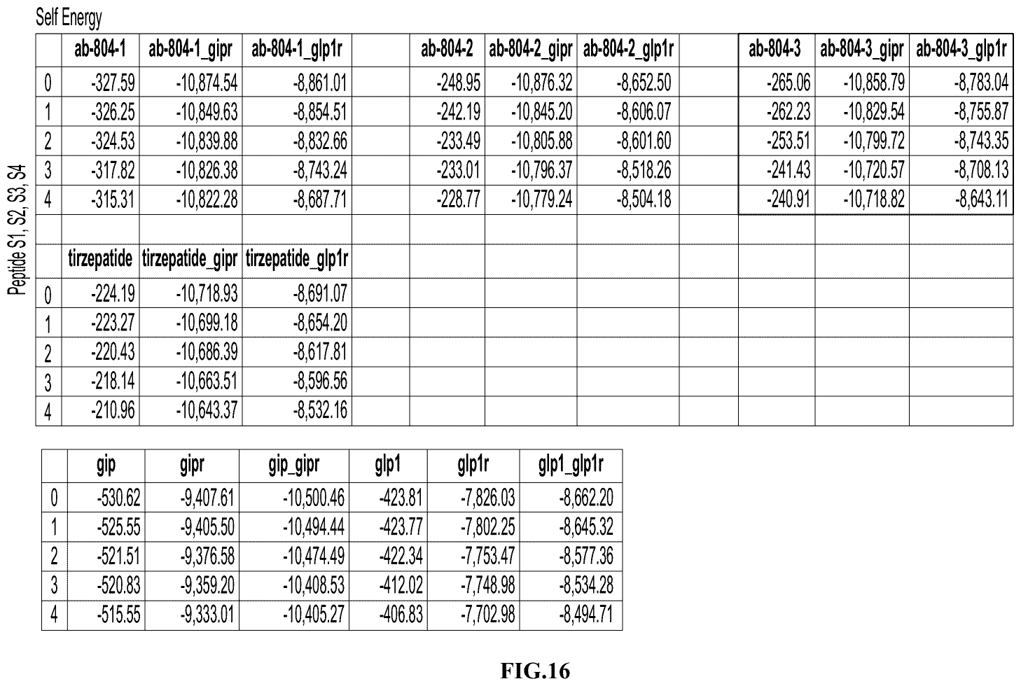

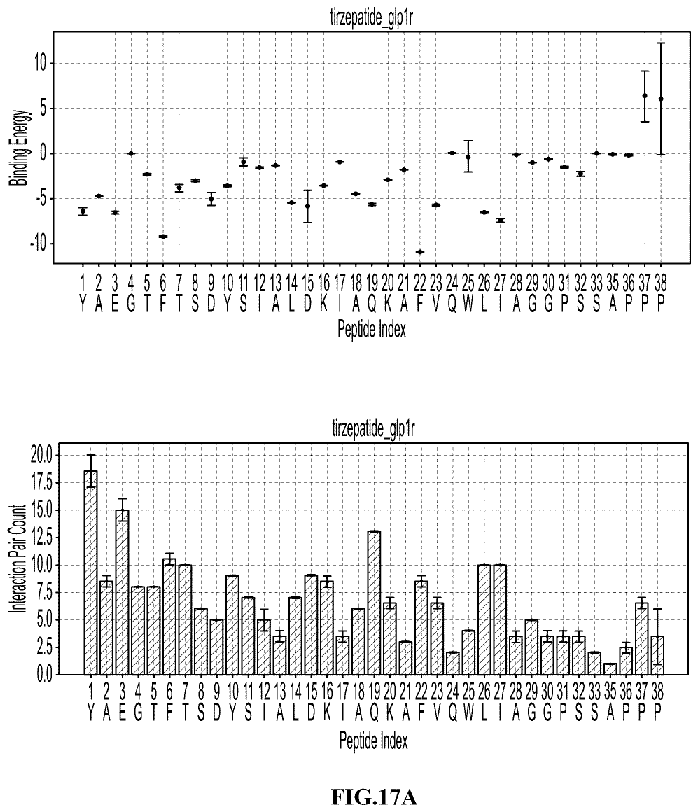

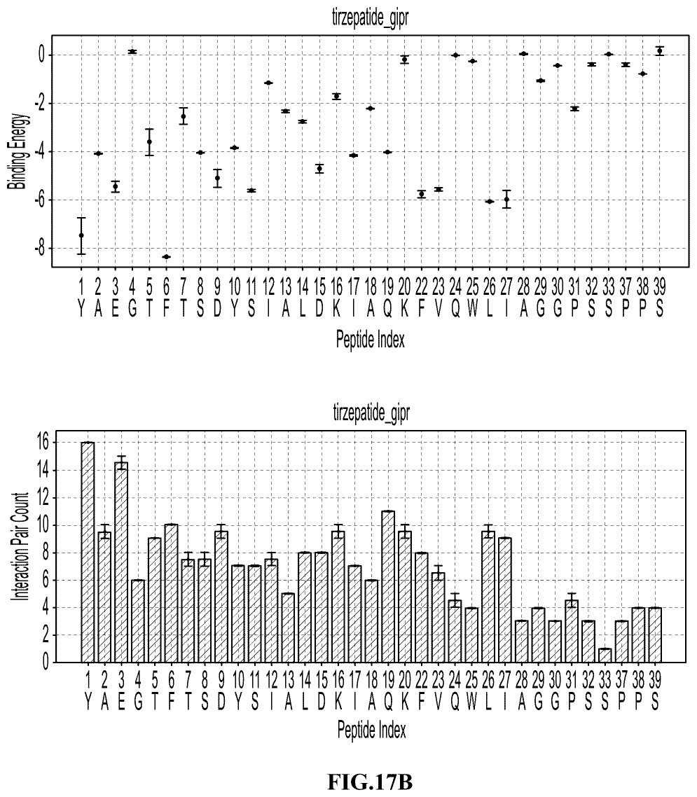

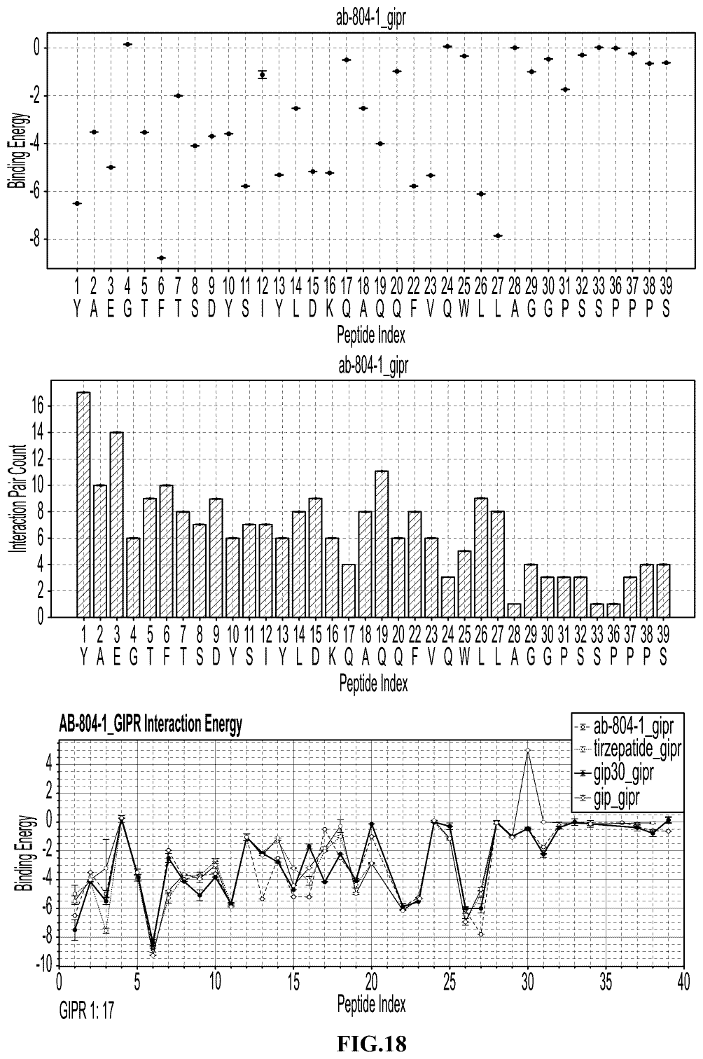

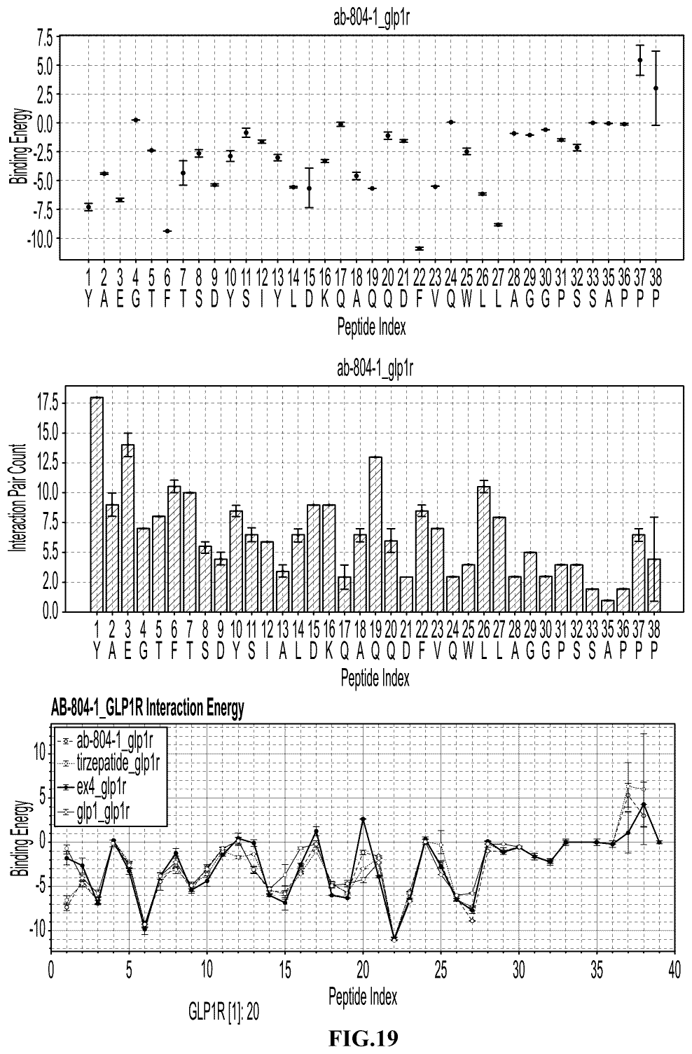

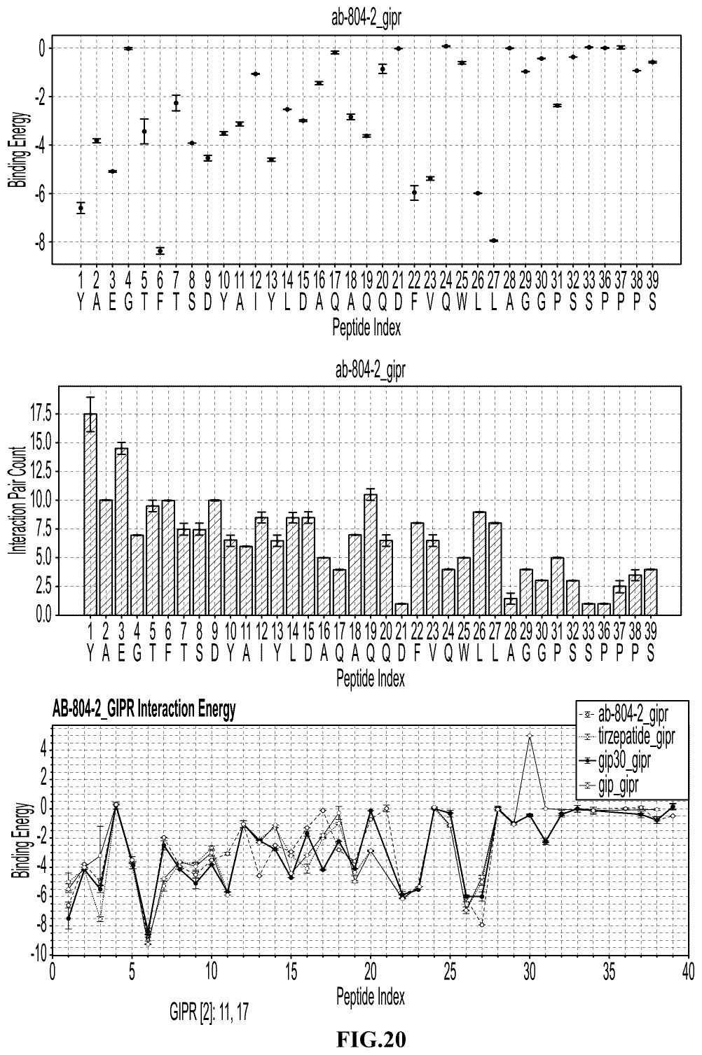

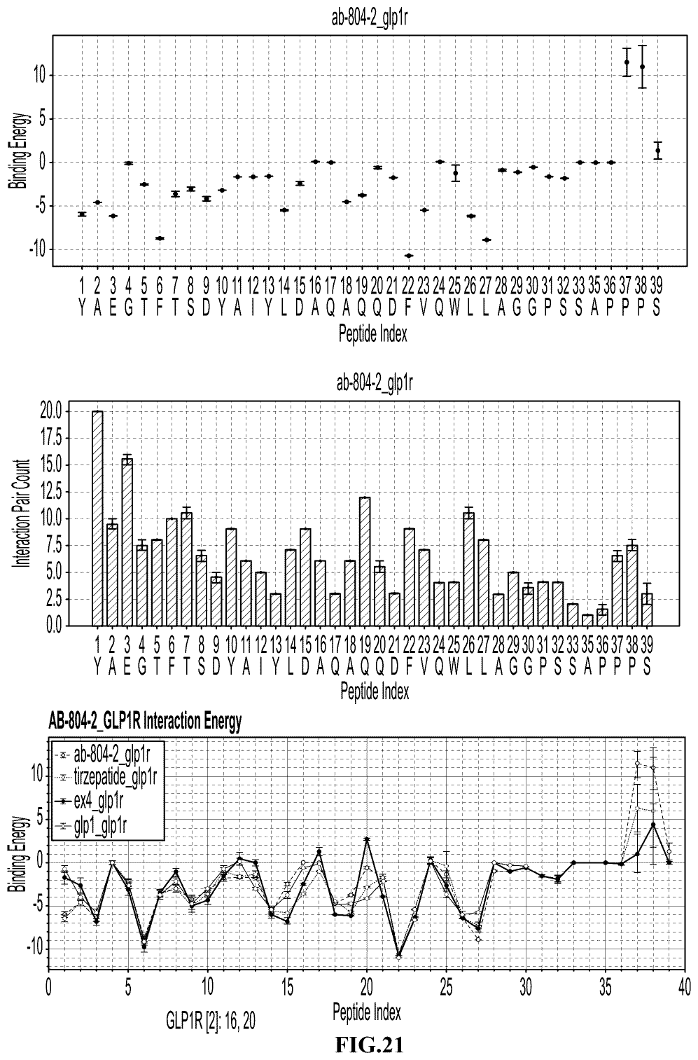

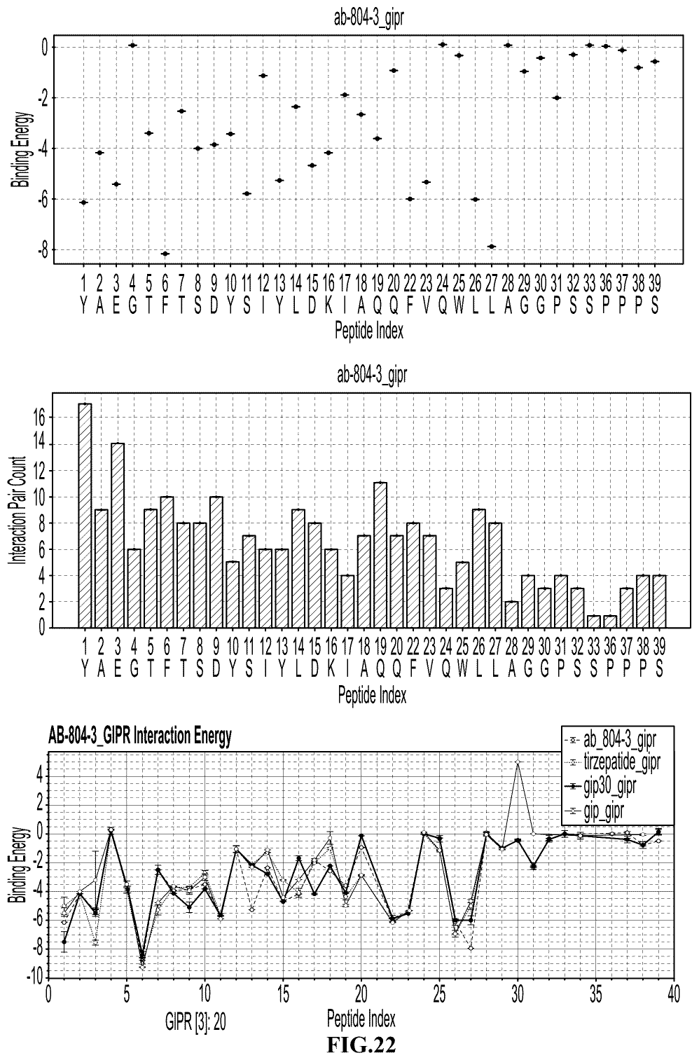

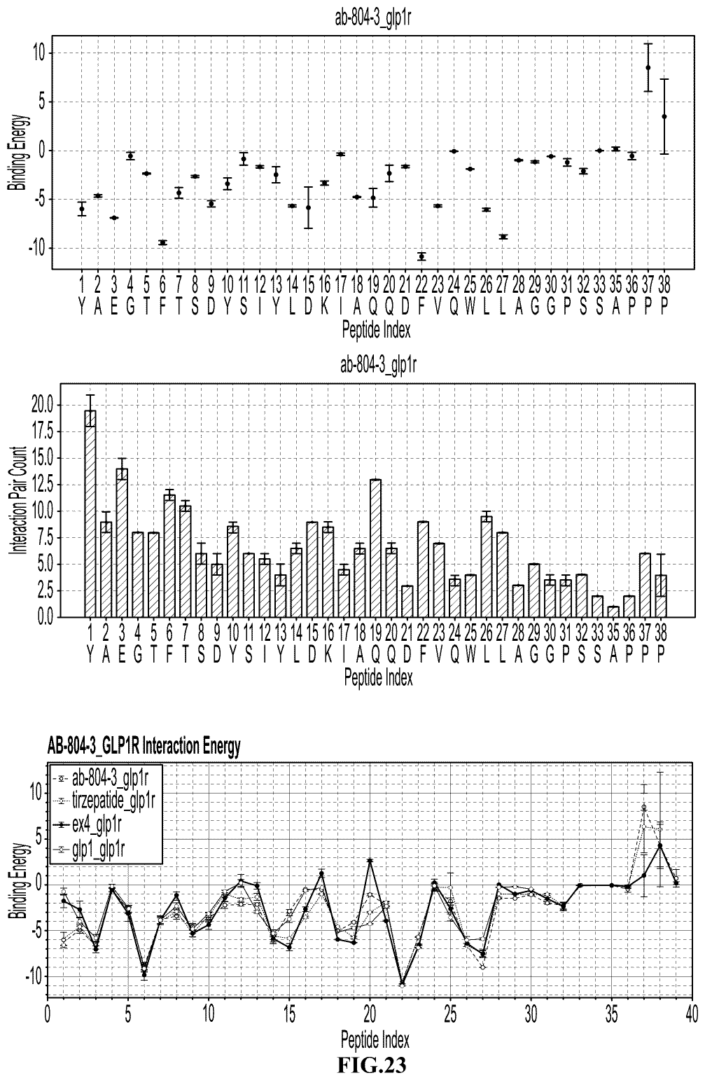

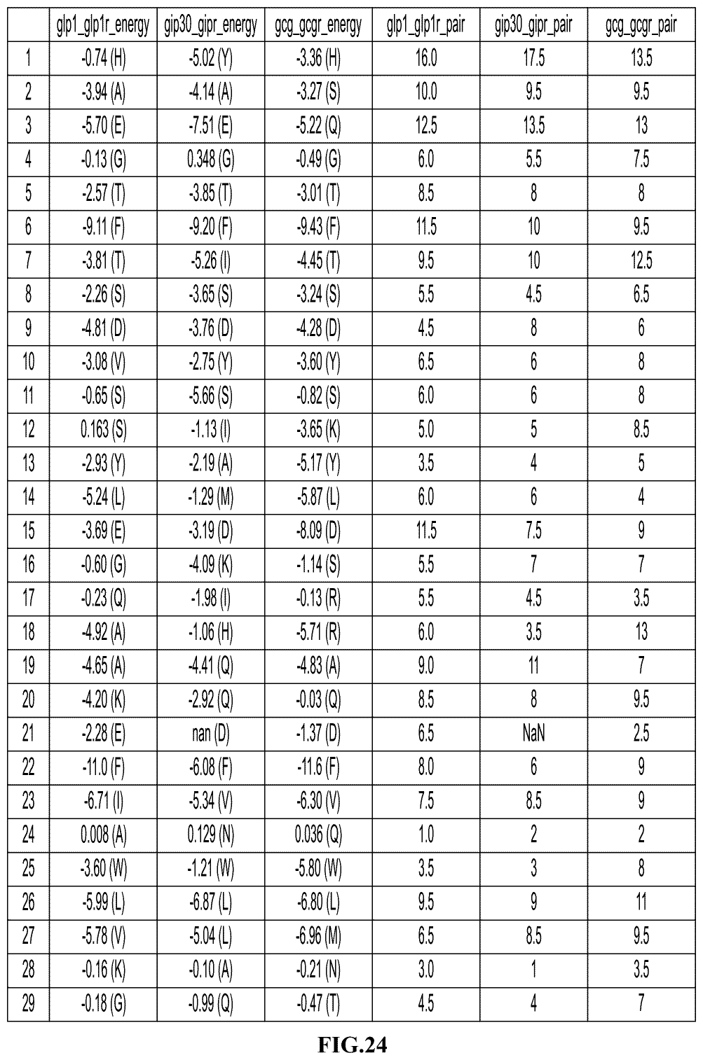

In particular, FIG. 15 depicts binding energy of the tested dual-agonist peptides with the GIP receptor and GLP-1 receptor, and FIG. 16 depicts calculated self-energies for the dual agonists using tirzepatide as control. Binding interactions and interaction pair counts are shown for tirzepatide and GLP-1 and GIP receptors in FIG. 17, for AB-804-1 and GIP receptors in FIG. 18 and for AB-804-1 and GLP-1 receptors FIG. 19. FIG. 20 depicts binding interactions and interaction pair counts for AB-804-2 and GIP receptors, and FIG. 21 depicts binding interactions and interaction pair counts for AB-804-2 and GLP-1 receptors. Similarly, FIG. 22 depicts binding interactions and interaction pair counts for AB-804-3 and GIP receptors, and FIG. 23 depicts binding interactions and interaction pair counts for AB-804-3 and GLP-1 receptors. FIG. 24 provides a comparative table for pair interaction energy for native GLP-1, GIP, and GcG complexes.

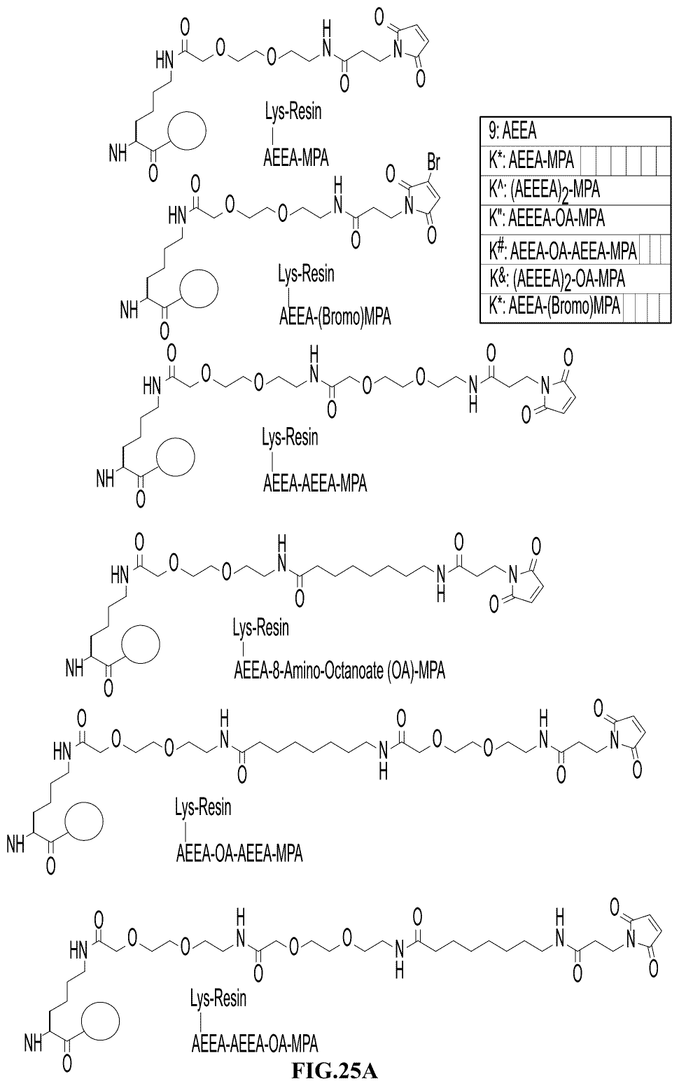

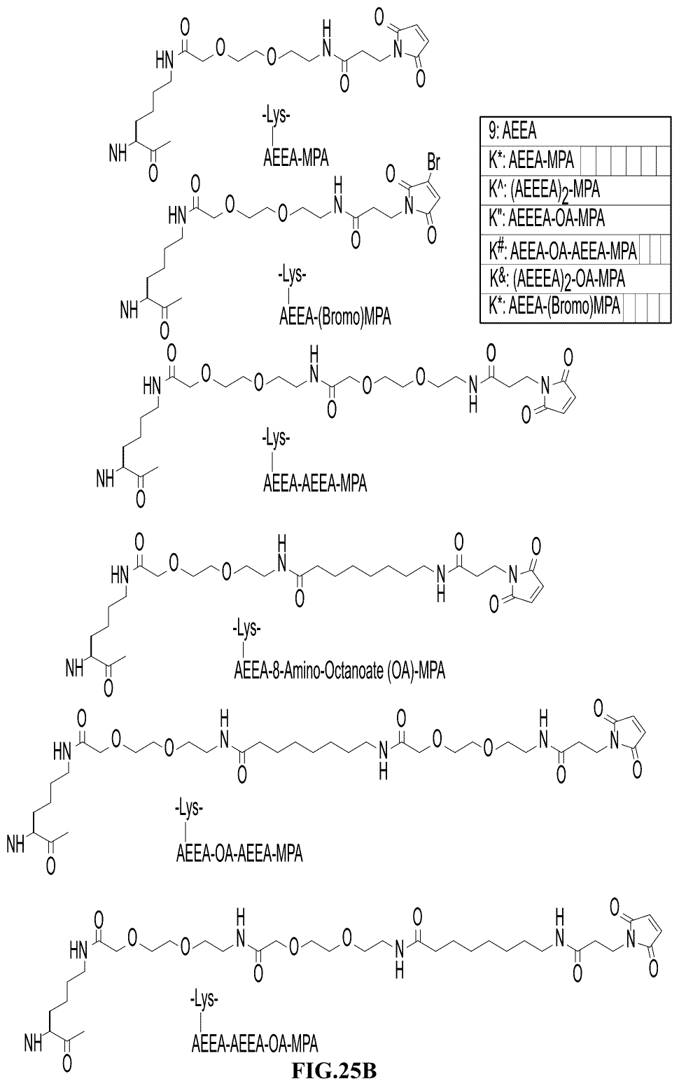

With respect to contemplated linkers that can be used to couple the peptide agonists to the Cys₃₄ thiol group of albumin, it is generally contemplated that preferred linkers will provide sufficient flexibility and steric distance such that the bound agonist peptide will be able to bind (typically in a two-step process) to the GPCR such that the GPCR will be activated. Therefore, in at least some embodiments, the reactive group (e.g., maleimide group) that couples the linker to the Cys₃₄ thiol group of albumin and the group (e.g., amino group) that couples the linker to the agonist peptide will have a linear distance equivalent to at least 6 carbon-carbon bonds, or at least 8 carbon-carbon bonds, or at least 10 carbon-carbon bonds, or at least 12 carbon-carbon bonds, or at least 14 carbon-carbon bonds, or at least 18 carbon-carbon bonds, or at least 24 carbon-carbon bonds. As will also be readily appreciated, the linker may further include one or more non-carbon atoms to so form an amide bond, an ether bond, etc. Thus, in at least some embodiments preferred linkers will be hydrophilic linkers. FIG. 25 depicts a selection of exemplary linkers suitable for use herein in which AEEA denotes (amido (ethoxy) ethoxy acetic acyl), MPA denotes maleimidopropionic acid, OA denotes 8-aminooctanoate, and Lys denotes lysine.

In still further embodiments it is contemplated that the reactive group that couples the linker to the Cys₃₄ thiol group of albumin is a thiol reactive group such as a maleimide group, a bromo-maleimide group, a succinimide group, etc. However, it is particularly preferred that the reactive group that couples the linker to the Cys₃₄ thiol group will be a group that reacts with a cysteine in a Michael addition reaction. The Michael reaction or Michael 1,4 addition is a reaction between a Michael donor, such as an enolate, thiolate or other nucleophile and a Michael acceptor, usually an α, β-unsaturated carbonyl, such as an MPA to produce a Michael adduct by creating a carbon-carbon or carbon-sulfur bond at the acceptor's β-carbon. In this context, it should be particularly appreciated that upon Michael reaction of a thiol group with a planar maleimide group, absent any stereochemically biasing factors, the reaction product includes a newly generated chiral carbon atom (to which the sulfur atom is covalently bound), which will result in a racemic product. Viewed from a different perspective, a Michael addition reaction product, absent any stereochemically biasing factors, will be an equal mixture of R- and S-configured chiral carbon atoms.

Moreover, it should be appreciated that (as with any other chemical reaction) a Michael addition reaction is a reversible reaction unless other factors will hinder such reaction. Indeed, such retro-Michael addition reaction is well known in the context of antibody-drug conjugates where a drug can readily de-conjugate in a retro-Michael addition reaction where the drug was attached to an exposed thiol group.

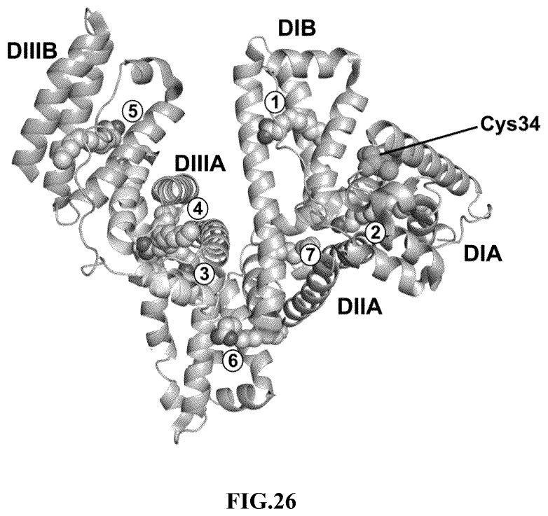

In view of the above, the inventors have unexpectedly discovered that a peptide agonist with a linker having a maleimide or bromo maleimide coupling group can be coupled to Cys₃₄ of albumin in a stereoselective and retro-Michael resistant manner such that the peptide agonist remains covalently bound to the albumin without decoupling. More specifically, the inventors recognized that as the Cys₃₄ is located in a hydrophobic crevice that is 9.5-10 Å deep in which the walls of the crevice are populated by multiple amino acid side chains, the spatial organization surrounding the Cys₃₄ thiol group can have a substantial impact on the stereo-specificity of the newly created chiral center. Surprisingly, the inventors noticed that the spatial organization of the hydrophobic crevice can be modulated by changing the levels of sodium octanoate (or other fatty acids or hydrophobic ligands) present in the hydrophobic fatty acid binding pockets of albumin during the bioconjugation process. In particular, the inventors observed that where a Michael addition was performed when all or most hydrophobic fatty acid binding pockets were occupied by octanoate or other hydrophobic molecule, the reaction product was substantially a racemic mixture (i.e., a 50/50 mix of R- and S-configured chiral carbon in the conjugate), whereas stereospecificity of the reaction increased with decreasing octanoate levels in the binding pockets. Moreover, this unexpected finding was further accentuated where the pH during the conjugation reaction was below pH 7.0. FIG. 26 depicts human albumin with domains DIA through D3B, Cys₃₄ as indicated, and fatty acid binding pockets highlighted by numbers 1-7.

Without wishing to be bound by any theory or hypothesis, the inventors contemplate that fatty acid (or other hydrophobic ligand) binding to albumin changes the 3D conformation in such a manner that the thiol group at Cys₃₄ will be preferentially or be even exclusively available for a Michael addition reaction in only one plane of the maleimide ring and as such preferentially or even exclusively produce only one stereoisomer, resulting in a stereoselective chiral product. Moreover, and particularly at a lower pH (i.e., pH<7.0) the resulting micro-environment inside the “Cys₃₄ hydrophobic crevice” becomes prohibitive to the mechanistic steps required to support a retro-Michael reaction. Hence, unanticipatedly, the stereospecificity of the bioconjugation adduct and its corresponding microenvironment in conformationally modified albumin guard against retro-Michael bond Cys₃₄ sulfur-carbon (MPA) cleavage.

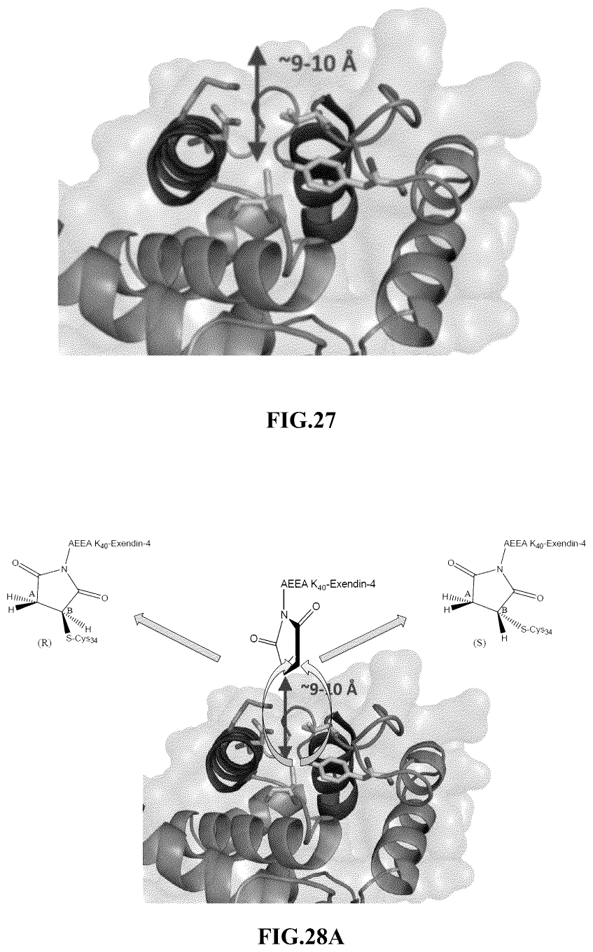

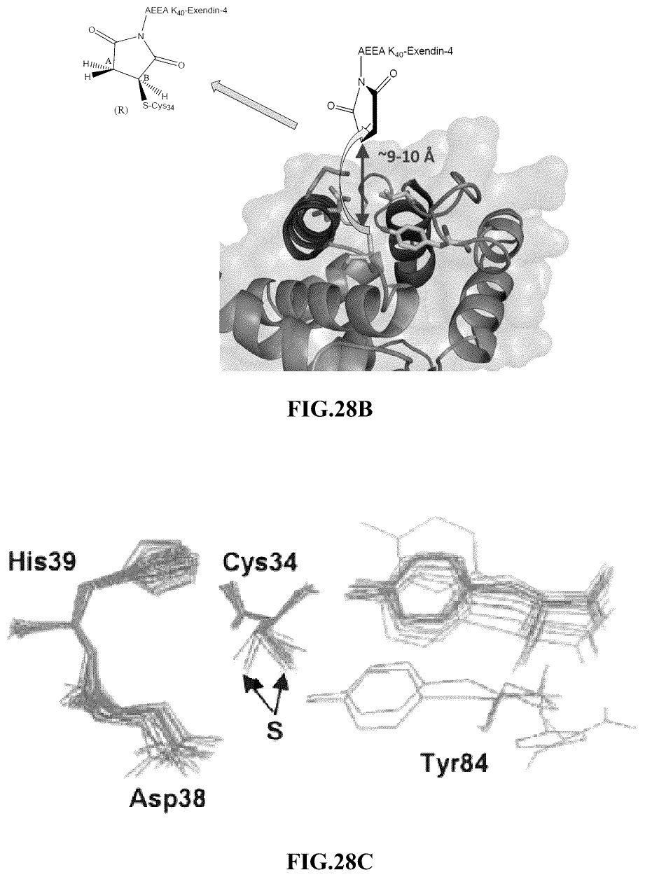

FIG. 27 depicts a side view of the Cys₃₄ hydrophobic crevice with the thiol pointing upwards near the bottom of the crevice and the Asp38 side chain pointing towards the center. In this unmodified albumin conformation, the hydrophobic pockets of albumin are occupied by octanoate, and the thiol group can participate in a reaction on both sides of a planar maleimide group. FIG. 28A depicts a schematic detail view of FIG. 27 where the Asp38 carboxylate portion and thiol group of Cys₃₄ form hydrogen bonds with a water molecule. As is schematically shown by the two arrows in FIG. 28A, the Michael donor (sulfur atom of Cys₃₄) has indiscriminate access to the Michael acceptor group (planar maleimide ring) with regard to the plane of the acceptor. Consequently, as the reaction product creates a chiral carbon center, and as there is no discrimination with regard to the plane of nucleophilic attack, the reaction product is a racemic product (i.e., substantially equal proportion of R- and S-configuration). Upon removal of one or more hydrophobic ligands from the binding pockets in albumin, and particularly at a pH<7.0 (yielding a at Asp38 protonated carboxylate group) the inventors discovered that the albumin is conformationally modified and that the Cys₃₄ hydrophobic crevice is changed in such a manner that not only stereochemically disfavors a retro-Michael deconjugation but also results in a stereopreferred or even stereoselective reaction product as is exemplarily depicted by the single arrow in FIG. 28B. the change in conformation of albumin is exemplarily depicted in FIG. 28C that illustrates a structural overlay of conformationally unmodified (dark gray) and conformationally modified albumin (light gray) prepared by aligning the backbone atom of residues Cys₃₄ and His39 in each of the structures using Swiss pdb viewer (v3.7) (FEBS Journal 272 (2005) 353-362).

Unexpectedly, despite the relatively small changes in conformation in the hydrophobic pocket, the previously ‘open’ environment surrounding Cys₃₄ in the unmodified albumin changed to a stereochemically constrained environment. In addition, and as can also be seen from FIG. 28C, the sulfur atom of Cys₃₄ is now oriented away from Tyr₈₄. Such changes resulted in the generation of a chiral environment that now favored or even restricted the nucleophilic attack of the sulfur atom to a single plane of the planar Michael acceptor resulting in a stereoisomer that was retro-Michael resistant due to steric constraints in the hydrophobic pocket.

In contrast, retro-Michael reactions in other compositions (e.g., various antibody-drug conjugates) were heretofore only avoided by hydrolysis of the succinimide group formed after Michael addition. Such hydrolysis, however, occurs at a pH>9 and temperatures ranging from 37-50° C. over a period of 24-60 hours. Clearly, under such conditions both the protein and the pharmacophore, especially if peptide-based, will undergo deamidation on amino acid amide side chains, which would lead to loss of function in the GPCR agonist compounds presented herein.

As such, it should be recognized that the inventors were now able to produce an albumin carrier that covalently bound and retained a GPCR agonist such that upon administration of the compound substantially no GPCR agonist was released, leading to highly effective compounds without attendant release of agonist, thereby eliminating adverse effects associated with free or deconjugated agonists otherwise encountered. Moreover, due to the nature of the linker attachment to the albumin and the agonist as discussed in more detail below, it should also be appreciated that the agonist is sterically free to engage with the GPCR such that the agonist can bind with high affinity and such that the agonist can activate the GPCR in a two-step manner.

With respect to the removal of octanoate or other hydrophobic compounds including fatty acids from albumin it is contemplated that all methods that are able to reduce the octanoate or other hydrophobic compounds without denaturing the albumin are deemed suitable for use herein, and especially preferred methods include charcoal filtration, dialysis, ion exchange chromatography, etc. (see e.g., Biochim Biophys Acta 1970 Nov. 17;221 (2): 376-8; Nature Communications Materials; (2020) 1:45). As will be readily appreciated, the albumin is preferably human serum albumin, which may be isolated and purified from human serum, or may be recombinantly produced using a bacterial, or more typically, yeast expression system. Thus, albumin contemplated herein may or may not be N or O glycosylated. In this context it should be appreciated that commercially available highly purified natural or recombinant albumin is typically stabilized with exogenously added octanoate or other short or medium-chain fatty acid.

Moreover, it should be appreciated that the albumin can be entirely or only partially defatted such that at least one, or at least two, or at least three, or at least four, or at least five, or at least five, or at least six, or all hydrophobic fatty acid binding pockets of the albumin are free from a hydrophobic ligand to so form the conformationally modified albumin. The suitable degree of defatting will be at least in part determined by the desired degree of a stereoselective reaction product. Thus, and most typically, the albumin will be fully (all hydrophobic fatty acid binding pockets of the albumin are free from a hydrophobic ligand) or almost fully (at least four hydrophobic fatty acid binding pockets of the albumin are free from a hydrophobic ligand) defatted. Once defatted, the so prepared conformationally modified albumin is then reacted with the linker containing the coupling group (typically comprising a maleimide or bromo-maleimide group) following standard Michael addition reaction conditions, preferably at an equimolar ratio, and preferably at a pH that is pH<7.0 (e.g., pH between 6.0 and 6.9, or pH between 5.5 and 6.0, or pH between 5.0 and 5.5, or pH between 4.5 and 5.0, or pH between 4.0 and 4.5). However, excess albumin during the coupling reaction is also deemed suitable, such as a molar excess of 1.1-fold, or 1.2-fold, or 1.3-fold, or 1.4-fold, or 1.5-fold, and even more. Exemplary reaction conditions for coupling the linker to the Cys₃₄ of the conformationally modified albumin are described, for example at Endocrinology (2005) 146 3052-3058, Bioorg. Med. Chem. Lett. (2003) 13 3571-3575, and Bioconjug. Chem. (2005) 16 1000-1008, each of which are incorporated by reference herein.

As such, it should be noted that the inventors discovered that conformationally modified albumin can be readily prepared in solution and used in a variety of biochemical reactions that can take advantage of the conformational modification, and in particular of the modification in the steric environment of the hydrophobic crevice in which Cys₃₄ is located. Most advantageously, such modified environment can lead to stereopreferred or even stereoselective reactions, and with that enantiomeric preferred or even chirally pure reaction products. For example, where the conformationally modified albumin has at least one (or at least two, or at least three or at least four, or at least five, or at least six) hydrophobic pocket not occupied by a hydrophobic ligand such as octanoate, a stereopreferred or even stereoselective Michael addition can be performed using a (typically planar) Michael acceptor reagent. Among other Michael acceptor reagents, maleimide or bromo maleimide-containing reagents are particularly contemplated where the Michael donor is the thiol group of Cys₃₄. Consequently, and as already discussed above, a retro-Michael resistant product (e.g., containing a linker and Class B GPCR agonist peptide as discussed herein) can be readily obtained from a reaction intermediate, particularly where the pH is pH<7.0.

Therefore, and viewed from a different perspective, it should be appreciated that albumin can be modified at Cys₃₄ by reacting the sulfur atom of the Cys₃₄ with a coupling group, wherein the albumin is a conformationally modified albumin in which at least one hydrophobic pocket of the conformationally modified albumin is not occupied by a non-covalently bound lipid. As noted earlier, such reaction is preferably performed at a pH of pH<7.0, and more typically between 4.0 and 6.0. Advantageously, such reactions will lead to retro-Michael resistant reaction products where the coupling group is a planar Michael acceptor such as a maleimide or bromo maleimide group. As such, these reactions may be used to reduce or even entirely avoid retro-Michael reactions in albumin with Michael conjugates, particularly where such conjugates have therapeutic use such as with the agonist peptides with linkers and reactive group as listed in SEQ ID NO:1-471.

In yet another unexpected finding, the inventors discovered that even when octanoate levels are at higher levels during conjugation, a retro-Michael resistant fusion protein can nevertheless be generated via a stereoselective translocation to an unreacted albumin having an unreacted Cys₃₄ group (which may or may not be conformationally modified) in a pharmaceutical formulation post standard purification. Alternatively, the conjugation ratio of peptide intermediate with a linker and MPA as a reactive coupling moiety to the albumin can be adjusted such as to provide an excess amount of unreacted albumin having an unreacted Cys₃₄ group (which may or may not be conformationally modified) when the purification step is an ultrafiltration or diafiltration step, leading to a formulation that also contains an excess amount of unreacted albumin. This unexpected finding was based on the following observations:

Utilizing an albumin-exendin-4 MPA conjugated construct very similar to AB-013 in which albumin was not conformationally modified and in which the construct was in an aqueous solvent at pH 7.4, it was observed that such a construct underwent significant and massive retro-Michael deconjugation as determined by a sandwich ELISA within the first 10 minutes after being injected intravenously in mice. Based on this observation it was concluded that the conjugated peptide was cleaved from the albumin in a retro-Michael reaction and that the only remedy to prevent such reaction was hydrolysis of the succinimide ring in the coupling group by increasing the pH (i.e., pH>9) at elevated temperatures for a lengthy period of time to so stabilize the thiol bond.

To assess the pharmacokinetic profile of both the conformationally unmodified AB-013 conjugate and any free unconjugated peptide with the linker and MPA group that would be released in plasma, the inventors performed a study over 96 hours after subcutaneous or intravenous injection. To that end, an experiment was conducted in rats where the conformationally unmodified AB-013 conjugate was injected intravenously at a dose of 100 nmol/kg and subcutaneously at a dose of 500 nmol/kg. The conformationally unmodified AB-013 conjugate and the anticipated retro-Michael free peptide were quantitated separately via respective ELISA tests but within the same following time points: 2 min, 30 min, 1, 2, 4, 8, 24, 48, 72 and 96 hours for both routes of administration.

From this study, the inventors observed at the first intravenous (I.V.) time point a concentration for conformationally unmodified AB-013 and free unconjugated AB-013 intermediate to be 2528.8 nmol/L and 24.9 nmol/L respectively (leading to a ratio (0.98%). In addition, from the pharmacokinetic profile, the total area under the curve (AUC) I.V. for the conformationally unmodified AB-013 was 37418 (nmol/L*h) whereas the AUC for the free unconjugated material was 53.69 (nmol/L*h) leading to a ratio of 0.14%. Surprisingly, the conformationally unmodified AB-013 and unconjugated “free” peptide were respectively responsible for 99.86% and 0.14% of the presence of the drug in the plasma intravenously. Similarly, for subcutaneously (S.C.) administration, the pharmacokinetic profile leads to a total area under the curve (AUC) for the conformationally unmodified AB-013 of 18944 (nmol/L*h) whereas the AUC for the free unconjugated peptide was 143 (nmol/L*h) leading to a ratio of 0.7%.

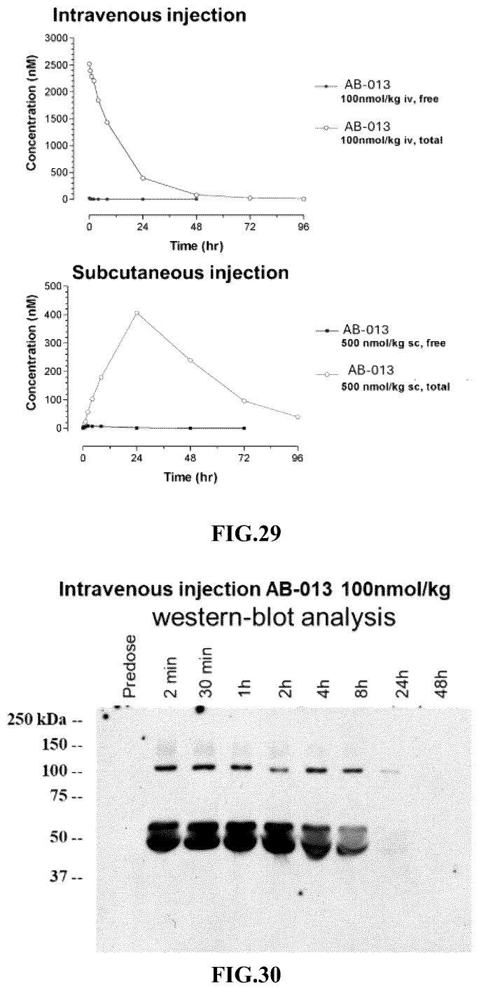

Surprisingly, and contrary to the above observation of retro-Michael decoupling in the range of 20-40% during the first 10 mins post intravenous injection, no such retro-Michael reaction leading to a disconnection between the thiol of Cys₃₄ of albumin and the reactive moiety MPA was observed from this particular experiment. Nor did the inventor observe any pharmacokinetic parallel between the conformationally unmodified AB-013 and the free unconjugated or freshly formed retro-Michael product. Such parallel would have suggested that a retro-Michael reaction played a major role in the degradation of the conformationally unmodified AB-013. FIG. 29 depicts exemplary results for the above experiment.

In yet a further experimental set-up, utilizing a conformationally unmodified albumin-exendin-4 MPA driven covalent construct very similar to conformationally modified AB-013 it was reported that when such conformationally unmodified construct was placed in a solution with a pH>9 and warmed at 37° for 20 hours that a retro-Michael deconjugation occurred and that the deconjugation can be monitored by mass spectrum analysis. Interestingly, the retro-Michael process occurred in only 50% of the MPA driven covalent construct used in that study, leading to the assumption that some hydrolysis of the succinimide ring had occurred.

To further investigate the apparent inconsistencies supplementary electrophoresis experiments were conducted on the plasma samples collected from the rats that were injected s.c. and i.v. as noted above, and exemplary results are depicted in FIG. 30. As can be readily seen in the gel image, when conformationally unmodified AB-013 is tested in an electrophoresis experiment only one band is observed in the area where an albumin-exendin-4 MPA covalent construct would be observed. Completely unexpectedly, all time points taken during the experiment, including the earliest ones, had two bands in the area related to an albumin-exendin-4 MPA covalent construct. The first band observed on electrophoresis was related to conformationally unmodified AB-013 and the second ban immediately above was related to an exendin-4 MPA covalent construct, but covalently attached to rat albumin whereas the conformationally unmodified AB-013 is a covalent attachment to human albumin.

Hence, the inventors surprisingly discovered a direct translocation, in vivo, of a retro-Michael decoupled exendin-4 MPA construct from human albumin to the Cys₃₄ of rat albumin. Interestingly, the intensity of the two bands observed on the electrophoresis gel are of similar intensity (about 50-50), while there were no detectable levels of free (decoupled) exendin-4 MPA construct. This initial observation was further supported when a dynorphin-A-MPA peptide was injected intravenously to rats. Importantly, this dynorphin-A-MPA peptide was not conjugated to human albumin or any other type of albumin. Hence it was injected with the intent of in vivo bioconjugation to rat albumin. Bioconjugation in vivo did indeed occur with rat albumin as monitored via electrophoresis gel (data not shown). Interestingly, for every time point tested only one band was observed on the electrophoresis gel when dynorphin-A-MPA peptide was injected intravenously. In a similar fashion, the dynorphin-A-MPA peptide was also conjugated to human conformationally unmodified albumin in vitro to form a covalent bond between it and the Cys₃₄ of human albumin. The dynorphin a human albumin covalent construct was injected intravenously to rats. Once more, the inventors then observed a direct translocation, in vivo, from an MPA peptide covalently bound to the Cys₃₄ of human albumin to rat albumin.

These results further confirm the inventors' discovery that a Michael addition occurring in a sterically constrained hydrophobic pocket of the albumin leads to the generation of a chiral center, and that conformational modification of the albumin will result in a conformationally modified hydrophobic pocket that favors or even entirely produces a retro-Michael resistant stereoisomer. In addition, retro-Michael reactions can be further disfavored by lower pH, and especially pH of less than 7.0 and lower. Viewed from a different perspective, for a retro-Michael reaction to occur, appropriate conformational architecture within the hydrophobic pocket is required, along with appropriate stereochemical orientation. Indeed, even when experiments are designed to force a retro-Michael reaction under harsh conditions (pH>9, high temperature (e.g., 50° C.), and long time (about 20 hrs)), retro-Michael reaction is observed at a yield of only about 50% of theoretically possible retro-Michael reaction, strongly implicating the importance of the chirality created during the conjugation process and restrictive nature of the sterically constrained hydrophobic pocket.

In view of the above, it should therefore be recognized that albumin can be chemically modified with a coupling group at Cys₃₄ such that a sulfur atom of the Cys₃₄ is covalently bound to a chiral carbon atom of the coupling group, and such that the chiral atom has a favored stereochemical configuration (instead of a racemic carbon atom where the thiol group is not sterically constrained as discussed above). Indeed, it is contemplated (and has been observed) that the favored stereochemical configuration can be favored at a ratio of at least 60:40, or at least 70:30, or at least 80:20, or at least 85:15, or at least 90:10, or at least 95:5, particularly with increasing degree of defatting (i.e., at least one, or at least two, or at least three or at least four, or at least five, or at least six, or all hydrophobic binding pockets are not occupied by a non-covalently bound lipid).

Therefore, it is noted that the inventors discovered that a chemically modified albumin can be produced, where the albumin is modified at Cys₃₄ with a Michael addition conjugate, and where the Michael addition conjugate is resistant to a retro-Michael addition reaction. Indeed, it should be appreciated that it is now possible to prepare compositions in which a coupling group (carrying a (hydrophilic) linker and optionally a peptide coupled to the linker) can be covalently attached to Cys₃₄ of albumin in a stereopreferred (i.e., at least 55%, at least 60%, at least 75%, at least 80%, at least 85%, at least 90%, at least 95% of all chiral carbon atoms in C—S bonds in coupling groups have a configuration that is retro-Michael resistant) or even stereoselective (between 98 and 100% of all chiral carbon atoms in C—S bonds in coupling groups have a configuration that is retro-Michael resistant) manner, thereby rendering the molecular entity attached to the coupling group retro-Michael resistant. While this chemically modified albumin is utilized for the delivery of insulin-tropic agents, it is contemplated that this form of albumin together with the linker described herein could also serve as delivery systems for other drug conjugates including cytotoxic agents, chemotherapeutic agents in the treatment as a form of targeted therapy or antibody drug conjugates (ADCs) in the field of cancer.

Viewed from a different perspective, in an isotonic buffered (pH 7.0) solution containing contemplated retro-Michael resistant conjugates of albumin and agonist peptide, no more than 40%, or no more than 30%, or no more than 20%, or no more than 15%, or no more than 10%, or no more than 8%, or no more than 6%, or no more than 4%, or no more than 2%, of all bound agonist peptide will dissociate from the albumin in a retro-Michael reaction over a period of 24 hours at room temperature. Therefore, it should be appreciated that the majority of the linkers with covalently bound GPCR agonist peptides will be bound to the Cys₃₄ amino acid of albumin in a retro-Michael resistant manner. Thus, pharmaceutical compositions in which a GPCR agonist peptide is covalently bound to a Cys₃₄ amino acid of albumin in a stereopreferred or stereoselective configuration will be substantially free of unbound GLP-1 agonist peptide (e.g., no more than 10%, or no more than 7%, or no more than 5%, or no more than 3%, or no more than 2% of total agonist peptide in the composition are unbound).

As already noted above, such compositions can be prepared via reacting the coupling group and linker (with or without attached peptide or other molecular entity) to the Cys₃₄ of a conformationally modified albumin, by performing the Michael addition in the presence of free albumin in molar excess relative to the coupling group, and/or by adding free albumin to a Michael addition product that was produced by reacting Cys₃₄ of an albumin with a coupling group and linker (with or without attached peptide or other molecular entity such as a linker-bound GPCR agonist peptide). As will be readily appreciated, the ratio of free albumin to albumin with a ligand bound to Cys₃₄ can vary, and suitable ratios will typically be 1:5, or 1:4, or 1:3, or 1:2, or 1:1.5, or 1:1.2, or 1:1, or 1.2:1, or 1.5:1, or 2:1, or 3:1, or 4:1, or 5:1. In each of the cases where additional albumin is used, such albumin can be conformationally modified and/or conformationally unmodified albumin. Moreover, it should be recognized that once the coupling reaction of the linker (typically also containing the agonist peptide) with the albumin is concluded, the GPCR agonist fusion protein can then be purified using standard methods well known in the art. Moreover, and where desired, octanoate or hydrophobic agents may be added to the fusion protein to re-occupy the hydrophobic binding sites, thereby improving stability of the fusion protein.

Returning back to contemplated linkers, it should be noted that the linker that is coupled to the conformationally modified albumin will already be covalently bound to the GPCR agonist peptide, typically via an epsilon amino group of a lysine, preferably located at the C-terminus of the agonist peptide.

While at least conceptually simple, synthesis of the GPCR agonists that also contain a reactive linker is far from trivial, particularly where an automated Merrifield-type synthesis is desired. Synthesis is even further complicated where the agonist is exendin-4 or an exendin-4 derivative that contains a tryptophan cage at the C-terminus, which presents substantial steric hindrance for any chemistry proximal to the C-terminus. In addition, and particularly where the agonist peptide is coupled to a linker via a second reactive group, differential protection and deprotection is required, which may yet again be difficult to achieve in the proximity to the synthetic resin. Viewed from a different perspective, while linear GLP-1 analogs have been prepared on a solid phase as described in U.S. Pat. No. 6,514,500, protocols to perform orthogonal site-specific chemistries on the very first amino acid that is anchored on a solid support have not been established, nor are there known synthetic protocols for performing orthogonal site-specific chemistries on the very first amino acid anchored on a solid support when the structure of the peptide is highly organized at the C terminus end.

In view of the above difficulties, the inventors unexpectedly discovered that successful protocols to perform orthogonal site-specific chemistries on the first AA anchored on a solid support required a preferred distance between the solid support (here: polystyrene beads) and the anchoring site of the first amino acid. In one particularly preferred embodiment, a tricyclic amide resin (Ramage resin) provided the necessary and preferred distance to successfully perform orthogonal protection/deprotection. Advantageously, the inventors also recognized that such tricyclic amide resin was also suited for large scale and cGMP scale manufacturing.

In the following example, the small-scale peptide synthesis of AB-013 intermediate was performed on the modified tricyclic amide resin (Ramage resin) using an automated solid-phase procedure on a Symphony® peptide synthesizer with manual intervention to perform orthogonal chemistries of incorporating a linker like AEEA (Amido (Ethoxy) Ethoxy] Acetic acyl) and a reactive moiety such as maleimidopropionic acyl. As should be readily appreciated, the same synthetic strategy can also be implemented for the other GPCR agonist peptides presented herein.

As will be readily appreciated, the suitable choice of the resin substitution level will be a function of the desired synthetic efficiency, the length of the peptide intermediate, and the complexity of the structural conformation in combination with the location along the peptide chain of such complexity. That said, preferred agonist peptides will have a length between 30-45 amino acids with significant structural conformation complexity (here: Trp cage) at the C-terminal end of the peptide. Furthermore, in most (but not all of the contemplated) embodiments, orthogonal chemistries are conducted on the first amino acid that is anchored on the resin. Hence an appropriate distance between the polystyrene and the anchoring site of the first amino acid is preferred as noted above. Advantageously, the modified Ramage resin structure and substitution level addresses synthetic efficiency, length of the peptide intermediate, complexity/location of the structural conformation and orthogonal chemistries on the first amino acid anchored the resin. Preferably, but not necessarily, the substitution level is approximately 0.4-0.6 mmol/gram Ramage resin.

With regard to scale-up, it is noted that the peptide intermediate batch size will be dependent on the scale of the synthesis and the substitution of the Ramage resin. This in turn provides the necessary information regarding the amount of Ramage resin for a particular batch. For example, a 100-mmole synthesis scale using a modified Ramage resin with a substitution level of 0.5 mmol/g, will require an amount of starting Ramage resin of ˜200 g and would have a theoretical yield of ˜8.5% for a 40-mer peptide. Thus, a batch size of 100 mmol would be an appropriate large scale (e.g., cGMP) batch size for process validation, while for a research scale a batch size of 100 umol/g of modified Ramage resin would be appropriate.

The amino acid N-terminus is protected by groups that are termed “temporary” protecting groups, because they are relatively easily removed to allow peptide bond formation. Two common N-terminal protecting groups are tert-butoxycarbonyl (Boc) and 9-fluorenylmethoxycarbonyl (Fmoc), and each group has distinct characteristics that determine their use. Boc requires a moderately strong acid such as trifluoracetic acid (TFA) to be removed from the newly added amino acid, while Fmoc is a base-labile protecting group that is removed with a mild base such as piperidine. Because of the mild deprotection conditions, Fmoc chemistry is more commonly used in commercial settings because of the higher quality and greater yield, while Boc is preferred for complex peptide synthesis or when non-natural peptides or analogs that are base sensitive are required. Hence, the protecting groups contemplated herein fall in two categories: base-labile and acid-labile. The base-labile protecting group is typically used to block the α-amino group during the coupling reaction and is then removed in the deblocking step to allow the introduction of the next amino acid in the sequence. For example, 9-Fluorenylmethoxy carbonyl (Fmoc) can be used as the base-labile, α-amino protecting group. In another example, the acid-labile protecting groups (e.g., triphenylmethyl (Trt)/t-butyl (tBu)/t-butyl ester (OtBu)/t-butyloxycarbonyl (Boc)/2,2,5, 7,8, pentamethylchroman-6-sulfonyl (Pmc)/methyltrityl (Mt)) can be used to protect the side-chain reactive functional groups of the amino acids during synthesis, and must be resistant to the deblocking mixture (e.g., 20% piperidine in dimethylformamide (DMF), v/v). Upon completion of the peptide synthesis, these protecting groups are removed by a strong acid (e.g., 85% aqueous trifluoroacetic acid (TFA) with scavengers).

One particularly preferred method of preparing the peptide agonists presented herein involves solid phase peptide synthesis in which the amino acid N-terminus is protected by an acid or base sensitive group while being readily removable without destruction of the growing peptide chain or racemization of any of the chiral centers contained therein. Examples of N-protecting groups and carboxy-protecting groups are disclosed in Greene, “Protective Groups in Organic Synthesis,” (John Wiley & Sons, New York pp. 152-186 (1981)), which is hereby incorporated by reference. Examples of N-protecting groups comprise, without limitation, lower alkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, -chlorobutyryl, benzoyl,4-chlorobenzoyl, 4-bromobenzoyl, 4-nitrobenzoyl and the like; sulfonyl groups such as benzenesulfonyl, p-toluene-sulfonyl, o-nitrophenylsulfonyl, 2,2,5,7,8-pentamethylchroman-6-sulfonyl (pmc), and the like; carbamate forming groups such as t-amyloxycarbonyl, benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4 dimethoxybenzyloxycarbonyI, 3,5-dimethoxybenzyloxycarbony 1,2,4-dimethoxy-benzyloxycarbonyl, 4-ethoxybenzyloxycarbonyl, 2-nitro-4,5-dimethoxybenzyloxycarbony 1,3,4,5-trimethoxybenzyloxycarbony 1,1-(p-biphenylyl)-1-methylethoxycarbonyl, a, a-dimethyl-3,5-dimethoxybenzyloxycarbonyl, benzhydryloxycarbonyl, t-butyloxycarbonyl (boc), diisopropylmethoxycarbonyl, isopropyloxycarbonyl, ethoxycarbonyl, methoxycarbonyl, allyloxycarbonyl, 2,2,2, -trichloroethoxycarbonyl, phenoxycarbonyl, 4-nitrophenoxycarbonyl, fluorenyl-9-methoxycarbonyl, isobomyloxycarbonyl, cyclopentyloxycarbonyl, adamantyloxycarbonyl, cyclohexyloxycarbonyl, phenylthiocarbonyl and the like; arylalkyl groups such as benzyl, biphenylisopropyloxycarbonyl, triphenylmethyl, benzyloxymethyl, 9-fluorenylmethyloxycarbonyl (Fmoc) and the like and silyl groups such as trimethylsilyl and the like. Preferred a-N-protecting group are o-nitrophenylsulfenyI; 9-fluorenylmethyloxycarbonyI; t-butyloxycarbonyI (boc), isobomyloxycarbonyl; 3,5-dimethoxybenzyloxycarbonyl; t-amyloxycarbonyl; 2-cyano-t-butyloxycarbonyl, and the like, 9-fluorenyl-methyloxycarbonyl (Fmoc) being more preferred, while preferred side chain N-protecting groups comprise 2,2,5,7,8-pentamethylchroman-6-sulfonyl (pmc), nitro, p-toluenesulfonyl, 4-methoxybenzene-sulfonyl, Cbz, Boe, and adamantyloxycarbonyl for side chain amino groups like lysine and arginine; benzyl, o-bromobenzyloxycarbonyl, 2,6-dichlorobenzyl, isopropyl, t-butyl (t-Bu), cyclohexyl, cyclopenyl and acetyl (Ac) for tyrosine; t-butyl, benzyl and tetrahydropyranyl for serine; trityl, benzyl, Cbz, p-toluenesulfonyl and 2,4-dinitrophenyl for histidine; formyl or Boc for tryptophan; benzyl and t-butyl for aspartic acid and glutamic acid; and triphenylmethyl (trityl) for cysteine.

A carboxy-protecting group conventionally refers to a carboxylic acid protecting ester or amide group. Such carboxy protecting groups are well known to those skilled in the art, having been extensively used in the protection of carboxyl groups in the penicillin and cephalosporin fields as described, for example, in U.S. Pat. Nos. 3,840,556 and 3,719,667, both of which are hereby incorporated herein by reference. Representative carboxy protecting groups include, without limitation, C1-C8 lower alkyl; arylalkyl such as phenethyl or benzyl and substituted derivatives thereof such as alkoxybenzyl or nitrobenzyl groups; arylalkenyl such as phenylethenyl; aryl and substituted derivatives thereof such as 5-indanyl; dialkylaminoalkyl such as dimethylaminoethyl; alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyI)-1-ethyI, 1-methy 1-1-(propionyloxy)-1-ethyl, pivaloyloxymethyl, propionyloxymethyl; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl; aroyloxyalkyl such as benzoyloxymethyl, benzoyloxyethyl; arylalkylcarbonyloxyalkyl such as benzylcarbonyloxymethyl, 2-benzylcarbonyloxyethyl; alkoxycarbonylalkyl or cycloalkyloxycarbonylalkyl such as methoxycarbonylmethyl, cyclohexyloxycarbony ImethyI, 1-methoxycarbonyl-1-ethyl; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, 1-cyclohexyloxycarbonyloxy-1-ethyl; aryloxycarbonyloxyalkyl such as 2-(phenoxycarbonyloxy)ethyl, 2-(5-indanyloxycarbonyloxy)-ethyl; alkoxyalkylcarbonyloxyalkyl such as 2-(1-methoxy-2-methylpropan-2-oyloxy)-ethyl; arylalkyloxycarbonyloxyalkyI such as 2-(benzyloxycarbonyloxy)ethyl; arylalkenyloxycarbonyloxyalkyl such as 2-(3-phenylpropen-2-yloxycarbonyloxy)ethyl; as alkoxycarbonylaminoalkyl such t-butyloxycarbonylaminomethyl; alkylaminocarbonylaminoalkyl such as methylaminocarbonylaminomethyl; alkanoylaminoalkyl such as acetylaminomethyl; heterocycliccarbonyloxyalkyl such as 4-methylpiperazinylcarbonyloxymethyl; dialkylaminocarbonylalkyl such as dimethylaminocarbonylmethyl, diethylaminocarbonylmethyl; (5-(loweralkyl)-2-oxo-1,3-dioxolen4-yl)alkyl such as (5-t-butyl-2-oxo-1,3-dioxolen-4-yl)methyl; and (5-phenyl-2-oxo-1,3-dioxolen-4-yl)alkyl such as (5-phenyl-2-oxo-1,3-dioxolen-4-yl)methyl. Representative amide carboxy protecting groups comprise, without limitation, aminocarbonyl and lower alkylaminocarbonyl groups. Of the above carboxy-protecting groups, loweralkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester are preferred. Preferred amide carboxy protecting groups are lower alkylaminocarbonyl groups.

The-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluronium-hexafluorophosphate (HBTU), with or without 4-dimethylaminopyridine (DMAP), 1-hydroxybenzotriazole (HOBT), benzotriazol-1-yloxy-tris (dimethylamino)phosphonium-hexafluorophosphate (BOP) or bis (2-oxo-3-oxazolidinyl)phosphine chloride (BOPCI), or Oxyma Ethylcyanohydroxyiminoacetate, or HATU (1-[bis (dimethylamino)methylene]-1H-1,2,3-triazolo[4,5-b]pyridinium 3-oxide hexaflurophosphate, or TBTU 2-(1H-benzotriazolel-yl)-1,1,3,3-tetramethylaminium tetrafluoroborate, or TNTU 2-(5-Norbornene-2,3-dicarboximido)-1,1,3,3-tetramethyluronium tetrafluoroborates, or PyOxim 1-Cyano-2-ethoxy-2-oxoethylideneaminooxy-tris-pyrrolidino-phosphonium hexafluorophosphate, or PyClock 6-chloro-benzotriazolelyloxy-tris-pyrrolidinophosphonium hexafluorophosphate diethylamine (DEA).

Coupling is performed from about 1 to about 24 hours at a temperature of between 10° C. and 50° C. in a solvent such as dichloromethane, DMF and NMP and/or a solvent mixture compatible with coupling of amino acids and/or acylation.

During the activation/coupling steps, the deprotected α-amino group is acylated by the next activated amino acid in the sequence. As will be readily appreciated, the reagents used to accomplish acylation are selected to create optimal reaction conditions and easy elimination of the excess reagents at the end of the coupling reaction. The activation for each acid coupling is performed with TBTU/HBTU and DIPEA. DIPEA is used as an additive to drive the reaction to completion. TBTU/HBTU and DIEA are dissolved in DMF: NMP (1:1) and added to the appropriate Fmoc protected amino acid to generate the acylation process on the free Nα-amino functionality that is attached to fully protected peptide being elongated while remaining attached to the modified Ramage resin.

During the deprotection step, the base-labile protecting group (Fmoc) is cleaved from the α-amino function of the N-terminal amino acid on the growing peptide chain by treating the resin twice with a 20% solution of piperidine in DMF (v/v). Typically, the resin is stirred for a period of 20 minutes after which the 20% piperidine/DMF solution is filtered, and the treatment is repeated for a second period of 20 minutes with a new 20% piperidine/DMF solution. This treatment is typically sufficient to allow complete cleavage of the Fmoc protecting group without affecting the stability of the acid-labile protecting groups present on the growing peptide chain.

In one example, synthesis of an AB-013 intermediate was performed using the tricyclic amide resin composed of a Ramage “connector” that is structurally defined as (9H-fluoren-9-yl)methyl-N-(6-{[(1-{[(4-methylphenyl) (phenyl)methyl]carbamoyl}pentyl)-carb-amoyl] meth-oxy}tricyclo[9.4.0.03,8] pentadecal (11), 3 (8),4,6,12,14-hexaen-2-yl) carb-amate bounded to the polystyrene supports cross-linked with 1% divinylbenzene (DVB). The Ramage “connector” was then modified to 2-[2-({2-aminotricyclo[9.4.0.03,8] pentadeca-1 (11),3 (8),4,6,12,14-hexaen-6-yl}oxy) acetamido]-N [(4methylphenyl) (phenyl)methyl] hexanamide (the modified Ramage resin), thus enabling the anchoring of the first amino acid on the solid support via standard coupling conditions.

The peptide synthesis methodology utilized a conventional Fmoc-protected amino acids approach with appropriately orthogonally protected amino acids. Coupling was achieved by using O-benzotriazol-1-yl-N,N,N′,N′-tetramethyl-uronium hexafluorophosphate (HBTU) and diisopropylethylamine (DIEA) as the activator mixture in N,N-dimethylformamide (DMF) solution. The Fmoc protective group was removed using 20% piperidine/DMF (v/v). A Boc-protected amino acid was used at the N-terminus of the peptide on solid support in order to generate a free Na-terminus after the AB-013 was cleaved from the resin. All amino acids used during the synthesis were L-amino acids, and sigma-coated glass reaction vessels were used during synthesis. As noted above, the synthesis process for the production of various peptide analogs and derivatives will vary widely, depending upon the nature of the various elements, i.e., the peptide analogs sequence, the linking group and the reactive entity, comprised in the peptide analogs or derivatives. In most circumstances, the chemically reactive coupling group (such as MPA or bromo-MPA) is coupled at the last stage of the synthesis. As will be appreciated, the chemically reactive coupling group should be placed at a site to allow the peptide to bond to the conformationally modified albumin while retaining its biological activity.

Peptide elongation on solid support: Solid phase peptide synthesis was carried out using the modified Ramage resin (substitution level 0.45 mmol/g) on a 100 μmole scale. The following protected amino acids were sequentially added to the resin: Fmoc-Lys40 (Aloc)-OH; Fmoc-Ser (tBu)-OH; Fmoc-Pro-OH; Fmoc-Pro-OH; Fmoc-Pro-OH; Fmoc-Ala-OH. (H2O); Fmoc-Gly-OH; Fmoc-Ser (tBu)-OH; Fmoc-Ser (tBu)-OH; Fmoc-Pro-OH; Fmoc-Gly-OH; Fmoc-Gly-OH; Fmoc-Asn (Trt)-OH; Fmoc-Lys (Boc)-OH; Fmoc-Leu-OH; Fmoc-Trp (Boc)-OH; Fmoc-Glu (OtBu)-OH (H2O); Fmoc-Ile-OH; Fmoc-Phe-OH; Fmoc-Leu-OH; Fmoc-Arg (Pmc)-OH; Fmoc-Val-OH; Fmoc-Ala-OH. (H2O); Fmoc-Glu (OtBu)-OH (H2O); Fmoc-Glu (OtBu)-OH (H2O); Fmoc-Glu (OtBu)-OH (H2O); Fmoc-Met-OH; Fmoc-Gln (Trt)-OH; Fmoc-Lys (Boc)-OH; Fmoc-Ser (tBu)-OH; Fmoc-Leu-OH; Fmoc-Asp (OtBu)-OH; Fmoc-Ser (tBu)-OH; Fmoc-Thr (tBu)-OH; Fmoc-Phe-OH; Fmoc-Thr (tBu)-OH; Fmoc-Gly-OH; Fmoc-Glu (OtBu)-OH (H2O); Fmoc-Gly-OH; Boc-His1 (Boc)-OH.

A molar excess of Fmoc-amino acid/HBTU/DIEA was used for each coupling of amino acids (3-5 eq.), which were dissolved in N,N-dimethylformamide (DMF) and, according to the sequence, activated using O-benzotriazol-1-yl-N,N,N′, N′-tetramethyl-uronium hexafluoro-phosphate (HBTU) and diisopropylethylamine (DIEA). Removal of the Fmoc protecting group was achieved using a solution of 20% piperidine/DMF for 20 minutes. After each coupling and/or Fmoc removal, the modified Ramage resin underwent washing cycles of 3× DMF followed by 3× isopropanol. This was repeated 3 more times, and the final wash was 1× DMF to swell the resin and prepare it for the next chemical step. It should be noted that the first amino acid anchored on the solid support can also be be Fmoc-Lys (Mtt)-OH or Fmoc-Lys (ivDDE)-OH as an alternative to Fmoc-Lys (Alloc)-OH or any Na-Fmoc-amino acid with a suitably protected orthogonal functionality that could lead to perform orthogonal chemistries of incorporating linker(s) such as AEEA and a reactive moiety to maleimidopropionic acyl (MPA).

Removal of the orthogonal protecting group on the first amino acid anchored on the solid support followed coupling of the linker AEEA and reactive moiety MPA: Orthogonal chemistries were performed selectively on the first amino acid anchored to the modified Ramage resin. Hence the orthogonal protecting group Alloc on the Lys40 was removed with 0.2 eq. tetrakis (triphenylphosphine) palladium (0) (Pd (PPh3) 4) and 2 eq. tributyltin-hydride (Bu3SnH) in dry DCM for 1 h. The resin underwent washing cycles of 3× DMF followed by 3λ isopropanol. This was repeated 3 more times, and the final wash was 1× DMF to swell the resin and prepare it for the next chemical step. The free amino functionality on the side chain of the Lys40 directly anchored on the modified Ramage resin was then acylated with Fmoc-AEEA-COOH under standard HBTU/DIEA coupling condition, which was followed by a standard washing cycle. Then, the Fmoc protecting the AEEA hydrophilic linker was removed using 20% piperidine/DMF for 20 minutes followed by a standard washing cycle. The free terminal amino moiety of the AEEA was subsequently acylated under standard HBTU/DIEA coupling conditions to yield the AB-013 intermediate.

Cleavage of the AB-013 intermediate from the resin and removal of all orthogonal protecting groups: The AB-013 intermediate was cleaved from the resin using 85% trifluoroacetic acid (TFA)/5% triisopropylsilane (TIS)/5% thioanisole and 5% phenol, followed by precipitation using ice cold (0-4° C.)ethyl ether (Et20). The crude peptide was collected on a polypropylene sintered funnel, dried, redissolved in a 40% mixture of acetonitrile (CH3CN) in water (0.1% TFA) and lyophilized to generate the corresponding crude material utilized in the purification process.

Purification: The AB-013 intermediate was purified by preparative reversed phase HPLC preparative binary HPLC system. The purification was performed using a Phenomenex Luna 10μ phenyl-hexyl, 50 mm×250 mm column (10μ) equilibrated with a water/TFA mixture (0.1% TFA in H20 (solvent A) and acetonitrile/TFA (0.1% TFA in CH3CN (solvent B). Elution was achieved at 50 mL/min by running a 28-38% B gradient over 180 min. Fractions containing peptide were detected by UV absorbance at 214 and 254 nm. The fractions were collected in 25 mL aliquots. Molecular weights of peptides are determined using Quadrupole Electro Spray mass spectroscopy. Those containing the desired product were identified by liquid-chromatography mass spectrometry (LC-MS). The selected fractions were subsequently analyzed by analytical HPLC (20-60% B over 20 min; Phenomenex Luna 5μ phenylhexyl, 10 mm×250 mm column, 0.5 mL/min) to identify fractions with >90% purity for pooling. The pooled fractions were homogenized, freeze-dried using liquid nitrogen and subsequently lyophilized for 2-3 days to yield a white amorphous powder.

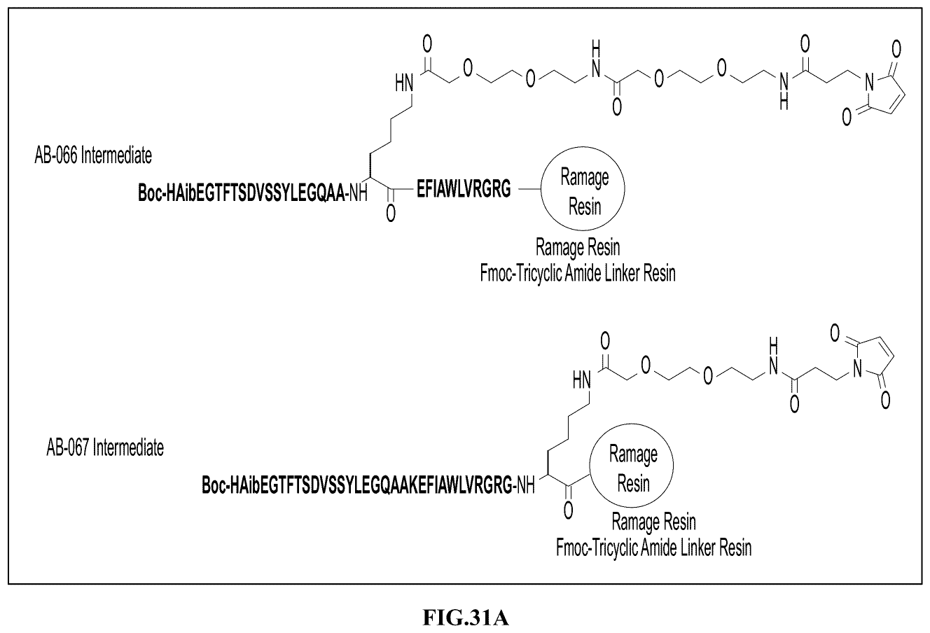

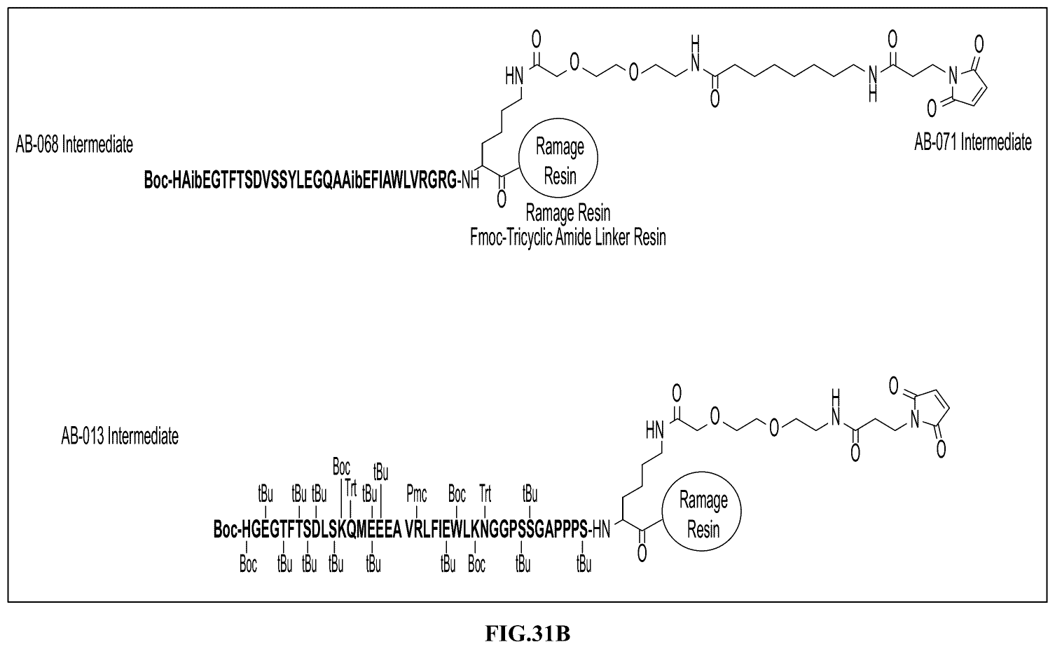

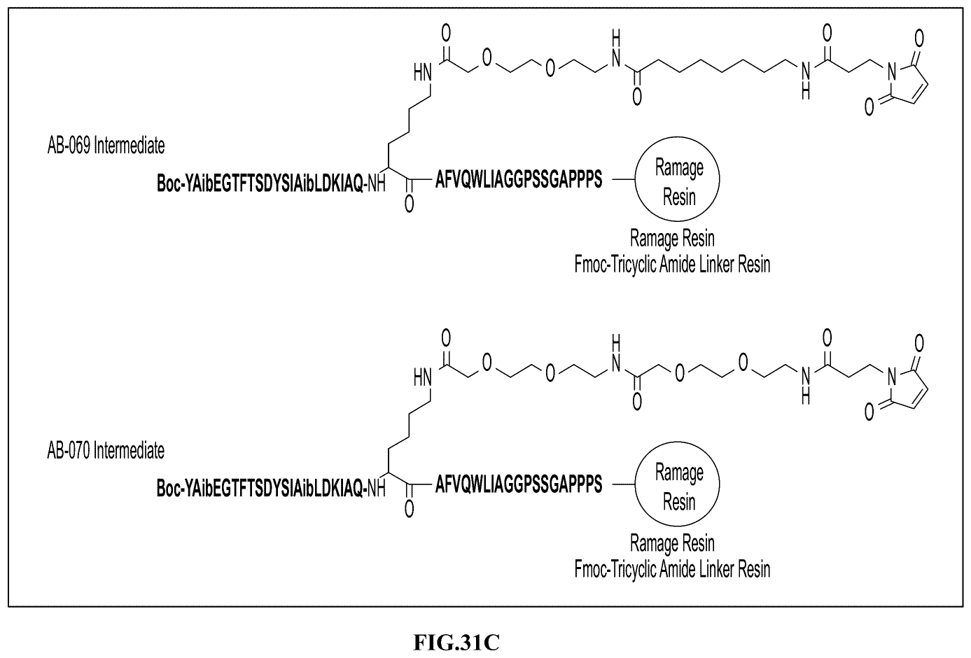

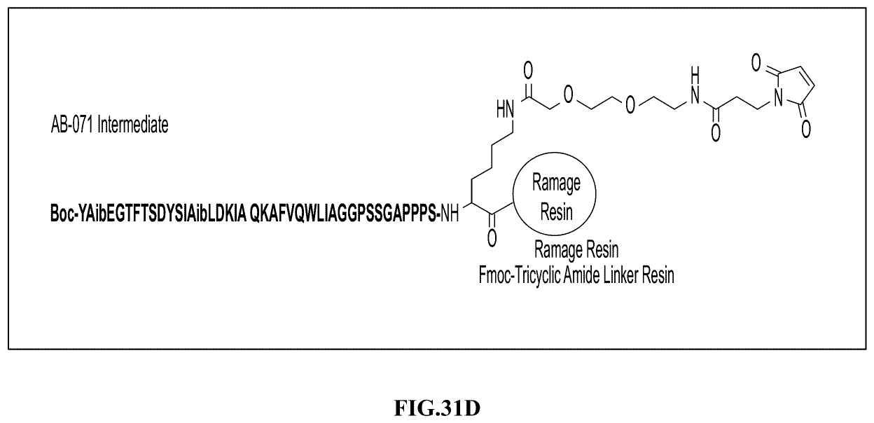

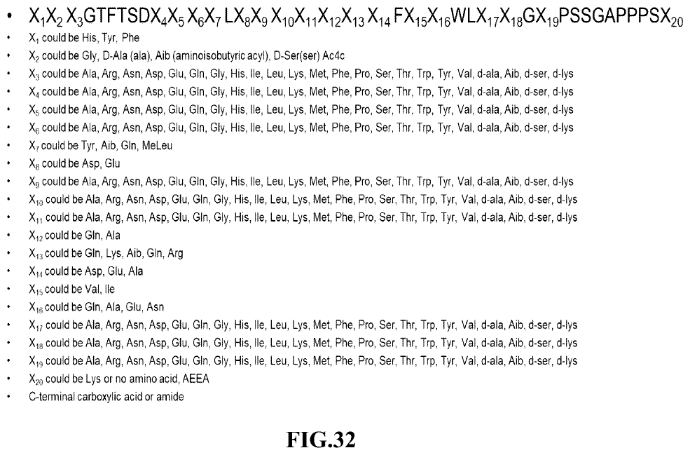

Of course, it should be appreciated that while the above examples and considerations are predominantly directed to linear peptide synthesis in which the reactive coupling group is covalently bound to the epsilon amino group of the C-terminal lysine amino acid, it should also be recognized that the reactive coupling group may also be covalently bound to the epsilon amino group of a lysine amino acid in a position other than a terminal position (e.g., at a position between the second and tenth amino acid, or at a position between the eleventh and twentieth amino acid, or at a position between the twenty first and thirtieth amino acid, or at a position between the thirty first and thirty ninth amino acid. FIG. 31 depicts exemplary structures of peptide agonist intermediates bound to a Ramage or Rink resin, and with protected intermediate for AB-013. Most typically, where the linker and reactive coupling group is at the C-terminal end of the agonist peptide, a Ramage resin is typically preferred. On the other hand, where the linker and reactive coupling group is coupled t the agonist in an intermediate position (and typically beyond the tryptophan cage where present) a Rink (amide) resin can be employed. As will be readily recognized, all resin-bound intermediates may be fully protected, or at least partially deprotected. Therefore, exemplary contemplated agonist peptide intermediates may have a sequence of SEQ ID NO:322, which is generically and schematically illustrated in FIG. 32. When bound to a resin, the intermediates may therefore also have a sequence of SEQ ID NO:322 and SEQ ID NO:323 shown below, with variable amino acids as shown in FIG. 32 and listed in the sequence listing.

SEQ ID NO: 322
X₁X₂X₃GTFTSDX₄X₅X₆X₇LX₈X₉X₁₀X₁₁X₁₂X₁₃X₁₄FX₁₅
X₁₆WLX₁₇X₁₈GX₁₉PSSGAPPPSX₂₀-Ramage Resin
SEQ ID NO: 323
X₁X₂X₃GTFTSDX₄X₅X₆X₇LX₈X₉X₁₀X₁₁X₁₂X₁₃X₁₄FX₁₅
X₁₆WLX₁₇X₁₈GX₁₉PSSGAPPPS-Rink Resin

Furthermore, it should be recognized that the agonist peptides may be synthesized in a single linear Merrifield synthesis process, or in a synthetic process in which two or more sub-sequences are condensed to a final sequence product. For example, a first peptide subsequence SEQ ID NO:324 may be coupled to a Ramage or Rink resin, while protected second and third subsequences SEQ ID NO:325 and SEQ ID NO:326 are prepared on respective chlorotrityl resins as shown below with variable amino acids as shown in FIG. 32.

SEQ ID NO: 324
NH₂X₁₉PSSGAPPPSX₂₀-Ramage Resin
SEQ ID NO: 325
Fmoc-X₁₁X₁₂X₁₃X₁₄FX₁₅X₁₆WLX₁₇
X₁₈G-Chlorotrityl Resin
SEQ ID NO: 326
Boc-X₁X₂X₃GTFTSDX₄X₅X₆X₇LX₈X₉
X₁₀-Chlorotrityl Resin

After decoupling from the resin of the second and third subsequences (resulting in N-terminal protected SEQ ID NO:325 and SEQ ID NO:326 having an unprotected C-terminus, respectively,

SEQ ID NO: 325
Fmoc-X₁₁X₁₂X₁₃X₁₄FX₁₅X₁₆WLX₁₇X₁₈G-COOH
SEQ ID NO: 326
Boc-X₁X₂X₃GTFTSDX₄X₅X₆X-LX₈X₉X₁₀-COOH

the second subsequence SEQ ID NO:325 is condensed with the first subsequence SEQ ID NO: 324 to yield resin-bound SEQ ID NO:328 shown below. Of course, it should be appreciated that other first peptide subsequences are also deemed suitable, and an exemplary subsequence is SEQ ID NO:327, bound to a Rink resin.

SEQ ID NO: 328
Fmoc-X₁₁X₁₂X₁₃X₁₄FX₁₅X₁₆WLX₁₇X₁₈G-
X₁₉PSSGAPPPSX₂₀-Ramage Resin

While the reaction product remains on the resin, the N-terminal protecting group is removed, resulting in deprotected SEQ ID NO:328 with a free NH₂ group at the N-terminus,

SEQ ID NO: 328
NH₂-X₁₁X₁₂X₁₃X₁₄FX₁₅X₁₆WLX₁₇X₁₈G-X₁₉
PSSGAPPPSX₂₀-Ramage Resin

to which the third deprotected subsequence SEQ ID NO:326 with deprotected C-terminus is coupled, resulting in the final resin-bound SEQ ID NO:329 with variable amino acids as shown in FIG. 32.

SEQ ID NO: 329
Boc-X₁X₂X₃GTFTSDX₄X₅X₆X₇LX₈X₉X₁₀-X₁₁
X₁₂X₁₃X₁₄FX₁₅X₁₆WLX₁₇X₁₈G-X₁₉PSSGAPP
PSX₂₀-Ramage Resin

In view of the above, it should therefore be appreciated that peptide solid state synthesis intermediates can be readily prepared that include a Ramage resin to which a first amino acid of a peptide is covalently coupled at a C-terminus of the peptide via an amide bond, wherein the first amino acid is further covalently coupled to a linker via an amide bond in a side chain of the first amino acid, and wherein the linker comprises a reactive coupling group.

As already noted above, agonists with a linker and reactive coupling group may also prepared where the linker and reactive coupling group is in an intermediate position in the agonist peptide. For example, synthesis of an intermediate with a reactive coupling group will typically follow known methodologies (see e.g., Stewart et al. in “Solid Phase Peptide Synthesis”, W. H. Freeman Co. (San Francisco), 1963 and Meienhofer, Hormonal Proteins and Peptides, 1973, 2 46. For classical solution synthesis, see for example Schroder et al. in “The Peptides”, volume 1, Academic Press (New York)). Such methodologies typically comprise the sequential addition of one or more amino acids or suitably protected amino acids to a growing peptide chain. Normally, either the amino or carboxyl group of the first amino acid is protected by a suitable protecting group. The protected or derivatized amino acid is then either attached to an inert solid support or utilized in solution by adding the next amino acid in the sequence having the complementary (amino or carboxyl) group suitably protected and under conditions suitable for forming the amide linkage. The protecting group is then removed from this newly added amino acid residue and the next amino acid (suitably protected) is added, and so forth. After all the desired amino acids have been linked in the proper sequence, any remaining protecting groups (and any solid support) are removed sequentially or concurrently to afford the final polypeptide. By simple modification of this general procedure, it is possible to add more than one amino 5 acid at a time to a growing chain, for example, by coupling (under conditions which do not racemize chiral centers) a protected tripeptide with a properly protected dipeptide to form, after deprotection, a pentapeptide.

Using the solid support 4-(2′,4′-Dimethoxyphenyl-Fmoc-aminomethyl) phenoxy-acetamido MBHA resin (Rink amide MBHA resin), the Fmoc group is cleaved with a secondary amine, preferably piperidine in DMF (20% in DMF v: v), prior to coupling with the α-C-terminal amino acid. A method for coupling to the deprotected 4-(2′,4′-Dimethoxyphenyl-Fmoc-aminomethyl) phenoxyacetamido MBHA resin is O-benzotriazol-1-yIN,N,N′,N′-tetramethyluroniurnhexafluoro-phosphate (HBTU), diisopropylethylamine (DIEA), and optionally 1-hydroxybenzotriazole (HOBT), in DMF. The coupling of successive protected amino acids can be carried out in an automatic and/or semi-synthetic and/or manual peptide synthesizer in a conventional manner as is well known in the art. The removal of the Fmoc protecting group from the αN-terminal side of the growing peptide is accomplished conventionally, for example, by treatment with a secondary amine, preferably piperidine in DMF (20% v: v). Each protected amino acid is then introduced in 1-to-5-fold molar excess, and the coupling is preferably carried out in DMF and/or NMP. At the end of the peptide elongation on solid support and addition of the orthogonal functionalities such as linker(s) and a reactive moiety to the fully protected peptide it is removed from the resin and deprotected, either in successive operations or in a single operation. Removal of the polypeptide and deprotection can be accomplished conventionally in a single operation by treating the resin-bound polypeptide with a cleavage reagent comprising thioanisole, triisopropylsilane, phenol, and trifluoroacetic acid. In cases wherein the a-C-terminal of the polypeptide is an alkylamide, the resin is cleaved by aminolysis with an alkylamine. Alternatively, the peptide may be removed by transesterification, e.g., with methanol, followed by aminolysis or by direct transamidation. The protected peptide may be purified at this point or taken to the next step directly. The removal of the side chain protecting groups is accomplished using the cleavage mixture described above.

The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (such as Amberlite XAD™); silica gel adsorption chromatography; ion exchange chromatography on carboxymethylcellulose; partition chromatography, e.g., on Sephadex G-25™, LH-20™ or countercurrent distribution; high performance liquid chromatography (HPLC), especially reverse-phase HPLC on octyl- or octadecylsilylsilica bonded phase column packing. Anyone of ordinary skill in the art will be able to determine easily what would be the preferred chromatographic steps or sequences required to obtain acceptable purification of the peptide analogs.

For example, small scale peptide synthesis of the AB-092 intermediate can be performed as follows.

Peptide elongation on solid support: Solid phase peptide synthesis was carried out using the modified Rink MBHA resin (substitution level 0.5 mmol/g) on a 100 μmole scale. The following protected amino acids were sequentially added to resin: Fmoc-Ser39 (tBu)-OH; Fmoc-Pro-OH; Fmoc-Pro-OH; Fmoc-Pro-OH; Fmoc-Ala-OH. (H₂O); Fmoc-Gly-OH; Fmoc-Ser (tBu)-OH; Fmoc-Ser (tBu)-OH; Fmoc-Pro-OH; Fmoc-Gly-OH; Fmoc-Gly-OH; Fmoc-Glu (OtBu)-OH (H₂O); Fmoc-Leu-OH; Fmoc-Leu-OH; Fmoc-Tyr (OtBu)-OH; Fmoc-Glu (OtBu)-OH (H₂O); Fmoc-Ile-OH; Fmoc-Phe-OH; Fmoc-Ala-OH. (H₂O); Fmoc-Aib-OH; Fmoc-Gln (Trt)-OH; Fmoc-Ala-OH (H₂O); Fmoc-Lys (Alloc)-OH; Fmoc-Lys (Boc)-OH; Fmoc-Asp (OtBu)-OH; Fmoc-Leu-OH; Fmoc-aMetyl-Leu-OH; Fmoc-Ile-OH; Fmoc-Ser (tBu)-OH; Fmoc-Tyr (OtBu)-OH; Fmoc-Asp (OtBu)-OH; Fmoc-Ser (tBu)-OH; Fmoc-Thr (tBu)-OH; Fmoc-Phe-OH; Fmoc-Thr (tBu)-OH; Fmoc-Gly-OH; Fmoc-Gln (Trt)-OH of Fmoc-amino (H2O); Fmoc-Aib-OH; Boc-Tyrl(OtBu)-OH. A molar excess of acid/HBTU/DIEA was used for each coupling of amino acids (3-5 eq.). They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% piperidine/DMF for 20 minutes. After each coupling and/or Fmoc removal the modified Ramage resin underwent washing cycles of 3× DMF followed by 3× Isopropanol. This was repeated 3 more times, and the final wash was 1× DMF to swell the resin and prepare it for the next chemical step. Alternatively, the first amino acid anchored on the solid support can be Fmoc-Lys (Mtt)-OH or Fmoc-Lys (ivDDE)-OH as an alternative to Fmoc-Lys (Alloc)-OH or any Na-Fmoc-amino acid with a suitably protected orthogonal functionality that could lead to perform orthogonal chemistries of incorporating linker(s) such as but not limited to AEEA and a reactive moiety such as but not limited to maleimidopropionic acyl (MPA).

Removal of the orthogonal protecting group on the first amino acid anchored on the solid support followed coupling of the linker AEEA twice and reactive moiety MPA. Orthogonal chemistries were performed selectively on the first amino acid anchored to the Rink amide MBHA resin. Hence the orthogonal protecting group Alloc on the Lys17 was removed with 0.2 eq. tetrakis (triphenylphosphine) palladium (0) (Pd (PPh3) 4) and 2 eq. tributyltin-hydride (Bu₃SnH) in dry DCM for 1 h. The resin underwent washing cycles of 3× DMF followed by 3× Isopropanol. This was repeated 3 more time, and the final wash was 1× DMF to swell the resin and prepare it for the next chemical step. The free amino functionality on the side chain of the Lys₁₇ directly anchored on the Rink amide MBHA resin was acylated with Fmoc-AEEA-COOH (twice) under standard HBTU/DIEA coupling condition. It was then followed by a standard washing cycle. Then, the Fmoc protecting the AEEA hydrophilic linker was removed using 20% piperidine/DMF for 20 minutes followed by a standard washing cycle. The free terminal amino moiety of the second AEEA was then acylated with under standard HBTU/DIEA coupling condition.

Cleavage of the AB-092 intermediate from the resin and removal of all orthogonal protecting groups: The AB-092 intermediate was cleaved from the resin using 85% trifluoroacetic acid (TFA)/5% triisopropylsilane (TIS)/5% thioanisole and 5% phenol, followed by precipitation using ice cold (0-4° C.) Ether (Et20). The crude peptide was collected on a polypropylene sintered funnel, dried, redissolved in a 40% mixture of acetonitrile (CH3CN) in water (0.1% TFA) and lyophilized to generate the corresponding crude material utilized in the purification process.

Purification protocol for small scale synthesis: The AB-092 intermediate was purified by preparative reversed phase HPLC preparative binary HPLC system. The purification was performed using a Phenomenex Luna 10μ phenyl-hexyl, 50 mm×250 mm column (10μ) equilibrated with a water/TFA mixture (0.1% TFA in H₂0 (solvent A) and acetonitrile/TFA (0.1% TFA in CH₃CN (solvent B). Elution was achieved at 50 mL/min by running a 28-38% B gradient over 180 min. Fractions containing peptide were detected by UV absorbance at 214 and 254 nm. The fractions were collected in 25 mL aliquots. Molecular weights of peptides are determined using Quadrupole Electro Spray mass spectroscopy. Those containing the desired product were identified by liquid-chromatography mass spectrometry (LC-MS). The selected fractions were subsequently analyzed by analytical HPLC (20-60% B over 20 min; Phenomenex Luna 5μ phenylhexyl, 10 mm×250 mm column, 0.5 mL/min) to identify fractions with >90% purity for pooling. The pooled fractions were homogenized, freeze-dried using liquid nitrogen and subsequently lyophilized for at least 2-3 days to yield a white amorphous powder.

In view of the above, it should therefore be appreciated that contemplated resin-immobilized cysteine-reactive Class B GPCR agonist peptides will include a GPCR agonist peptide comprising a Lys amino acid to which (a) a linker is covalently bound via an amide bond formed by an epsilon amino group of the Lys amino acid, and (b) a synthetic resin with a spacer is covalently bound via an amide bond formed by a distal amino group in the spacer. With respect to suitable GPCR agonist peptides, linker, and resins, the same considerations as provided above apply.

With respect to suitable resin beads it is generally known that reaction kinetics are generally faster using smaller beads due to the higher surface area to volume ratio. In practice, however, too small a bead can lead to extended filtration times. Therefore, suitable resin beads will have a size in the range of 75 to 150 microns in diameter offer a good balance of reaction kinetics versus reliability or 35-75 microns respectively. Typically, the resins will have a substitution level varying from 0.1 to 2.0 mmol/g, and more preferably between 0.4 and 0.6 mmol/g.

It should further be appreciated that peptides have either a carboxylic acid group (—COOH) or an amide group (—CONH₂) at their C-terminus. Therefore, common resins for preparing peptide amides include MBHA, BHA, Rink amide, Knorr, DCHD, PAL, Seiber, and Ramage (tricyclic amide) resins. Alternatively, and especially where smaller peptides with less steric demand are prepared, suitable resins include Merrifield, hydroxymethyl polystyrene, Wang, and 2-chlorotrityl resins. Among other factors, suitable resins can be selected based on the following considerations: aminomethyl (AM) resins have long been used in solid phase peptide synthesis as a core resin to which various linkers could be attached through a stable amide bond. 4-Methylbenzhydryl amine (MBHA) resins, however, were originally developed for the formation of peptide amides using the Boc-N protection/TFA deprotection strategy. These resins form very stable amide or amine linkages to either carboxylic or electrophilic alkyl substrates. Generally, strong acid conditions are required to cleave substrates from these resins. Solid phase supports for the formation of amide products including Rink and Ramage resins were originally developed for peptide amide synthesis using the Fmoc strategy. These resins are favored due to their higher acid lability; cleavage can be performed under conditions as mild as 1% TFA. The Sieber resin is useful for preparing amides and amines and fully protected peptide amide fragments. Products can be cleaved under mild conditions using 1% TFA in DCM. This resin is less sterically hindered than Rink resin and thus allows for higher loading in sterically demanding applications than Rink resins. MBHA (methylbenzhydryl amine) is an amide-forming resin structurally similar to Rink however conditions for cleaving peptide products from MBHA are much harsher than from Rink as MBHA requires treatment with hydrofluoric acid or trifluoromethanesulfonic acid (TFMSA). Trityl and 2-Chlorotrityl resins have been widely used in peptide chemistry. These resins are very acid labile and can be cleaved with acetic acid and protected peptides can be cleaved with 1:4 v/v hexafluoroisopropyl alcohol/dichloromethane with all sidechain protecting groups intact, even trityl groups on sulfhydryl function of homocysteine. The Trityl and 2-Chlorotrityl resins are particularly useful when less acid labile protecting groups are required on the substrate following cleavage resulting in fully protected short peptides and/or peptide fragments/components capable of undergoing a hybrid manufacturing process leading to the desired larger peptide structure.

In view of the above, preferred resins for the solid phase synthesis of peptide intermediates will therefore include MBHA, BHA, Rink amide, Knorr, DCHD, PAL, Seiber, and Ramage (tricyclic amide) resins, and especially a Ramage (tricyclic amide) resin. Moreover, preferred resins will be about 1% cross-linked, with a size of between 200-400 Mesh.

As will be further appreciated, the peptide intermediates presented herein can be produced using one of three synthesis methods: solid phase peptide synthesis (SPPS), liquid phase peptide synthesis (LPPS) or a hybrid approach. LPPS could produce large quantities of high-quality short peptides and/or peptide fragments/components of a larger peptide structure. Alternatively, SPPS could provide peptides of longer lengths but, depending on the structure, could have diminished yields typically due to challenges in removing closely related impurities from the desired product. A hybrid approach could bring the SPPS and LPPS methodologies together to produce peptides. For example, to construct a 40-amino acid peptide intermediate, small peptides of 10 to 15 amino acid segments would be produced using SPPS and then the segment condensations would occur in LPPS to construct the full peptide intermediate sequence as exemplarily shown above.

Thus, and in view of the above, it should be appreciated that Class B GPCR agonist fusion proteins can be readily prepared by providing or producing a conformationally modified albumin as described above and by covalently coupling a GPCR agonist peptide to Cys₃₄ of the conformationally modified albumin via a Michael addition reaction, typically at a pH of pH<7.0. With respect to the GPCR agonist peptide, the linker, the reactive coupling group, and the albumin, the same considerations as provided above apply.

For example, especially suitable agonist peptides are provided in Table 1 below in which the amino acids and modifying characters are as provided in the sequence listing of this application. Moreover, it should be appreciated that (1) sequences in the table without linker modifications may also include a linker modification as described herein, particularly where the linker modification is covalently attached to an epsilon amino group of a lysine, and suitable linker modifications include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA; (2) any specific linker modification shown in the table below (e.g., AEEA-MPA) as indicated by a particular modifying character may be replaced with any other linker modification contemplated herein (e.g., AEEA-AEEA-MPA); (3) the C-terminal amino acid may be covalently attached to a solid phase (e.g., modified Ramage resin), preferably via a spacer group; (4) any sequence in the table below may be covalently coupled via a linker as described herein to albumin at Cys₃₄, preferably in a retro-Michael resistant manner (e.g., in a stereopreferred or stereoselective manner); and (5) any sequence in the table below having a linker modification is also contemplated as an agonist peptide sequence without the linker modification.

| SEQ ID | |
|---|---|
| NO: | SEQUENCE |
| 1 | HaEGTFTSDVSSYLEGQAAKEFIAWLVKGRK* |
| 2 | HGEGTFTSDLSKQMEEEAVRLFIEWLKNGGPSSGAPPPSK* |
| | (Albenatide-AB013 when |
| | bound to Cys₃₄ in retro-Michael |
| | resistant/stereopreferred or |
| | stereoselective manner via |
| | maleimide reactive group) |
| 3 | HGEGTFTSDLSKQMEEEAVRLFIEWLKNGGPSSGAPPPS |
| 4 | HAEGTFTSDVSSYLEGQAAKEFIAWLVKGR |
| 5 | YAEGTFISDYSIAMDKIHQQDFVNWLLAQKGKKNDWKHNITQ |
| 6 | HSQGTFTSDYSKYLDSRRAQDFVQWLMNT |
| 7 | HaEGTFTSDVSSYLEGQAAKEFIAWLVNGGPSSGAPPPSK* |
| | (Albugiptide-AB-029 when |
| | bound to Cys₃₄ in retro-Michael |
| | resistant/stereopreferred or |
| | stereoselective manner via |
| | maleimide reactive group) |
| 8 | HaEGTFTSDYAKYLDARRAKEFIAWLVKGRPSSGAPPPSK* |
| 9 | HGEGTFTSDLSKQMEEEAVRLFIEWLKNGGPK*SGAPPPS |
| 10 | HaEGTFTSDVSK*YLEGQAAKEFIAWLVKGRPSSGAPPPS |
| 11 | HaEGTFTSDVSSYLEK*QAAKEFIAWLVKGRPSSGAPPPS |
| 12 | HGEGTFTSDLSKQMEEEAVRLFIEWLKNGGPK*SGAPPPS |
| 13 | HaEGTFTSDINKVLDTIAAKEFIAWLVKGRPSSGAPPPSK* |
| 14 | YAibEGTFTSDYSIYLDKQAAAibEFVQWLLAGGPSSGAPPPSK″ |
| 15 | YGGGTFTSDSFFYLELSHAKDFINWLQLGaPSSGAPPPSK* |
| 16 | HAibAibGTFTSDEMNYLDDWMQAibAFVNWLVAibGIPS |
| | SGAPPPSK* |
| 17 | FAibPGTFTSDGHNYLDWQDAKEFIQWLGWGVPSSGAPPPSK* |
| 18 | YAibMGTFTSDPQIAibLEMKEQAibDFINWLNDGFPSSGAPPPSK* |
| 19 | HaPGTFTSDNHEAibLDTYRQAibDFINWLDTGVPSSGAPPPSK* |
| 20 | YaNGTFTSDIWAYLDSSFAQDFVAWLYIGKPSSGAPPPSK* |
| 21 | HAibDGTFTSDFEHYLEDAVQKAFIAWLSTGVPSSGAPPPSK* |
| 22 | FGDGTFTSDLRGAibLEIMPQAibAFVNWLTSGKPSSGAPPPSK* |
| 23 | HaaGTFTSDsMIYLDNAHQQEFIQWLFNGPPSSGAPPPSK* |
| 24 | HaIGTFTSDsTAibYLEaYKQAibDFVQWLHSGGPSSGAPPPSK* |
| 25 | YsWGTFTSDaGYYLDMsFQQAFIQWLKAGaPSSGAPPPSK* |
| 26 | YGRGTFTSDWIYAibLDTaPQKEFVEWLHsGAPSSGAPPPSK* |
| 27 | YaMGTFTSDSEEAibLEMVYQQAFVAWLPVGGPSSGAPPPSK* |
| 28 | FssGTFTSDWDWAibLDIAibIAAibEFINWLVYGYPSSGAPPPSK* |
| 29 | FAibGGTFTSDDMRYLEPKGQQAFIQWLWVGQPSSGAPPPSK* |
| 30 | YAibSGTFTSDVETYLDLLIAKEFIAWLGAGAibPSSGAPPPSK* |
| 31 | HGRGTFTSDHQFAibLDSMIQAibEFIQWLHYGIPSSGAPPPSK* |
| 32 | YaRGTFTSDFINAibLEsGVAKAFVNWLQEGaPSSGAPPPSK* |
| 33 | HsFGTFTSDGWMYLDMNEQKAFVAWLYaGaPSSGAPPPSK* |
| 34 | FsRGTFTSDHYRAibLDQSKAAibEFIEWLESGNPSSGAPPPSK* |
| 35 | FsGGTFTSDHHRYLDQIPQKDFIQWLEFGKPSSGAPPPSK* |
| 36 | FaQGTFTSDaNMAibLDMFaQKAFINWLFQGGPSSGAPPPSK* |
| 37 | FaAGTFTSDVTaAibLEVAibQQKAFIAWLNLGQPSSGAPPPSK* |
| 38 | HaIGTFTSDHMAibYLDsDPAQDFIEWLMPGSPSSGAPPPSK* |
| 39 | HAibNGTFTSDIIHYLDPIVQKAFIEWLNGGAPSSGAPPPSK* |
| 40 | HGEGTFTSDMAibQAibLDREIAKEFVEWLFFGYPSSGAPPPSK* |
| 41 | HsPGTFTSDPDsAibLDENHQQAFVAWLNsGMPSSGAPPPSK* |
| 42 | FsaGTFTSDsLDYLEssEQAibEFINWLAAGVPSSGAPPPSK* |
| 43 | YaAibGTFTSDKDPYLEAibTYAAibEFVAWLVsGRPSSGAPPPSK* |
| 44 | FGRGTFTSDAVMAibLDAMQAKAFIEWLWIGIPSSGAPPPSK* |
| 45 | HaaGTFTSDVDYAibLEWKIQQDFIEWLSTGLPSSGAPPPSK* |
| 46 | YsFGTFTSDIAGYLDLEAibAAibAFVQWLAHGKPSSGAPPPSK* |
| 47 | HsHGTFTSDEVQYLDMDGQQEFINWLDLGYPSSGAPPPSK* |
| 48 | HsRGTFTSDLQGAibLEYADQAibDFVNWLAibTGFPSSGAPPPSK* |
| 49 | HGFGTFTSDGIDAibLEaITAKEFIAWLaIGKPSSGAPPPSK* |
| 50 | HAibTGTFTSDIalAibLDYKGAAibAFVQWLDAGEPSSGAPPPSK* |
| 51 | HsLGTFTSDLAibMAibLDVNIAAibDFIAWLMIGYPSSGAPPPSK* |
| 52 | YGDGTFTSDKHRAibLEISQQKEFVQWLLsGLPSSGAPPPSK* |
| 53 | FaaGTFTSDQTTAibLDAGSAKDFVNWLVAibGAibPSSGAPPPSK* |
| 54 | YAibGGTFTSDEPPYLDAWTQAibEFVEWLVRGWPSSGAPPPSK* |
| 55 | HaGGTFTSDGSRAibLDIaTQQDFIEWLLEGHPSSGAPPPSK* |
| 56 | HsSGTFTSDPLKYLDHGNQQAFVNWLISGSPSSGAPPPSK* |
| 57 | HAibTGTFTSDISFYLEEYVAQEFVAWLQAGsPSSGAPPPSK* |
| 58 | HsaGTFTSDaYEAibLEPAibDQKAFIAWLWHGLPSSGAPPPSK* |
| 59 | YsRGTFTSDATLAibLEGSKQKAFINWLLEGIPSSGAPPPSK* |
| 60 | FGWGTFTSDLEKAibLELARAQAFVEWLKVGSPSSGAPPPSK* |
| 61 | FAibMGTFTSDPYPYLEWEaAQAFINWLsGGSPSSGAPPPSK* |
| 62 | HaAibGTFTSDMPTAibLDHIQQQDFVAWLVQGIPSSGAPPPSK* |
| 63 | FasGTFTSDHSDAibLELKAQQEFVNWLRNGRPSSGAPPPSK* |
| 64 | HGQGTFTSDYSKYLDARRAQDFVEWLKNGGPSSGAPPPSK* |
| 65 | HAibQGTFTSDYSKAibLDKRRAKDFVEWLKNGGPSSGAPPPSK* |
| 66 | HAibQGTFTSDYSKYLDKRRAKDFVEWLKNGGPSSGAPPPSK* |
| 67 | YAibQGTFTSDYSImeILDKIAQAibAFIEYLLEGGPSSGAPPPSK* |
| 68 | YAibEGTFTSDYSIAibLDKIAQKAFVQWLIAGGPSSGAPPPSK* |
| 69 | HaEGTFTSDYAKYLDARRAKEFIAWLVNGGPSSGAPPPSK* |
| 70 | YAibEGTFTSDYSIYLDKQAAAibEFVQWLLAGGPSSGAPPPSK* |
| 71 | HAibHGTFTSDLSKLLEEQRQAibEFIEWLKAaGPPPSAibKPPPK* |
| 72 | HaMGTFTSDRHWYLDMSHQK*AFVQWLAYGNPSSGAPPPS |
| 73 | YasGTFTSDWGRYLELLIQK*EFVNWLIIGAPSSGAPPPS |
| 74 | HGKGTFTSDsAIAibLEVsaAK*AFIEWLaHGDPSSGAPPPS |
| 75 | HasGTFTSDILEAibLDQAAAK*EFVEWLaHGFPSSGAPPPS |
| 76 | HaYGTFTSDYMAibAibLDFLQQK*DFVAWLFMGVPSSGAPPPS |
| 77 | HAibsGTFTSDAibAYYLDNTTAK*DFIQWLDAGPPSSGAPPPS |
| 78 | FAibKGTFTSDDaVYLEAibHRAK*EFVEWLFGGsPSSGAPPPS |
| 79 | YAibHGTFTSDLTVAibLDEIKAK*EFVEWLAibVGPPSSGAPPPS |
| 80 | FsWGTFTSDFPaAibLEAibMKAK*DFVEWLLDGNPSSGAPPPS |
| 81 | HAibKGTFTSDDADAibLEWYRQK*AFVQWLPsGIPSSGAPPPS |
| 82 | YAibWGTFTSDSNSAibLEAKMQK*EFVNWLsQGFPSSGAPPPS |
| 83 | FaTGTFTSDDADYLELFsQK*AFIAWLDNGVPSSGAPPPS |
| 84 | YSYGTFTSDWDAAibLESAibMQK*EFVQWLFYGQPSSGAPPPS |
| 85 | YaNGTFTSDTNsAibLDSTQAK*EFVAWLVQGDPSSGAPPPS |
| 86 | FAibIGTFTSDKQAYLEHPRQK*AFVAWLDVGYPSSGAPPPS |
| 87 | HAibRGTFTSDKFIAibLEYHNAK*EFVAWLYKGsPSSGAPPPS |
| 88 | FGHGTFTSDSIWYLENYSQK*EFIEWLEKGPPSSGAPPPS |
| 89 | HsaGTFTSDIRAibYLEIMLQK*EFVEWLaEGVPSSGAPPPS |
| 90 | HAibFGTFTSDMLNYLEENSQK*EFVNWLQLGMPSSGAPPPS |
| 91 | FGHGTFTSDIWIYLEVQTAK*DFINWLSWGEPSSGAPPPS |
| 92 | HsGGTFTSDSGPYLDKTDQK*AFINWLPIGNPSSGAPPPS |
| 93 | YSIGTFTSDHsPAibLDHLFAK*DFVEWLENGDPSSGAPPPS |
| 94 | FGYGTFTSDKEGAibLEQsAibAK*EFIQWLPHGPPSSGAPPPS |
| 95 | FsWGTFTSDFAibSAibLDTTRAK*DFVEWLIRGNPSSGAPPPS |
| 96 | HAibPGTFTSDHEKYLEMVSAK*DFIAWLRAibGDPSSGAPPPS |
| 97 | YaAGTFTSDTIaAibLDNaAAK*DFIAWLIQGYPSSGAPPPS |
| 98 | YAibLGTFTSDTIHAibLEFEAQK*DFINWLKAGEPSSGAPPPS |
| 99 | YAibQGTFTSDAMIYLDTPDAK*EFIAWLAibIGGPSSGAPPPS |
| 100 | YGAGTFTSDHAibGAibLDIAibQQK*EFVQWLETGTPSSGAPPPS |
| 101 | YaVGTFTSDLSEYLEMNIAK*DFVQWLLVGGPSSGAPPPS |
| 102 | YGPGTFTSDRYSYLEQHMAK*EFIQWLGPGHPSSGAPPPS |
| 103 | YAibRGTFTSDMRVYLEETLOK*AFVQWLIaGDPSSGAPPPS |
| 104 | YsAibGTFTSDFMEAibLDKKIQK*DFIQWLWYGSPSSGAPPPS |
| 105 | FAibsGTFTSDELRYLEEMHQK*AFIQWLAibTGFPSSGAPPPS |
| 106 | YGPGTFTSDWAKYLEGRDAK*AFINWLsQGSPSSGAPPPS |
| 107 | HGVGTFTSDHHFAibLEIIMAK*AFIEWLWHGaPSSGAPPPS |
| 108 | HGIGTFTSDVPAYLDPAaAK*DFIQWLVSGGPSSGAPPPS |
| 109 | HsNGTFTSDRRKAibLEPPIAK*EFVQWLIAibGIPSSGAPPPS |
| 110 | FAibTGTFTSDTMaYLDYTHAK*DFIAWLIDGKPSSGAPPPS |
| 111 | YaTGTFTSDsVQYLEHPMQK*AFVAWLHTGRPSSGAPPPS |
| 112 | YAibTGTFTSDEEWYLDNWMQK*AFVQWLSNGAibPSSGAPPPS |
| 113 | YSIGTFTSDGNKAibLEAibGRAK*AFIAWLQYGRPSSGAPPPS |
| 114 | YsPGTFTSDMYVYLEPGDAK*EFVQWLKNGGPSSGAPPPS |
| 115 | FsIGTFTSDLMaAibLERAAQK*EFVNWLIIGSPSSGAPPPS |
| 116 | YAibNGTFTSDIEQYLEPMVQK*EFVQWLSPGNPSSGAPPPS |
| 117 | YGWGTFTSDSFsAibLDWMMAK*AFIQWLIVGIPSSGAPPPS |
| 118 | HsPGTFTSDAibTHYLDNDQQK*DFVNWLPEGWPSSGAPPPS |
| 119 | YGaGTFTSDNHTAibLESFAAK*EFIQWLNSGQPSSGAPPPS |
| 120 | YaMGTFTSDRLRAibLDQFSAK*AFVNWLSWGEPSSGAPPPS |
| 121 | FAibaGTFTSDSLAibAibLDHHNQK*AFVEWLaPGLPSSGAPPPS |
| 122 | HGQGTFTSDYSKYLDK*RRAQDFVEWLKNGGPSSGAPPPS |
| 123 | HAibQGTFTSDYSKAibLDKRRAK*DFVEWLKNGGPSSGAPPPS |
| 124 | HAibQGTFTSDYSKYLDK*RRAQDFVEWLKNGGPSSGAPPPS |
| 125 | YAibEGTFTSDYSIAibLDKIAQK*AFVQWLIAGGPSSGAPPPS |
| 126 | YAibQGTFTSDYSImeILDKIAQK*AFIEYLLEGGPSSGAPPPS |
| 127 | HaEGTFTSDYAKYLDARRAK*EFIAWLVNGGPSSGAPPPS |
| 128 | YaWGTFTSDsKDYLEFK*WAAibEFIQWLDPGDPSSGAPPPS |
| 129 | FAibYGTFTSDINVAibLDIK*WAAibAFINWLPGGIPSSGAPPPS |
| 130 | FaaGTFTSDRQEAibLDVK*LAAibEFVQWLVSGDPSSGAPPPS |
| 131 | FaRGTFTSDSMQAibLETK*RQQEFVNWLsMGVPSSGAPPPS |
| 132 | YaHGTFTSDTNRAibLESK*AAAibEFIAWLEVGAPSSGAPPPS |
| 133 | FAibGGTFTSDFRsAibLDsK*IAAibEFINWLFKGFPSSGAPPPS |
| 134 | YAibNGTFTSDMMPYLEPK*HQKDFVEWLTSGDPSSGAPPPS |
| 135 | HaPGTFTSDIIHYLETK*IAAibEFIQWLKRGSPSSGAPPPS |
| 136 | FsVGTFTSDAibSAAibLDLK*IAAibEFINWLFaGFPSSGAPPPS |
| 137 | YAibaGTFTSDGNMYLDKK*HAKDFIEWLsSGPPSSGAPPPS |
| 138 | YaIGTFTSDPsHAibLEVK*NQKEFVQWLNRGIPSSGAPPPS |
| 139 | FsTGTFTSDFVEAibLEIK*EQKAFIEWLAQGGPSSGAPPPS |
| 140 | FGTGTFTSDFDGYLEDK*aAKAFIQWLsGGMPSSGAPPPS |
| 141 | HsWGTFTSDWYIAibLEEK*NAAibDFIAWLYAGYPSSGAPPPS |
| 142 | FGPGTFTSDKKFAibLELK*GAKAFIAWLAibAibGFPSSGAPPPS |
| 143 | HsNGTFTSDaNsYLEPK*AAAibDFVNWLDAibGWPSSGAPPPS |
| 144 | FsTGTFTSDPWVYLDSK*QQQAFVEWLHFGNPSSGAPPPS |
| 145 | FaEGTFTSDWYPAibLDLK*SAAibDFVQWLYGGPPSSGAPPPS |
| 146 | FGIGTFTSDTARAibLDDK*DQKEFVQWLNDGMPSSGAPPPS |
| 147 | FGYGTFTSDKSTYLDEK*IQAibDFVEWLNDGsPSSGAPPPS |
| 148 | HGSGTFTSDAAibPYLDQK*GAQDFIAWLDGGPPSSGAPPPS |
| 149 | HaIGTFTSDPPVAibLEQK*HQQEFVAWLDPGLPSSGAPPPS |
| 150 | YaYGTFTSDVNKYLDPK*TAAibAFVEWLaQGIPSSGAPPPS |
| 151 | FaIGTFTSDRREYLEPK*EQAibDFIEWLRDGGPSSGAPPPS |
| 152 | YGNGTFTSDAWIYLDDK*LQKEFVQWLRLGNPSSGAPPPS |
| 153 | YaPGTFTSDaaAAibLEDK*YQAibAFIAWLAibQGWPSSGAPPPS |
| 154 | YGaGTFTSDIRAYLDEK*WAKAFVAWLVsGMPSSGAPPPS |
| 155 | YGWGTFTSDPINAibLDLK*AibQKDFVNWLPMGAPSSGAPPPS |
| 156 | HsNGTFTSDsYaYLERK*DQKAFIEWLWSGPPSSGAPPPS |
| 157 | YsMGTFTSDQGKYLEAK*SAQAFINWLMAibGAPSSGAPPPS |
| 158 | YaFGTFTSDLTRAibLDAibK*TAKAFIEWLDaGMPSSGAPPPS |
| 159 | YAibKGTFTSDAibIYYLDWK*NAAibAFVNWLHIGIPSSGAPPPS |
| 160 | HAibQGTFTSDWsAibAibLEKK*VQAibEFVNWLAibTGDP |
| | SSGAPPPS |
| 161 | FssGTFTSDDRaAibLDSK*aAQAFVAWLAibAGTPSSGAPPPS |
| 162 | YaIGTFTSDMKIYLDEK*aAKAFVQWLANGSPSSGAPPPS |
| 163 | FGQGTFTSDYDQAibLDNK*PQAibDFIEWLMYGEPSSGAPPPS |
| 164 | FsMGTFTSDLDRAibLDWK*IQKDFVQWLVSGTPSSGAPPPS |
| 165 | YsVGTFTSDLHSYLDLK*GAQDFVNWLVWGQPSSGAPPPS |
| 166 | HAibIGTFTSDDVYYLDLK*PQKEFVEWLGLGSPSSGAPPPS |
| 167 | FGVGTFTSDVMIAibLDIK*EQKEFINWLQSGEPSSGAPPPS |
| 168 | HAibFGTFTSDHAibsYLEsK*MAQDFINWLIIGsPSSGAPPPS |
| 169 | HaGGTFTSDPAKYLDSK*VQQEFVAWLAFGAibPSSGAPPPS |
| 170 | YaNGTFTSDVsFAibLEYK*DQKDFIQWLASGQPSSGAPPPS |
| 171 | FsVGTFTSDNRQYLDVK*NQAibDFIQWLPWGIPSSGAPPPS |
| 172 | HAibVGTFTSDKETYLDIK*KAAibDFVEWLSTGRPSSGAPPPS |
| 173 | FGWGTFTSDDFNAibLEYK*HAAibEFVNWLDVGQPSSGAPPPS |
| 174 | FaYGTFTSDQSYAibLDYK*TAKDFIQWLTKGRPSSGAPPPS |
| 175 | FGPGTFTSDDAibIYLEaK*NAQAFIEWLFSGQPSSGAPPPS |
| 176 | YAibIGTFTSDSsIAibLDSK*AQAibDFIQWLLFGQPSSGAPPPS |
| 177 | HsGGTFTSDPYGAibLEQK*MAKEFVEWLMTGKPSSGAPPPS |
| 178 | HaEGTFTSDYAKYLDAK*RAKEFIAWLVNGGPSSGAPPPS |
| 179 | YAibQGTFTSDYSImeILDKK*AQAibAFIEYLLEGGPSSGAPPPS |
| 180 | HGQGTFTSDYSKYLDK*RRAQDFVEWLKNGGPSSGAPPPS |
| 181 | HAibQGTFTSDYSKAibLDK*RRAQDFVEWLKNGGPSSGAPPPS |
| 182 | HAibQGTFTSDYSKYLDK*RRAQDFVEWLKNGGPSSGAPPPS |
| 183 | X1X2X3GTFTSDX4X5X6X7LX8X9X10X11X12X13X14 |
| | FX15X16WLX17X18GX19PSSGAPPPSX20 |
| 184 | X1X2X3GTFTSDX4X5X6X7LX8X9X10X11X12X13X14 |
| | FX15X16WLX17X18GX19PSSGAPPPSX20X21 |
| 185 | HAibEGTFTSDVSSYLEGQAAKEFIAWLVKGRK* |
| | (Albenatidex-AB-014 when bound to |
| | CCys₃₄ in retro-Michael resistant/ |
| | stereopreferred or stereoselective |
| | manner via maleimide |
| | reactive group) |
| 186 | HAibEGTFTSDVSSYLEGQAAKEFIAWLVKGRK\hat{ } |
| 187 | HAibEGTFTSDVSSYLEGQAAKEFIAWLVKGRK″ |
| 188 | HAibEGTFTSDVSSYLEGQAAKEFIAWLVKGRK′ |
| 189 | HAibEGTFTSDVSSYLEGQAAKEFIAWLVKGR9K* |
| 190 | HGEGTFTSDLSKQMEEEAVRLFIEWLKNGGPSSGAPPPSK\hat{ } |
| 191 | HGEGTFTSDLSKQMEEEAVRLFIEWLKNGGPSSGAPPPSK″ |
| 192 | HGEGTFTSDLSKQMEEEAVRLFIEWLKNGGPSSGAPPPSK′ |
| 193 | HGEGTFTSDLSKQMEEEAVRLFIEWLKNGGPSSGAPPPS9K* |
| 194 | HsQGTFTSDYSKYLDSRRAQDFVQWLMNTGPSSGAPPPSK* |
| 195 | HsQGTFTSDYSKYLDSRRAQDFVQWLMNTGPSSGAPPPSK\hat{ } |
| 196 | HsQGTFTSDYSKYLDSRRAQDFVQWLMNTGPSSGAPPPSK″ |
| 197 | HsQGTFTSDYSKYLDSRRAQDFVQWLMNTGPSSGAPPPSK′ |
| 198 | HsQGTFTSDYSKYLDSRRAQDFVQWLMNTGPSSGAPPPS9K* |
| 199 | YaEGTFISDYSIAMDKIHQQDFVNWLLAQGPSSGAPPPSK* |
| 200 | YaEGTFISDYSIAMDKIHQQDFVNWLLAQGPSSGAPPPSK\hat{ } |
| 201 | YaEGTFISDYSIAMDKIHQQDFVNWLLAQGPSSGAPPPSK″ |
| 202 | YaEGTFISDYSIAMDKIHQQDFVNWLLAQGPSSGAPPPSK′ |
| 203 | YaEGTFISDYSIAMDKIHQQDFVNWLLAQGPSSGAPPPS9K* |
| 204 | HAEGTFTSDVSSYLEGQAAK*EFIAWLVRGR |
| 205 | HAibEGTFTSDVSSYLEGQAAK*EFIAWLVRGRG |
| 206 | HVEGTFTSDVSSYLEEQAAREFIK*WLVRGRG |
| 207 | HaEGTFTSDVSSYLEGQAAKEFIAWLVNGGPSSGAPPPSK\hat{ } |
| 208 | HaEGTFTSDVSSYLEGQAAKEFIAWLVNGGPSSGAPPPSK″ |
| 209 | HaEGTFTSDVSSYLEGQAAKEFIAWLVNGGPSSGAPPPSK′ |
| 210 | HaEGTFTSDVSSYLEGQAAKEFIAWLVNGGPSSGAPPPS9K* |
| 211 | HaEGTFTSDVASYLEGQAAKEFIAWLVNGGPSSGAPPPSK* |
| 212 | YAibEGTFTSDYSIYLDKQAAAibEFVNWLLAGGPSSGAPPPSK* |
| 213 | HAibQGTFTSDYSKYLDEKKAK*EFVEWLLEGGPSSG |
| 214 | HAibQGTFTSDYSKYLDEK*AAKEFIQWLLQT |
| 215 | HAibQGTFTSDYSKYLDEKRAKEFVQWLMNTC* |
| 216 | HsQGTFTSDK*SKYLDARAAQDFVQWLLDT |
| 217 | HAc4cQGTFTSDYSKYLDERAAKDFIK*WLESA |
| 218 | HsQGTFTSDLSKQK*ESKAAQDFIEWLKAGGPSSGAPPPS |
| 219 | HSQGTFTSDK*SEYLDSERARDFVAWLEAGG |
| 220 | HAibQGTFTSDK*SKYLDERAAQDFVQWLLDGGPSSGAPPPS |
| 221 | HSQGTFTSDK*SKYLDERRAQDFVQWLLDGGPSSGAPPPS |
| 222 | HAibHGTGTSDLSKLK*EEQRQAibEFIEWLKAaGPPSAibKPPPK |
| 223 | HaQGTFTSDYAKYLDARRAKEFIAWLVNGGPSSGAPPPSK* |
| | (Albutide AB-044 when |
| | bound to Cys₃₄ in retro-Michael |
| | resistant/stereopreferred or stereoselective |
| | manner via |
| | maleimide reactive group) |
| 224 | HaQGTFTSDYAKYLDARRAKEFIAWLVNGGPSSGAPPPSK\hat{ } |
| 225 | HaQGTFTSDYAKYLDARRAKEFIAWLVNGGPSSGAPPPSK″ |
| 226 | HaQGTFTSDYAKYLDARRAKEFIAWLVNGGPSSGAPPPSK′ |
| 227 | HaEGTFTSDYAKYLDARRAKEFIAWLVNGGPSSGAPPPSK\hat{ } |
| 228 | HaEGTFTSDYAKYLDARRAKEFIAWLVNGGPSSGAPPPSK″ |
| 229 | HaEGTFTSDYAKYLDARRAKEFIAWLVNGGPSSGAPPPSK′ |
| 230 | HaEGTFTSDYAKYLDARRAKEFIAWLVNGGPSSGAPPPS9K* |
| 231 | YAEGTFISDYSIAMDKIHQQDFVNWLLAQKGKKNDWKHNITQ |
| 232 | HaEGTFTSDVASYLEGQAAKEFIAWLVNGGPSSGAPPPS9K* |
| 233 | HAibEGTFTSDVSSYLEGQAAK\hat{ }EFIAWLVRGRG |
| 234 | HAibEGTFTSDVSSYLEGQAAKEFIAWLVRGRGK* |
| 235 | HAibEGTFTSDVSSYLEGQAAAibEFIAWLVRGRGK″ |
| 236 | YAibEGTFTSDYSIAibLDKIAQK″AFVQWLIAGGPSSGAPPPS |
| 237 | YAibEGTFTSDYSIAibLDKIAQK\hat{ }AFVQWLIAGGPSSGAPPPS |
| 238 | YAibEGTFTSDYSIYLDKQAAAibEFVQWLLAGGPSSGAPPPSK\hat{ } |
| 239 | YAibEGTFTSDYSIYLDKQAAAibEFVQWLLAGGPSSGAPPPSK# |
| 240 | HAibQGTFTSDYSKYLDEKKAK\hat{ }EFVEWLLEGGPSSG |
| 241 | HAibQGTFTSDYSKYLDEKKAK″EFVEWLLEGGPSSG |
| 242 | HAibQGTFTSDYSKYLDEKKAK#EFVEWLLEGGPSSG |
| 243 | HAibQGTFTSDYSKYLDEKKAKEFVEWLLEGGPSSGK* |
| 244 | HAibQGTFTSDYSKYLDEK\hat{ }AAKEFIQWLLQT |
| 245 | HAibQGTFTSDYSKYLDEK″AAKEFIQWLLQT |
| 246 | HsQGTFTSDLSKOK\hat{ }ESKAAQDFIEWLKAGGPSSGAPPPS |
| 247 | HsQGTFTSDLSKQK#ESKAAQDFIEWLKAGGPSSGAPPPS |
| 248 | HsQGTFTSDLSKQLESKAAQDFIEWLKAGGPSSGAPPPSK* |
| 249 | HsQGTFTSDLSKQLESKAAQDFIEWLKAGGPSSGAPPPSK″ |
| 250 | HSQGTFTSDLSEYLDSERARDFVAWLEAGGK** |
| 251 | YAibQGTFTSDYSIMeLLDKK\hat{ }AQAibAFIEYLLEGGPSSGAPPPS |
| 252 | YAibQGTFTSDYSIMeLLDKK″AQAibAFIEYLLEGGPSSGAPPPS |
| 253 | YAibQGTFTSDYSIMeLLDKK#AQAibAFIEYLLEGGPSSGAPPPS |
| 254 | YAibQGTFTSDYSIMeLLDKIAQAibAFIEYLLEGGPSSGAPPPSK\hat{ } |
| 255 | YAibQGTFTSDYSIMeLLDKIAQAibAFIEYLLEGGPSSGAPPPSK″ |
| 256 | YAibQGTFTSDYSIMeLLDKIAQAibAFIEYLLEGGPSSGAPPPSK# |
| 257 | HSQGTFTSDK\hat{ }SKYLDERRAQDFVQWLLDGGPSSGAPPPS |
| 258 | HSQGTFTSDK″SKYLDERRAQDFVQWLLDGGPSSGAPPPS |
| 259 | HSQGTFTSDYSKYLDERRAQDFVQWLLDGGPSSGAPPPSK* |
| 260 | HSQGTFTSDYSKYLDERRAQDFVQWLLDGGPSSGAPPPSK** |
| 261 | HAibHGTGTSDLSKLK\hat{ }EEQRQAibEFIEWLKAaGPPSAibKPPPK |
| 262 | HAibHGTGTSDLSKLLEEQRQAibEFIEWLKAaGPPSAibKPPPK* |
| 263 | HAibHGTGTSDLSKLLEEQRQAibEFIEWLKAaGPPSAibKPPP9K* |
| 264 | YaEGTFTSDYAKYLDARRAAibEFIAWLVNGGPSSGAPPPSK* |
| 265 | YAibEGTFTSDYAKYLDARRAAibEFIAWLVNGGPSSGAPPPSK* |
| 266 | YAibEGTFTSDYSIAibLDKRRAAibEFIAWLVNGGPSSGAPPPSK* |
| 267 | HaEGTFTSDYSIAibLDKRRAAibEFIAWLVNGGPSSGAPPPSK* |
| 268 | YaEGTFTSDYSIAibLDKRRAAibEFIAWLVNGGPSSGAPPPSK* |
| 269 | YAibEGTFTSDYSIAibLDKIAQAibAFIAWLVNGGPSSGAPPPSK* |
| 270 | HaEGTFTSDYSIAibLDKIAQAibAFIAWLVNGGPSSGAPPPSK* |
| 271 | YaEGTFTSDYSIAibLDKIAQAibAFIAWLVNGGPSSGAPPPSK* |
| 272 | HaEGTFTSDYSIAibLDKIAQAibAFVQWLIAGGPSSGAPPPSK* |
| 273 | YaEGTFTSDYSIAibLDKIAQAibAFVQWLIAGGPSSGAPPPSK* |
| 274 | YAibEGTFTSDYSIAibLDKIAQAibAFVQWLIAGGPSSGAPPPS9K* |
| 275 | HaEGTFTSDYAKYLDARRAAibEFVQWLIAGGPSSGAPPPSK* |
| 276 | YaEGTFTSDYAKYLDARRAAibEFVQWLIAGGPSSGAPPPSK* |
| 277 | YAibEGTFTSDYAKYLDARRAAibEFVQWLIAGGPSSGAPPPSK* |
| 278 | HaEGTFTSDYAKYLDKIAQAibAFVQWLIAGGPSSGAPPPSK* |
| 279 | YaEGTFTSDYAKYLDKIAQAibAFVQWLIAGGPSSGAPPPSK* |
| 280 | YAibEGTFTSDYAKYLDKIAQAibAFVQWLIAGGPSSGAPPPSK* |
| 281 | YAibQGTFTSDYAKYLDARRAKEFIAWLVNGGPSSGAPPPSK* |
| 282 | YaQGTFTSDYAKYLDARRAKEFIAWLVNGGPSSGAPPPSK* |
| 283 | YAibQGTFTSDYSIMeLLDKRRAKEFIAWLVNGGPSSGAPPPSK* |
| 284 | YaQGTFTSDYSIMeLLDKRRAKEFIAWLVNGGPSSGAPPPSK* |
| 285 | HaQGTFTSDYSIMeLLDKRRAKEFIAWLVNGGPSSGAPPPSK* |
| 286 | YAibQGTFTSDYSIMeLLDKIAQAibAFIAWLVNGGPSSGAPPPSK* |
| 287 | YaQGTFTSDYSIMeLLDKIAQAibAFIAWLVNGGPSSGAPPPSK* |
| 288 | HaQGTFTSDYSIMeLLDKIAQAibAFIAWLVNGGPSSGAPPPSK* |
| 289 | YaQGTFTSDYSIMeLLDKIAQAibAFIEYLLEGGPSSGAPPPSK* |
| 290 | HaQGTFTSDYSIMeLLDKIAQAibAFIEYLLEGGPSSGAPPPSK* |
| 291 | YAibQGTFTSDYSIMeLLDKIAQAibAFIEYLLEGGPSSGAPPPS9K* |
| 292 | HaQGTFTSDYAKYLDARRAKEFIEYLLEGGPSSGAPPPSK* |
| 293 | HAibQGTFTSDYAKYLDARRAKEFIEYLLEGGPSSGAPPPSK* |
| 294 | YAibQGTFTSDYAKYLDARRAKEFIEYLLEGGPSSGAPPPSK* |
| 295 | YaQGTFTSDYAKYLDARRAKEFIEYLLEGGPSSGAPPPSK* |
| 296 | HaQGTFTSDYAKYLDKIAQAibAFIEYLLEGGPSSGAPPPSK* |
| 297 | HAibQGTFTSDYAKYLDKIAQAibAFIEYLLEGGPSSGAPPPSK* |
| 298 | YAibQGTFTSDYAKYLDKIAQAibAFIEYLLEGGPSSGAPPPSK* |
| 299 | YaQGTFTSDYAKYLDKIAQAibAFIEYLLEGGPSSGAPPPSK* |
| 300 | HAibQGTFTSDYSIMeLLDKIAQAibAFIEYLLEGGPSSGAPPPSK* |
| 301 | HaQGTFTSDYAKYLDARRAKEFIAWLVNGGPSSGAPPPSK# |
| 302 | HaEGTFTSDYAKYLDAK*RAKEFIAWLVNGGPSSGAPPPS |
| 303 | HaEGTFTSDYAKYLDAKARAKEFIAWLVNGGPSSGAPPPS |
| 304 | HaEGTFTSDYAKYLDAK&RAKEFIAWLVNGGPSSGAPPPS |
| 305 | HaEGTFTSDYAKYLDAK′RAKEFIAWLVNGGPSSGAPPPS |
| 306 | HaEGTFTSDYAKYLDARRAK\hat{ }EFIAWLVNGGPSSGAPPPS |
| 307 | HaEGTFTSDYAKYLDARRAK&EFIAWLVNGGPSSGAPPPS |
| 308 | HaEGTFTSDYAKYLDARRAK′EFIAWLVNGGPSSGAPPPS |
| 309 | HaQGTFTSDYAKYLDARRAKEFIAWLVNGGPSSGAPPPSK2 |
| 310 | HaQGTFTSDYAKYLDARRAKEFIAWLVNGGPSSGAPPPSK3 |
| 311 | HaQGTFTSDYAKYLDARRAKEFIAWLVNGGPSSGAPPPSK1 |
| 312 | HaQGTFTSDYAKYLDARRAKEFIAWLVNGGPSSGAPPPSC2 |
| 313 | HaQGTFTSDYAKYLDAK2RAKEFIAWLVNGGPSSGAPPPS |
| 314 | HaQGTFTSDYAKYLDAK5RAKEFIAWLVNGGPSSGAPPPS |
| 315 | HaQGTFTSDYAKYLDAKIRAKEFIAWLVNGGPSSGAPPPS |
| 316 | HaQGTFTSDYAKYLDAK4RAKEFIAWLVNGGPSSGAPPPS |
| 317 | YAibQGTFTSDYSILLDKK*AQAibAFIEYLLEGGPSSGAPPPS |
| 318 | HADGSFSDEMNTILDNLAARDFINWLIQTKITD |
| 319 | HSDAVFTDNYTRLRKQMAVKKYLNSILN |
| 320 | KCNTATCATQRLANFLVHSSNNFGAILSSTNVGSNTY |
| 321 | YADAIFTNSYRKVLGQLSARKLLQDIMSR |
| 322 | X1X2X3GTFTSDX4X5X6X7LX8X9X10X11X12 |
| | X13X14FX15X16WLX17X18GX19PSSG |
| | APPPSX20 |
| 323 | X1X2X3GTFTSDX4X5X6X7LX8X9X10X11X12 |
| | X13X14FX15X16WLX17X18GX19PSSG |
| | APPPS |
| 324 | X19PSSGAPPPSX20 |
| 325 | X11X12X13X14FX15X16WLX17X18G |
| 326 | X1X2X3GTFTSDX4X5X6X7LX8X9X10 |
| 327 | X19PSSGAPPPSK |
| 328 | X11X12X13X14FX15X16WLX17X18G- |
| | X19PSSGAPPPSX20 |
| 329 | X1X2X3GTFTSDX4X5X6X7LX8X9X10- |
| | X11X12X13X14FX15X16WLX17X18G- |
| | X19PSSGAPPPSX20 |
| 330 | HaEGTFTSDVSSYLEGQAAKEFIAWLVKGRPSSGAPPPSK* |
| 331 | HAEGTFTSDVSSYLEGQAAKEFIAWLVKGRPSSGAPPPSK* |
| 332 | HaEGTFTSDVSSYLEGQAAKEFIAWLVNGGK* |
| 333 | HaEGTFTSDINKVLDTIAAKEFIAWLVKGRK* |
| 334 | HaEGTFTSDAEKAKEAEKAKEFIAWLVKGRK* |
| 335 | HaEGTFTSDAibEKAibKEAibEKAKEFIAWLVKGRK* |
| 336 | HaEGTFTSDAibESAibKEAibEKAibKEFIAWLVKGRP |
| | SSGAPPPSK* |
| 337 | HaEGTFTSDVAibSYAibEGQAibAKEFIAWLVKGRPSSGAPPPSK* |
| 338 | HaEGTFTSDVSSYLEGK*AAKEFIAWLVKGRPSSGAPPPS |
| 339 | HaEGTFTSDVSSYLEGQAAKEFIAWLVKGRLLLLLLLLLK* |
| 340 | HaEGTFTSDVSSYLEGQAAKEFIAWLVKGRPEAPTDPEAPTD |
| 341 | HGEGTFTSDLSKQMEEEAVRLFIEWLKNGGGGGGGGGGGK* |
| 342 | HaEGTFTSDVSSYLEGQAAKEFIAWLVKGRK% |
| 343 | HaEGTFTSDVSSYLEGK*AAKEFIAWLVKGR |
| 344 | HaEGTFTSDVSSYLEGQAAKEFIAWLVKGRK@ |
| 345 | HSQGTFTSDYSKYLDSRRAQDFVQWLMNTKRNKNNIA |
| 346 | HaEGTFTSDVSSYLEGQAAKEFIAWLVKGPEAPTDPEAPTD |
| 347 | HaEGTFTSDVSSYLEGQAAKEFIAWLVKGRK** |
| 348 | HaEGTFTSDINKVLDIIAAKEFIAWLVKGRPSSGAPPPSK* |
| 349 | HaEGTFTSDINKVLDIIAAKEFIAWLVKGRK* |
| 350 | HGEGTFTSDLSKQMEEEAVRLFIEWLKNGGPSSGAPPPSK** |
| 351 | HAibEGTFTSDYSKYLDKIRAQEFVAWLMNGGPSSGAPPPSK* |
| 352 | HAibQGTFTSDYSKYLDKIAAQDFVAWLLNGGPSSGAPPPSK* |
| 353 | HAibQGTFTSDYAKYLDKIAAQDFVAWLLDGGPSSGAPPPSK* |
| 354 | HAibQGTFTSDYSKYLDKIAAQDFVAWLLDGGPSSGAPPPSK* |
| 355 | HAibQGTFTSDYSKYLDKIAAQDFVAYLLDGGPSSGAPPPSK* |
| 356 | HAibQGTFTSDLSKYLDEIAVQDFIEWLLDGGPSSGAPPPSK* |
| 357 | HSQGTFTSDYSKYLDSRRAQDFVQWLMNTKRNRNNIA |
| 358 | IKPEAPGEDASPEELNRYYASLRHYLNLVTRQRY |
| 359 | HAibEGTFTSDVSSYLEGQALRHYINWLTRQRY |
| 360 | HGEGTFTSDLSKQMEEEAVRLFIEWLKNGGPSSRHYLNLVTRQRY |
| 361 | HdsQGTFTSDLSKYLEEEAVREFIAWLKNGGPSSGAPPPSRHYLN |
| | LVTRQRY |
| 362 | HdsQGTYTNDVSKYXDSRRAQDFIEWLKNGGPSSGAPPPS |
| 363 | HdsQGTYTNDVSKYKDSRRAQDFIEWLKNGGPSSGAPPPSC |
| 364 | HdsQGTFTSDLSKQKDSRRAQDFIEWLKNGGPSSGAPPPSC |
| 365 | HdsQGTYTNDVSKYXDSRRAQDFIEWLKNGGPSSGAPPPSC |
| 366 | PKPEAPGKDASPEEWNRYYADLRHYLNWLTRQRY |
| 367 | IKPEAPGEDASPEELNRYYASLRHYLNWVTRQXY |
| 368 | YAibEGTFTSDYSIYLDKQAAAibAFVQWLIAGGPSSGAPPPSK* |
| 369 | YAibEGTFTSDYSIYLDKQAAAibAFVQWLLAGGPSSGAPPPSK* |
| 370 | YAibEGTFTSDYSIYLDKQAAAibEFVQWLIAGGPSSGAPPPSK* |
| 371 | HaEGTFTSDYSIYLDKQAAAibEFVQWLLAGGPSSGAPPPSK* |
| 372 | YaEGTFTSDYSIYLDKQAAAibEFVQWLLAGGPSSGAPPPSK* |
| 373 | HAibEGTFTSDYSIYLDKQAAAibEFVQWLLAGGPSSGAPPPSK* |
| 374 | HaEGTFTSDYSIYLDKQAAAibAFVQWLIAGGPSSGAPPPSK* |
| 375 | HaEGTFTSDYSIYLDKQAAAibAFVQWLLAGGPSSGAPPPSK* |
| 376 | HaEGTFTSDYSIYLDKQAAAibEFVQWLIAGGPSSGAPPPSK* |
| 377 | YaEGTFTSDYSIYLDKQAAAibAFVQWLIAGGPSSGAPPPSK* |
| 378 | YaEGTFTSDYSIYLDKQAAAibAFVQWLLAGGPSSGAPPPSK* |
| 379 | YaEGTFTSDYSIYLDKQAAAibEFVQWLIAGGPSSGAPPPSK* |
| 380 | YAibEGTFTSDYAKYLDARRAKEFVQWLLAGGPSSGAPPPSK* |
| 381 | YAibEGTFTSDYSKYLDARRAKEFVQWLLAGGPSSGAPPPSK* |
| 382 | YAibEGTFTSDYSKYLDKRRAKEFVQWLLAGGPSSGAPPPSK* |
| 383 | HaEGTFTSDYAKYLDARRAKEFVQWLLAGGPSSGAPPPSK* |
| 384 | HaEGTFTSDYSKYLDARRAKEFVQWLLAGGPSSGAPPPSK* |
| 385 | HaEGTFTSDYSKYLDKRRAKEFVQWLLAGGPSSGAPPPSK* |
| 386 | YaEGTFTSDYAKYLDARRAKEFVQWLLAGGPSSGAPPPSK* |
| 387 | YaEGTFTSDYSKYLDARRAKEFVQWLLAGGPSSGAPPPSK* |
| 388 | YaEGTFTSDYSKYLDKRRAKEFVQWLLAGGPSSGAPPPSK* |
| 389 | HAibEGTFTSDYAKYLDARRAKEFVQWLLAGGPSSGAPPPSK* |
| 390 | HAibEGTFTSDYSKYLDARRAKEFVQWLLAGGPSSGAPPPSK* |
| 391 | HAibEGTFTSDYSKYLDKRRAKEFVQWLLAGGPSSGAPPPSK* |
| 392 | YAibEGTFTSDYAKYLDARRAKAFVQWLIAGGPSSGAPPPSK* |
| 393 | YAibEGTFTSDYSKYLDARRAKAFVQWLIAGGPSSGAPPPSK* |
| 394 | YAibEGTFTSDYSKYLDKRRAKAFVQWLIAGGPSSGAPPPSK* |
| 395 | HaEGTFTSDYAKYLDARRAKAFVQWLIAGGPSSGAPPPSK* |
| 396 | HaEGTFTSDYSKYLDARRAKAFVQWLIAGGPSSGAPPPSK* |
| 397 | HaEGTFTSDYSKYLDKRRAKAFVQWLIAGGPSSGAPPPSK* |
| 398 | YaEGTFTSDYAKYLDARRAKAFVQWLIAGGPSSGAPPPSK* |
| 399 | YaEGTFTSDYSKYLDARRAKAFVQWLIAGGPSSGAPPPSK* |
| 400 | YaEGTFTSDYSKYLDKRRAKAFVQWLIAGGPSSGAPPPSK* |
| 401 | HAibEGTFTSDYAKYLDARRAKAFVQWLIAGGPSSGAPPPSK* |
| 402 | HAibEGTFTSDYSKYLDARRAKAFVQWLIAGGPSSGAPPPSK* |
| 403 | HAibEGTFTSDYSKYLDKRRAKAFVQWLIAGGPSSGAPPPSK* |
| 404 | HaQGTFTSDLSKQLESKAAQDFIEWLKAGGPSSGAPPPSK* |
| 405 | HsQGTFTSDYAKYLDARRAKEFIEWLKAGGPSSGAPPPSK* |
| 406 | HsQGTFTSDYSKYLDARRAKEFIEWLKAGGPSSGAPPPSK* |
| 407 | HsQGTFTSDYAKYLDSRRAKEFIEWLKAGGPSSGAPPPSK* |
| 408 | HsQGTFTSDYSKYLDSRRAKEFIEWLKAGGPSSGAPPPSK* |
| 409 | HaQGTFTSDYSKYLDSRRAKEFIEWLKAGGPSSGAPPPSK* |
| 410 | HaQGTFTSDYAKYLDARRAKEFIEWLKAGGPSSGAPPPSK* |
| 411 | HaQGTFTSDLSKQLESKAAQDFIAWLVNGGPSSGAPPPSK* |
| 412 | HaQGTFTSDYAKYLDARRAKDFIEWLKAGGPSSGAPPPSK* |
| 413 | HaQGTFTSDYSKYLDARRAQEFIEWLKAGGPSSGAPPPSK* |
| 414 | HaQGTFTSDYAKYLDSRAAKEFIEWLKAGGPSSGAPPPSK* |
| 415 | HaQGTFTSDYSKYLDSKRAKEFIEWLKAGGPSSGAPPPSK* |
| 416 | HaQGTFTSDYAKYLEARRAKEFIEWLKAGGPSSGAPPPSK* |
| 417 | HaQGTFTSDYAKQLEARRAKEFIEWLKAGGPSSGAPPPSK* |
| 418 | HAibQGTFTSDYSKYLDERAAQAFIEYLLEGGPSSGAPPPSK* |
| 419 | HaQGTFTSDYSKYLDERAAQDFVQWLLDGGPSSGAPPPSK* |
| 420 | HaQGTFTSDYSKYLDERAAQAFIEYLLEGGPSSGAPPPSK* |
| 421 | HAibQGTFTSDYSKYLDARRAKEFVQWLLDGGPSSGAPPPSK* |
| 422 | HAibQGTFTSDYSKYLDARRAKEFIEYLLEGGPSSGAPPPSK* |
| 423 | HAibQGTFTSDYAKYLDARRAKEFVQWLLDGGPSSGAPPPSK* |
| 424 | HaQGTFTSDYSKYLDARRAKEFVQWLLDGGPSSGAPPPSK* |
| 425 | HaQGTFTSDYAKYLDARRAKEFVQWLLDGGPSSGAPPPSK* |
| 426 | HaQGTFTSDYSKYLDARRAKEFIEYLLEGGPSSGAPPPSK* |
| 427 | HAibQGTFTSDYSKYLDERAAQAFIEYLLDGGPSSGAPPPSK* |
| 428 | HaQGTFTSDYSKYLDERAAQDFVQWLLEGGPSSGAPPPSK* |
| 429 | HaQGTFTSDYSKYLDERAAQAFIEYLLDGGPSSGAPPPSK* |
| 430 | HAibQGTFTSDYSKYLDARRAKEFVQWLLEGGPSSGAPPPSK* |
| 431 | HAibQGTFTSDYSKYLDARRAKEFIEYLLDGGPSSGAPPPSK* |
| 432 | HAibQGTFTSDYAKYLDARRAKEFVQWLLEGGPSSGAPPPSK* |
| 433 | HAibQGTFTSDYAKYLDARRAKEFIEYLLDGGPSSGAPPPSK* |
| 434 | HaQGTFTSDYSKYLDARRAKEFVQWLLEGGPSSGAPPPSK* |
| 435 | HaQGTFTSDYAKYLDARRAKEFVQWLLEGGPSSGAPPPSK* |
| 436 | HaQGTFTSDYSKYLDARRAKEFIEYLLDGGPSSGAPPPSK* |
| 437 | HaQGTFTSDYAKYLDARRAKEFIEYLLDGGPSSGAPPPSK* |
| 438 | HAibQGTFTSDYSKYLDERAAQAFIEWLLEGGPSSGAPPPSK* |
| 439 | HaQGTFTSDYSKYLDERAAQDFVQYLLDGGPSSGAPPPSK* |
| 440 | HaQGTFTSDYSKYLDERAAQAFIEWLLEGGPSSGAPPPSK* |
| 441 | HAibQGTFTSDYSKYLDARRAKEFVQYLLDGGPSSGAPPPSK* |
| 442 | HAibQGTFTSDYSKYLDARRAKEFIEWLLEGGPSSGAPPPSK* |
| 443 | HAibQGTFTSDYAKYLDARRAKEFVQYLLDGGPSSGAPPPSK* |
| 444 | HAibQGTFTSDYAKYLDARRAKEFIEWLLEGGPSSGAPPPSK* |
| 445 | HaQGTFTSDYSKYLDARRAKEFVQYLLDGGPSSGAPPPSK* |
| 446 | HaQGTFTSDYAKYLDARRAKEFVQYLLDGGPSSGAPPPSK* |
| 447 | HaQGTFTSDYSKYLDARRAKEFIEWLLEGGPSSGAPPPSK* |
| 448 | HaQGTFTSDYAKYLDARRAKEFIEWLLEGGPSSGAPPPSK* |
| 449 | HAibQGTFTSDYSKYLDERAAQAFVQYLLEGGPSSGAPPPSK* |
| 450 | HaQGTFTSDYSKYLDERAAQDFIEWLLDGGPSSGAPPPSK* |
| 451 | HaQGTFTSDYSKYLDERAAQAFVQYLLEGGPSSGAPPPSK* |
| 452 | HAibQGTFTSDYSKYLDARRAKEFIEWLLDGGPSSGAPPPSK* |
| 453 | HAibQGTFTSDYSKYLDARRAKEFVQYLLEGGPSSGAPPPSK* |
| 454 | HAibQGTFTSDYAKYLDARRAKEFIEWLLDGGPSSGAPPPSK* |
| 455 | HAibQGTFTSDYAKYLDARRAKEFVQYLLEGGPSSGAPPPSK* |
| 456 | HaQGTFTSDYSKYLDARRAKEFIEWLLDGGPSSGAPPPSK* |
| 457 | HaQGTFTSDYAKYLDARRAKEFIEWLLDGGPSSGAPPPSK* |
| 458 | HaQGTFTSDYSKYLDARRAKEFVQYLLEGGPSSGAPPPSK* |
| 459 | HaQGTFTSDYAKYLDARRAKEFVQYLLEGGPSSGAPPPSK* |
| 460 | HaEGTFTSDYAKYLDARRAKEFVQWLVNGGPSSGAPPPSK* |
| 461 | HaEGTFTSDYAKYLDARRAKEFIAWLLDGGPSSGAPPPSK* |
| 462 | HaEGTFTSDYAKYLDARRAKEFIEYLVNGGPSSGAPPPSK* |
| 463 | HaEGTFTSDYAKYLDARRAKEFIAYLLEGGPSSGAPPPSK* |
| 464 | HaEGTFTSDYAKYLDARRAKEFIEWLVNGGPSSGAPPPSK* |
| 465 | HaEGTFTSDYAKYLDARRAKEFVQWLKAGGPSSGAPPPSK* |
| 466 | YaEGTFTSDYAIYLDAQAQQDFVQWLLAGGPSSGAPPPSK\hat{ } |
| 467 | YaEGTFTSDYSIYLDKIAQQDFVQWLLAGGPSSGAPPPSK\hat{ } |
| 468 | HAibQGTFTSDYSKYLDERAAQDFVQWLLDGGPSSGAPPPSK* |
| 469 | HAibQGTFTSDYSKYLDKIAAQDFVAYLLDGGPSSGAPPPSK\hat{ } |
| 470 | YaEGTFTSDYSIYLDKIAQQDFVQWLLAGGPSSGAPPPSK* |
| 471 | YAibEGTFTSDYSIAibLDKIAQK\hat{ }AFV |
| | QWLIAGGPSSGAPPPS |

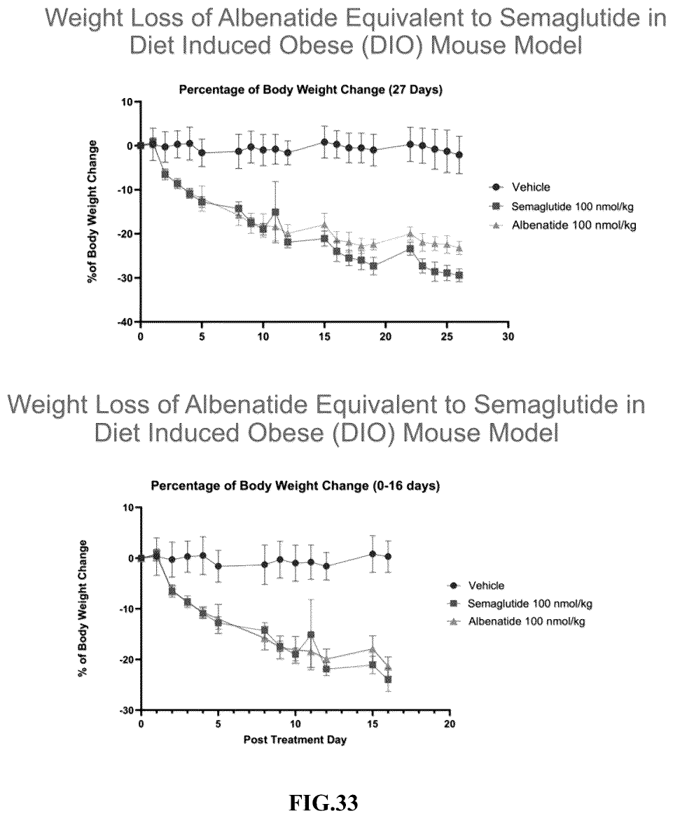

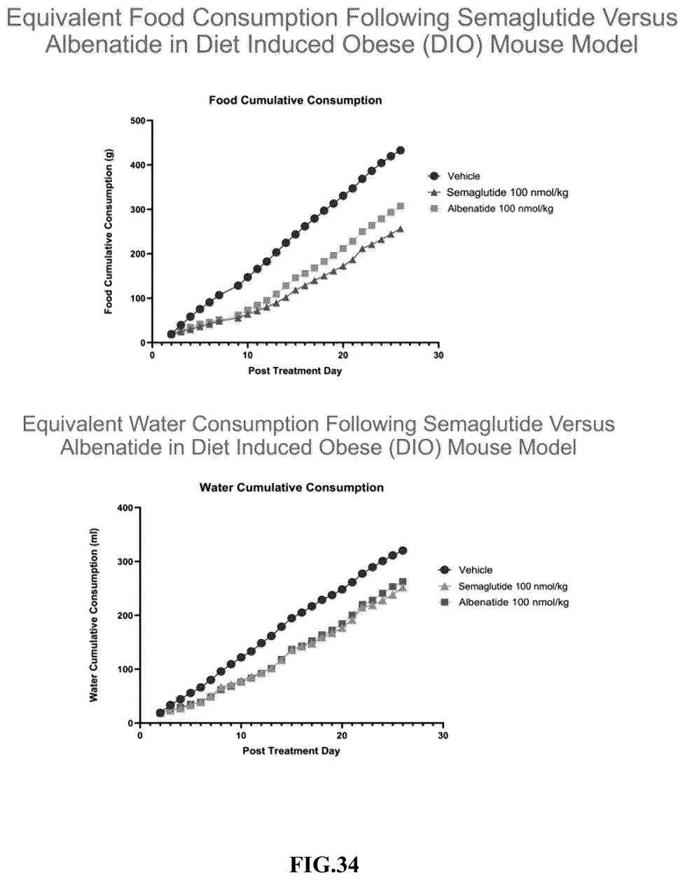

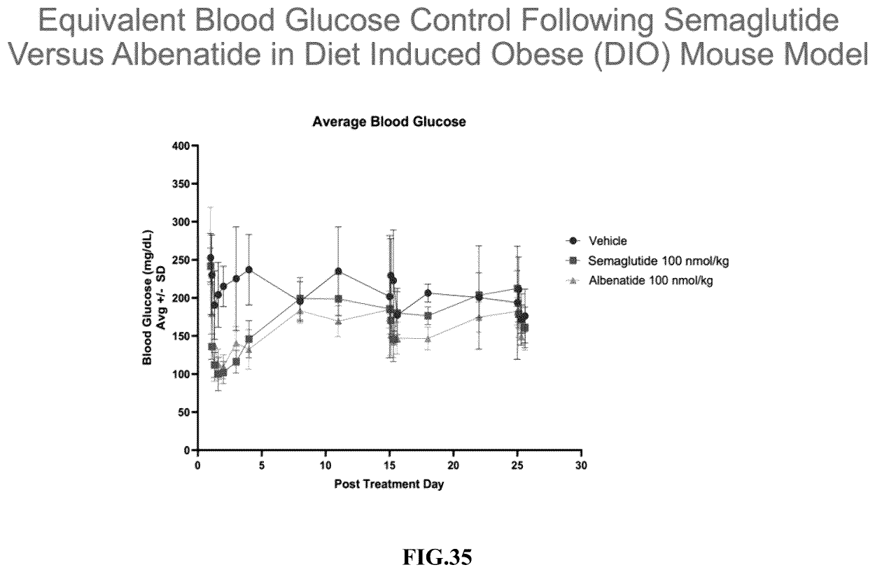

In further aspects of the inventive subject matter, it should be appreciated that the compositions and compounds presented herein will have significant biological activity in vivo when administered to a subject. For example, when retro-Michael resistant albenatide (AB-013-AEEA-succinimide (SEQ ID NO:2)-albumin) is administered to mice in a diet induced obese mouse model at a single dosage of 100 nmol/kg (s.c. injection), the retro-Michael resistant albenatide had significant physiological activity with regard to weight loss and was comparable to semaglutide over a period of 16 and 26 days as is shown in FIG. 33, which is also reflected in reduced cumulative food and water consumption show in FIG. 34. In addition, it was also observed in this model that the retro-Michael resistant albenatide had a profound and sustained effect on blood glucose levels as is exemplarily illustrated in FIG. 35.

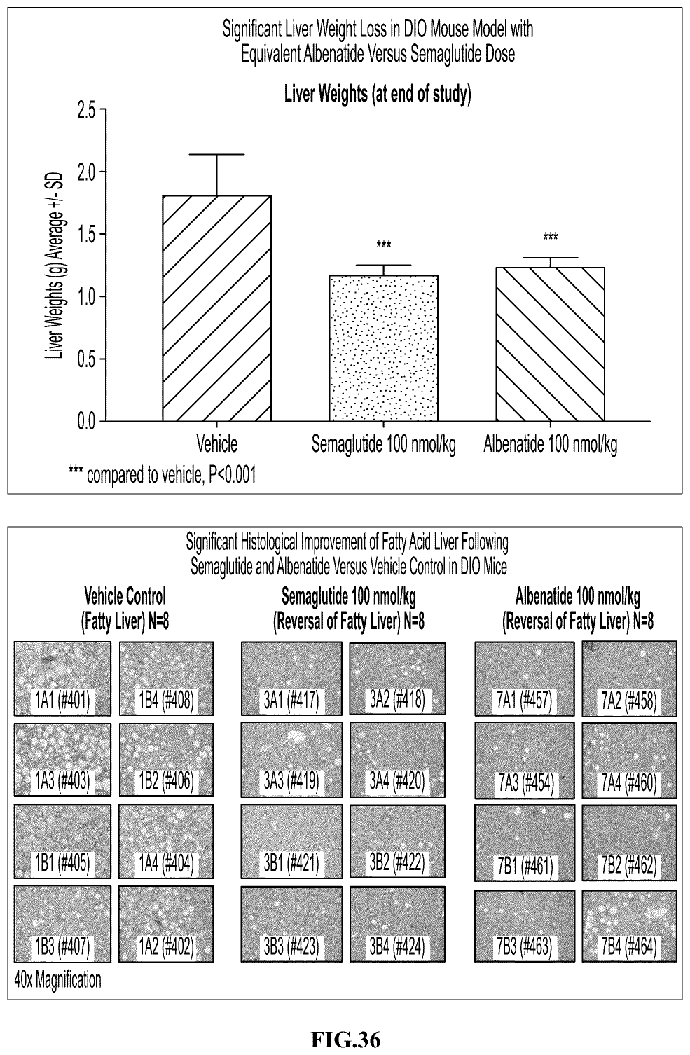

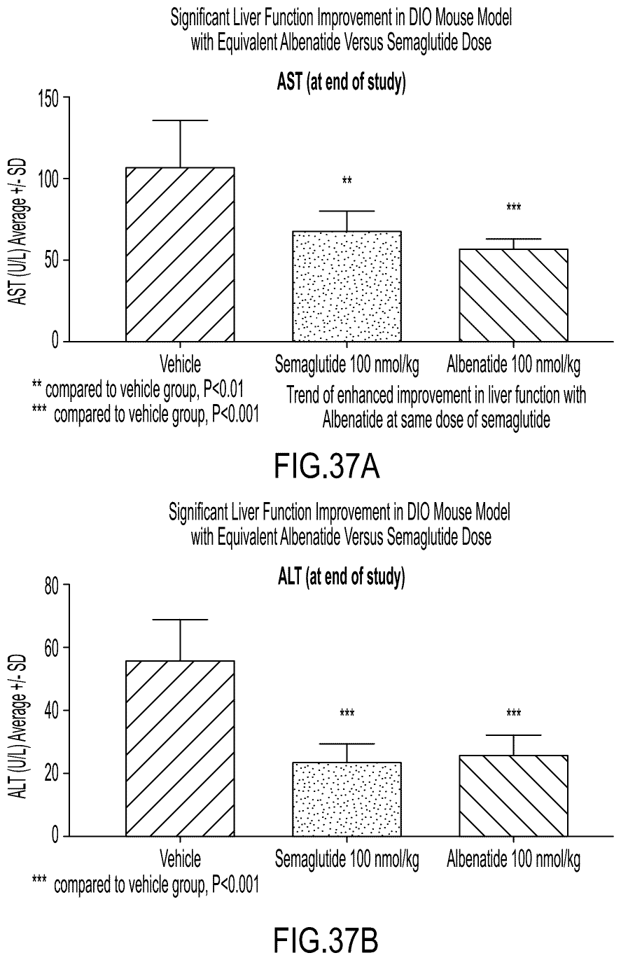

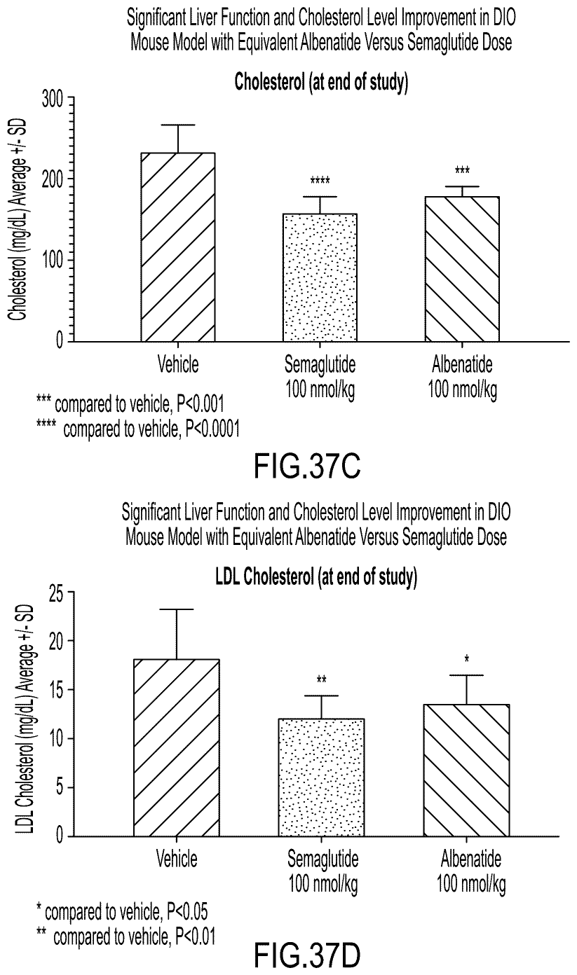

Moreover, and in addition to the metabolic modulation shown above, retro-Michael resistant albenatide also had a significant effect on hepatic lipid metabolism as can be readily taken from FIG. 36. Here, the single administration of retro-Michael resistant albenatide led to a substantial reduction in liver weight that was equivalent to the reduction obtained with semaglutide. This weight reduction was attributable to the quantity of lipid vesicles in the parenchyma as can be seen in the photomicrographs in FIG. 36. Notably, the retro-Michael resistant albenatide was thus effective in body weight control as well as with a reduction in blood glucose and was even capable of reversing fatty liver. In line with these observations were also the data shown in FIG. 37A and FIG. 37B that demonstrated a significant reduction in AST (37A) and ALT (37B) in serum. Likewise, administration of retro-Michael resistant albenatide further led to significantly reduced total serum cholesterol and LDL cholesterol as is shown in the data of FIG. 37C and FIG. 37D, respectively.

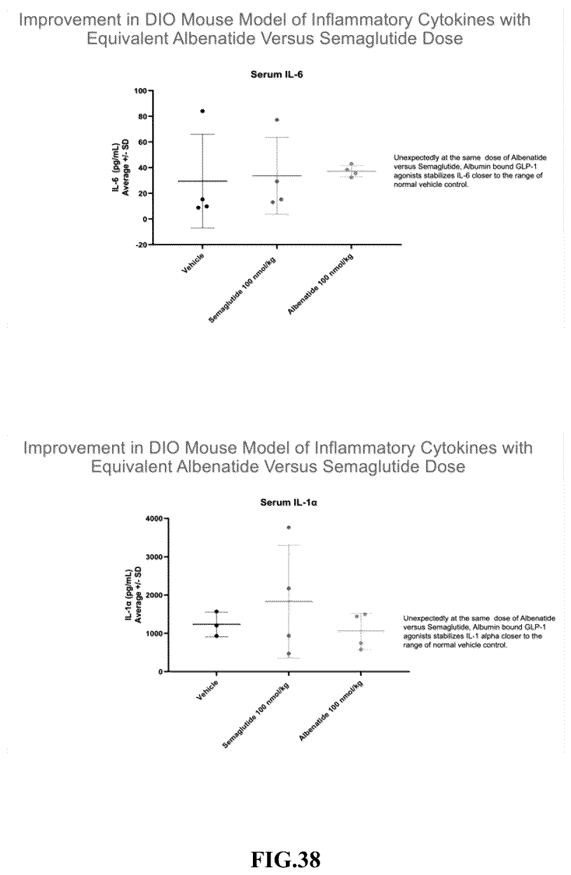

Additionally, the inventors also observed that retro-Michael resistant albenatide was able to reduce levels of pro-inflammatory cytokines IL-1a and IL-6 as depicted in FIG. 38. In this context, it should be particularly appreciated that the reduction and more compact data range is notable as the cytokines were measured at the end of the study, suggestion an even stronger effect closer to the time of administration.

Further aspects, examples, and methods of use for compounds presented herein are described in Applicant's International application with the title “Albumin Bound Macromolecule Tri-Agonist Activating GLP-1/GIP/Glucagon Receptors And Methods Therefor”, filed on or about Jun. 25, 2024, incorporated by reference herein.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No: 1-471 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:-(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants. Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance. Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, suitable calculations of the percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:-Sequence Identity=(N/T)*100. Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridizes to filter-bound DNA or RNA in 3× sodium chloride/sodium citrate (SSC) at approximately 45° C. followed by at least one wash in 0.2×SSC/0.1% SDS at approximately 20-65° C.

Alternatively, a substantially similar polypeptide may differ by at least 1, but less than 5, 10, 20, 50 or 100 amino acids from the sequences shown in, for example, in the amino acid sequence that are included within SEQ ID Nos: 1-471. Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

It should be noted that any language directed to a computer should be read to include any suitable combination of computing devices, including servers, interfaces, systems, databases, agents, peers, engines, modules, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method of producing a retro-Michael resistant Class B G protein-coupled receptor agonist (GPCR) fusion protein, comprising:
providing or producing a conformationally modified albumin;
covalently coupling a GPCR agonist peptide to Cys₃₄ of the conformationally modified albumin via a Michael addition reaction;
wherein the Michael addition reaction produces a stereopreferred or stereoselective chiral carbon atom in the Michael acceptor group; and
wherein the step of coupling is performed at a pH of pH<7.0.

2. The method of claim 1, wherein the conformationally modified albumin is modified by at least partial defatting.

3. The method of claim 1, wherein the pH is between 4.0 and 6.0.

4. The method of claim 1, wherein the GPCR agonist peptide comprises a linker with a planar Michael acceptor group.

5. The method of claim 4, wherein the planar Michael acceptor group comprises a maleimide group or a bromo maleimide group.

6. The method of claim 4, wherein the Michael addition reaction produces a stereopreferred chiral carbon atom in the Michael acceptor group.

7. The method of claim 4, wherein the Michael addition reaction produces a stereoselective chiral carbon atom in the Michael acceptor group.

8. The method of claim 1, wherein the GPCR agonist peptide has a structure according to any one of SEQ ID NO:1-471.

9. A Class B G protein-coupled receptor (GPCR) agonist fusion protein, comprising:
a conformationally modified albumin comprising a Cys₃₄ amino acid;
a GPCR agonist peptide comprising a Lys amino acid; and
a linker covalently coupling the conformationally modified albumin to the GPCR agonist peptide;
(a) wherein the linker is covalently bound to the GPCR agonist peptide via an amide bond formed by an epsilon amino group of the Lys amino acid;
(b) wherein the linker is covalently bound to the albumin via a covalent bond between a chiral carbon atom of a coupling group in the linker and a sulfur atom in the Cys₃₄ amino acid; and
(d) wherein the chiral carbon atom has a stereopreferred or stereoselective configuration.

10. The agonist fusion protein of claim 9, wherein the conformationally modified albumin is an at least partially defatted albumin.

11. The agonist fusion protein of claim 9, wherein the chiral carbon atom has a stereoselective configuration.

12. The agonist fusion protein of claim 9, wherein the linker is retro-Michael resistant.

13. The agonist fusion protein of claim 9, wherein the fusion protein is retro-Michael resistant albenatide (AB-013-AEEA-succinimide (SEQ ID NO:2)-albumin).

14. The agonist fusion protein of claim 9, wherein the fusion protein is retro-Michael resistant albenatidex (AB-014-AEEA-succinimide (SEQ ID NO:185)-albumin).

15. The agonist fusion protein of claim 9, wherein the fusion protein is retro-Michael resistant albugiptide (AB-029-AEEA-succinimide (SEQ ID NO:7)-albumin).

16. The agonist fusion protein of claim 9, wherein the fusion protein is retro-Michael resistant albutide (AB-044-AEEA-succinimide (SEQ ID NO:223)-albumin).

17. A pharmaceutical composition, comprising:
a pharmaceutically acceptable liquid carrier in combination with a Class B G protein-coupled receptor (GPCR) agonist fusion protein in which a GPCR agonist peptide is covalently coupled to albumin via a linker;
wherein the pharmaceutically acceptable liquid carrier is substantially free of unbound GPCR agonist peptide.

18. The pharmaceutical composition of claim 17, wherein the GPCR agonist fusion protein is selected from the group consisting of
(a) retro-Michael resistant albenatide (AB-013-AEEA-succinimide (SEQ ID NO:2)-albumin);
(b) retro-Michael resistant albenatidex (AB-014-AEEA-succinimide (SEQ ID NO: 185)-albumin);
(c) retro-Michael resistant albugiptide (AB-029-AEEA-succinimide (SEQ ID NO:7)-albumin); and
(d) retro-Michael resistant albutide (AB-044-AEEA-succinimide (SEQ ID NO: 223)-albumin).

19. The pharmaceutical composition of claim 17, wherein the liquid carrier has a pH of between pH 4.0-6.0.

20. The pharmaceutical composition of claim 17, wherein the composition is formulated such that a single dosage contains between 50-500 mcg of the GPCR agonist peptide.


SEQUENCE LISTING (471 sequences)

SEQ ID NO: 1 - protein, 31 aa, synthetic construct
  MOD_RES 31: note = AminoEthoxyEthoxyAcetic Acyl-MaleimidoPropionic Acyl
  SITE 2: note = D-alanine
HAEGTFTSDVSSYLEGQAAKEFIAWLVKGRK

SEQ ID NO: 2 - protein, 40 aa, synthetic construct
  MOD_RES 40: note = AminoEthoxyEthoxyAcetic Acyl-MaleimidoPropionic Acyl
HGEGTFTSDLSKQMEEEAVRLFIEWLKNGGPSSGAPPPSK

SEQ ID NO: 3 - protein, 39 aa, synthetic construct
HGEGTFTSDLSKQMEEEAVRLFIEWLKNGGPSSGAPPPS

SEQ ID NO: 4 - protein, 30 aa, synthetic construct
HAEGTFTSDVSSYLEGQAAKEFIAWLVKGR

SEQ ID NO: 5 - protein, 42 aa, synthetic construct
YAEGTFISDYSIAMDKIHQQDFVNWLLAQKGKKNDWKHNITQ

SEQ ID NO: 6 - protein, 29 aa, synthetic construct
HSQGTFTSDYSKYLDSRRAQDFVQWLMNT

SEQ ID NO: 7 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVSSYLEGQAAKEFIAWLVNGGPSSGAPPPSK

SEQ ID NO: 8 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAWLVKGRPSSGAPPPSK

SEQ ID NO: 9 - protein, 39 aa, synthetic construct
  MOD_RES 32: note = AminoEthoxyEthoxyAcetic Acyl-MaleimidoPropionic Acyl
HGEGTFTSDLSKQMEEEAVRLFIEWLKNGGPKSGAPPPS

SEQ ID NO: 10 - protein, 39 aa, synthetic construct
  SITE 2: note = D-alanine
  MOD_RES 12: note = AminoEthoxyEthoxyAcetic Acyl-MaleimidoPropionic Acyl
HAEGTFTSDVSKYLEGQAAKEFIAWLVKGRPSSGAPPPS

SEQ ID NO: 11 - protein, 39 aa, synthetic construct
  MOD_RES 16: note = AminoEthoxyEthoxyAcetic Acyl-MaleimidoPropionic Acyl
  SITE 2: note = D-alanine
HAEGTFTSDVSSYLEKQAAKEFIAWLVKGRPSSGAPPPS

SEQ ID NO: 12 - protein, 39 aa, synthetic construct; identical to SEQ ID NO: 9
  MOD_RES 32: note = AminoEthoxyEthoxyAcetic Acyl-MaleimidoPropionic Acyl

SEQ ID NO: 13 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDINKVLDTIAAKEFIAWLVKGRPSSGAPPPSK

SEQ ID NO: 14 - protein, 40 aa, synthetic construct
  MOD_RES 40: note = Palmitic acyl // Hexadecanoic acyl
  MOD_RES 2: note = Aib
  MOD_RES 20: note = Aib
YXEGTFTSDYSIYLDKQAAXEFVQWLLAGGPSSGAPPPSK

SEQ ID NO: 15 - protein, 40 aa, synthetic construct
  MOD_RES 40: note = AminoEthoxyEthoxyAcetic Acyl-MaleimidoPropionic Acyl
  SITE 30: note = D-alanine
YGGGTFTSDSFFYLELSHAKDFINWLQLGAPSSGAPPPSK

SEQ ID NO: 16 - protein, 40 aa, synthetic construct
  MOD_RES 2..3: note = Aib
  MOD_RES 20: note = Aib
  MOD_RES 28: note = Aib
  MOD_RES 40: note = AminoEthoxyEthoxyAcetic Acyl-MaleimidoPropionic Acyl
HXXGTFTSDEMNYLDDWMQXAFVNWLVXGIPSSGAPPPSK

SEQ ID NO: 17 - protein, 40 aa, synthetic construct
  MOD_RES 2: note = Aib
  MOD_RES 40: note = AminoEthoxyEthoxyAcetic Acyl-MaleimidoPropionic Acyl
FXPGTFTSDGHNYLDWQDAKEFIQWLGWGVPSSGAPPPSK

SEQ ID NO: 18 - protein, 40 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acyl-MaleimidoPropionic Acyl
YXMGTFTSDPQIXLEMKEQXDFINWLNDGFPSSGAPPPSK

SEQ ID NO: 19 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAPGTFTSDNHEXLDTYRQXDFINWLDTGVPSSGAPPPSK

SEQ ID NO: 20 - protein, 40 aa, synthetic construct
  SITE 2: note = D-alanine
  MOD_RES 40: note = AminoEthoxyEthoxyAcetic Acyl-MaleimidoPropionic Acyl
YANGTFTSDIWAYLDSSFAQDFVAWLYIGKPSSGAPPPSK

SEQ ID NO: 21 - protein, 40 aa, synthetic construct
  MOD_RES 2: note = Aib
  MOD_RES 40: note = AminoEthoxyEthoxyAcetic Acyl-MaleimidoPropionic Acyl
HXDGTFTSDFEHYLEDAVQKAFIAWLSTGVPSSGAPPPSK

SEQ ID NO: 22 - protein, 40 aa, synthetic construct
  MOD_RES 13: note = Aib
  MOD_RES 20: note = Aib
  MOD_RES 40: note = AminoEthoxyEthoxyAcetic Acyl-MaleimidoPropionic Acyl
FGDGTFTSDLRGXLEIMPQXAFVNWLTSGKPSSGAPPPSK

SEQ ID NO: 23 - protein, 40 aa, synthetic construct
  SITE 2..3: note = D-alanine
  SITE 10: note = D-serine
  MOD_RES 40: note = AminoEthoxyEthoxyAcetic Acyl-MaleimidoPropionic Acyl
HAAGTFTSDSMIYLDNAHQQEFIQWLFNGPPSSGAPPPSK

SEQ ID NO: 24 - protein, 40 aa, synthetic construct
  SITE 2: note = D-alanine
  SITE 10: note = D-serine
  MOD_RES 12: note = Aib
  SITE 16: note = D-alanine
  MOD_RES 20: note = Aib
  MOD_RES 40: note = AminoEthoxyEthoxyAcetic Acyl-MaleimidoPropionic Acyl
HAIGTFTSDSTXYLEAYKQXDFVQWLHSGGPSSGAPPPSK

SEQ ID NO: 25 - protein, 40 aa, synthetic construct
  SITE 2: note = D-serine
  SITE 10: note = D-alanine
  SITE 17: note = D-serine
  MOD_RES 40: note = AminoEthoxyEthoxyAcetic Acyl-MaleimidoPropionic Acyl
  SITE 30: note = D-alanine
YSWGTFTSDAGYYLDMSFQQAFIQWLKAGAPSSGAPPPSK

SEQ ID NO: 26 - protein, 40 aa, synthetic construct
  MOD_RES 13: note = Aib
  SITE 17: note = D-alanine
  SITE 28: note = D-serine
  MOD_RES 40: note = AminoEthoxyEthoxyAcetic Acyl-MaleimidoPropionic Acyl
YGRGTFTSDWIYXLDTAPQKEFVEWLHSGAPSSGAPPPSK

SEQ ID NO: 27 - protein, 40 aa, synthetic construct
  SITE 2: note = D-alanine
  SITE 10: note = D-serine
  MOD_RES 13: note = Aib
  MOD_RES 40: note = AminoEthoxyEthoxyAcetic Acyl-MaleimidoPropionic Acyl
YAMGTFTSDSEEXLEMVYQQAFVAWLPVGGPSSGAPPPSK

SEQ ID NO: 28 - protein, 40 aa, synthetic construct
  SITE 2..3: note = D-serine
  MOD_RES 13: note = Aib
  MOD_RES 17: note = Aib
  MOD_RES 20: note = Aib
  MOD_RES 40: note = AminoEthoxyEthoxyAcetic Acyl-MaleimidoPropionic Acyl
FSSGTFTSDWDWXLDIXIAXEFINWLVYGYPSSGAPPPSK

SEQ ID NO: 29 - protein, 40 aa, synthetic construct
  MOD_RES 2: note = Aib
  MOD_RES 40: note = AminoEthoxyEthoxyAcetic Acyl-MaleimidoPropionic Acyl
FXGGTFTSDDMRYLEPKGQQAFIQWLWVGQPSSGAPPPSK

SEQ ID NO: 30 - protein, 40 aa, synthetic construct
  MOD_RES 2: note = Aib
  MOD_RES 30: note = Aib
  MOD_RES 40: note = AminoEthoxyEthoxyAcetic Acyl-MaleimidoPropionic Acyl
YXSGTFTSDVETYLDLLIAKEFIAWLGAGXPSSGAPPPSK

SEQ ID NO: 31 - protein, 40 aa, synthetic construct
  MOD_RES 13: note = Aib
  MOD_RES 20: note = Aib
  MOD_RES 40: note = AminoEthoxyEthoxyAcetic Acyl-MaleimidoPropionic Acyl
HGRGTFTSDHQFXLDSMIQXEFIQWLHYGIPSSGAPPPSK

SEQ ID NO: 32 - protein, 40 aa, synthetic construct
  SITE 2: note = D-alanine
  MOD_RES 13: note = Aib
  SITE 16: note = D-serine
  SITE 30: note = D-alanine
  MOD_RES 40: note = AminoEthoxyEthoxyAcetic Acyl-MaleimidoPropionic Acyl
YARGTFTSDFINXLESGVAKAFVNWLQEGAPSSGAPPPSK

SEQ ID NO: 33 - protein, 40 aa, synthetic construct
  SITE 2: note = D-serine
  SITE 28: note = D-alanine
  SITE 30: note = D-alanine
  MOD_RES 40: note = AminoEthoxyEthoxyAcetic Acyl-MaleimidoPropionic Acyl
HSFGTFTSDGWMYLDMNEQKAFVAWLYAGAPSSGAPPPSK

SEQ ID NO: 34 - protein, 40 aa, synthetic construct
  SITE 2: note = D-serine
  MOD_RES 13: note = Aib
  MOD_RES 20: note = Aib
  MOD_RES 40: note = AminoEthoxyEthoxyAcetic Acyl-MaleimidoPropionic Acyl
FSRGTFTSDHYRXLDQSKAXEFIEWLESGNPSSGAPPPSK

SEQ ID NO: 35 - protein, 40 aa, synthetic construct
  SITE 2: note = D-serine
  MOD_RES 40: note = Aib
FSGGTFTSDHHRYLDQIPQKDFIQWLEFGKPSSGAPPPSK

SEQ ID NO: 36 - protein, 40 aa, synthetic construct
  SITE 2: note = D-alanine
  SITE 10: note = D-alanine
  MOD_RES 13: note = Aib
  SITE 18: note = D-alanine
  MOD_RES 40: note = AminoEthoxyEthoxyAcetic Acyl-MaleimidoPropionic Acyl
FAQGTFTSDANMXLDMFAQKAFINWLFQGGPSSGAPPPSK

SEQ ID NO: 37 - protein, 40 aa, synthetic construct
  SITE 2: note = D-alanine
  SITE 12: note = D-alanine
  MOD_RES 13: note = Aib
  MOD_RES 17: note = Aib
  MOD_RES 40: note = AminoEthoxyEthoxyAcetic Acyl-MaleimidoPropionic Acyl
FAAGTFTSDVTAXLEVXQQKAFIAWLNLGQPSSGAPPPSK

SEQ ID NO: 38 - protein, 40 aa, synthetic construct
  SITE 2: note = D-alanine
  MOD_RES 12: note = Aib
  SITE 16: note = D-serine
  MOD_RES 40: note = AminoEthoxyEthoxyAcetic Acyl-MaleimidoPropionic Acyl
HAIGTFTSDHMXYLDSDPAQDFIEWLMPGSPSSGAPPPSK

SEQ ID NO: 39 - protein, 40 aa, synthetic construct
  MOD_RES 2: note = Aib
  MOD_RES 40: note = AminoEthoxyEthoxyAcetic Acyl-MaleimidoPropionic Acyl
HXNGTFTSDIIHYLDPIVQKAFIEWLNGGAPSSGAPPPSK

SEQ ID NO: 40 - protein, 40 aa, synthetic construct
  MOD_RES 11: note = Aib
  MOD_RES 13: note = Aib
  MOD_RES 40: note = AminoEthoxyEthoxyAcetic Acyl-MaleimidoPropionic Acyl
HGEGTFTSDMXQXLDREIAKEFVEWLFFGYPSSGAPPPSK

SEQ ID NO: 41 - protein, 40 aa, synthetic construct
  SITE 2: note = D-serine
  SITE 12: note = D-serine
  MOD_RES 13: note = Aib
  SITE 28: note = D-serine
  MOD_RES 40: note = AminoEthoxyEthoxyAcetic Acyl-MaleimidoPropionic Acyl
HSPGTFTSDPDSXLDENHQQAFVAWLNSGMPSSGAPPPSK

SEQ ID NO: 42 - protein, 40 aa, synthetic construct
  SITE 2: note = D-serine
  SITE 3: note = D-alanine
  SITE 10: note = D-serine
  SITE 16..17: note = D-serine
  MOD_RES 20: note = Aib
  MOD_RES 40: note = AminoEthoxyEthoxyAcetic Acyl-MaleimidoPropionic Acyl
FSAGTFTSDSLDYLESSEQXEFINWLAAGVPSSGAPPPSK

SEQ ID NO: 43 - protein, 40 aa, synthetic construct
  SITE 2: note = D-alanine
  MOD_RES 3: note = Aib
  MOD_RES 16: note = Aib
  MOD_RES 20: note = Aib
  SITE 28: note = D-serine
  MOD_RES 40: note = AminoEthoxyEthoxyAcetic Acyl-MaleimidoPropionic Acyl
YAXGTFTSDKDPYLEXTYAXEFVAWLVSGRPSSGAPPPSK

SEQ ID NO: 44 - protein, 40 aa, synthetic construct
  MOD_RES 13: note = Aib
  MOD_RES 40: note = AminoEthoxyEthoxyAcetic Acyl-MaleimidoPropionic Acyl
FGRGTFTSDAVMXLDAMQAKAFIEWLWIGIPSSGAPPPSK

SEQ ID NO: 45 - protein, 40 aa, synthetic construct
  SITE 2..3: note = D-alanine
  MOD_RES 13: note = Aib
  MOD_RES 40: note = AminoEthoxyEthoxyAcetic Acyl-MaleimidoPropionic Acyl
HAAGTFTSDVDYXLEWKIQQDFIEWLSTGLPSSGAPPPSK

SEQ ID NO: 46 - protein, 40 aa, synthetic construct
  SITE 2: note = D-serine
  MOD_RES 18: note = Aib
  MOD_RES 20: note = Aib
  MOD_RES 40: note = AminoEthoxyEthoxyAcetic Acyl-MaleimidoPropionic Acyl
YSFGTFTSDIAGYLDLEXAXAFVQWLAHGKPSSGAPPPSK

SEQ ID NO: 47 - protein, 40 aa, synthetic construct
  SITE 2: note = D-serine
  MOD_RES 40: note = AminoEthoxyEthoxyAcetic Acyl-MaleimidoPropionic Acyl
HSHGTFTSDEVQYLDMDGQQEFINWLDLGYPSSGAPPPSK

SEQ ID NO: 48 - protein, 40 aa, synthetic construct
  SITE 2: note = D-serine
  MOD_RES 13: note = Aib
  MOD_RES 20: note = Aib
  MOD_RES 27: note = Aib
  MOD_RES 40: note = AminoEthoxyEthoxyAcetic Acyl-MaleimidoPropionic Acyl
HSRGTFTSDLQGXLEYADQXDFVNWLXTGFPSSGAPPPSK

SEQ ID NO: 49 - protein, 40 aa, synthetic construct
  MOD_RES 13: note = Aib
  SITE 16: note = D-alanine
  MOD_RES 27: note = D-alanine
  MOD_RES 40: note = AminoEthoxyEthoxyAcetic Acyl-MaleimidoPropionic Acyl
HGFGTFTSDGIDXLEAITAKEFIAWLAIGKPSSGAPPPSK

SEQ ID NO: 50 - protein, 40 aa, synthetic construct
  MOD_RES 2: note = Aib
  SITE 11: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HXTGTFTSDIAIXLDYKGAXAFVQWLDAGEPSSGAPPPSK

SEQ ID NO: 51 - protein, 40 aa, synthetic construct
  SITE 2: note = D-serine
  MOD_RES 11: note = Aib
  MOD_RES 13: note = Aib
  MOD_RES 20: note = Aib
  MOD_RES 40: note = AminoEthoxyEthoxyAcetic Acyl-MaleimidoPropionic Acyl
HSLGTFTSDLXMXLDVNIAXDFIAWLMIGYPSSGAPPPSK

SEQ ID NO: 52 - protein, 40 aa, synthetic construct
  MOD_RES 13: note = Aib
  SITE 28: note = D-serine
  MOD_RES 40: note = AminoEthoxyEthoxyAcetic Acyl-MaleimidoPropionic Acyl
YGDGTFTSDKHRXLEISQQKEFVQWLLSGLPSSGAPPPSK

SEQ ID NO: 53 - protein, 40 aa, synthetic construct
  SITE 2..3: note = D-alanine
  MOD_RES 13: note = Aib
  MOD_RES 28: note = Aib
  MOD_RES 30: note = Aib
  MOD_RES 40: note = AminoEthoxyEthoxyAcetic Acyl-MaleimidoPropionic Acyl
FAAGTFTSDQTTXLDAGSAKDFVNWLVXGXPSSGAPPPSK

SEQ ID NO: 54 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXGGTFTSDEPPYLDAWTQXEFVEWLVRGWPSSGAPPPSK

SEQ ID NO: 55 - protein, 40 aa, synthetic construct
  SITE 2: note = D-alanine
  MOD_RES 13: note = Aib
  SITE 17: note = D-alanine
  MOD_RES 40: note = AminoEthoxyEthoxyAcetic Acyl-MaleimidoPropionic Acyl
HAGGTFTSDGSRXLDIATQQDFIEWLLEGHPSSGAPPPSK

SEQ ID NO: 56 - protein, 40 aa, synthetic construct
  SITE 2: note = D-serine
  MOD_RES 40: note = AminoEthoxyEthoxyAcetic Acyl-MaleimidoPropionic Acyl
HSSGTFTSDPLKYLDHGNQQAFVNWLISGSPSSGAPPPSK

SEQ ID NO: 57 - protein, 40 aa, synthetic construct
  MOD_RES 2: note = Aib
  SITE 30: note = D-serine
  MOD_RES 40: note = AminoEthoxyEthoxyAcetic Acyl-MaleimidoPropionic Acyl
HXTGTFTSDISFYLEEYVAQEFVAWLQAGSPSSGAPPPSK

SEQ ID NO: 58 - protein, 40 aa, synthetic construct
  SITE 2: note = D-serine
  SITE 3: note = D-alanine
  SITE 10: note = D-alanine
  MOD_RES 13: note = Aib
  MOD_RES 17: note = Aib
  MOD_RES 40: note = AminoEthoxyEthoxyAcetic Acyl-MaleimidoPropionic Acyl
HSAGTFTSDAYEXLEPXDQKAFIAWLWHGLPSSGAPPPSK

SEQ ID NO: 59 - protein, 40 aa, synthetic construct
  SITE 2: note = D-serine
  MOD_RES 13: note = Aib
  SITE 17: note = D-serine
  MOD_RES 40: note = AminoEthoxyEthoxyAcetic Acyl-MaleimidoPropionic Acyl
YSRGTFTSDATLXLEGSKQKAFINWLLEGIPSSGAPPPSK

SEQ ID NO: 60 - protein, 40 aa, synthetic construct
  MOD_RES 13: note = Aib
  MOD_RES 40: note = AminoEthoxyEthoxyAcetic Acyl-MaleimidoPropionic Acyl
FGWGTFTSDLEKXLELARAQAFVEWLKVGSPSSGAPPPSK

SEQ ID NO: 61 - protein, 40 aa, synthetic construct
  MOD_RES 2: note = Aib
  SITE 18: note = D-alanine
  SITE 27: note = D-serine
  MOD_RES 40: note = AminoEthoxyEthoxyAcetic Acyl-MaleimidoPropionic Acyl
FXMGTFTSDPYPYLEWEAAQAFINWLSGGSPSSGAPPPSK

SEQ ID NO: 62 - protein, 40 aa, synthetic construct
  SITE 2: note = D-alanine
  MOD_RES 3: note = Aib
  MOD_RES 13: note = Aib
  MOD_RES 40: note = AminoEthoxyEthoxyAcetic Acyl-MaleimidoPropionic Acyl
HAXGTFTSDMPTXLDHIQQQDFVAWLVQGIPSSGAPPPSK

SEQ ID NO: 63 - protein, 40 aa, synthetic construct
  SITE 2: note = D-alanine
  SITE 3: note = D-serine
  MOD_RES 13: note = Aib
  MOD_RES 40: note = AminoEthoxyEthoxyAcetic Acyl-MaleimidoPropionic Acyl
FASGTFTSDHSDXLELKAQQEFVNWLRNGRPSSGAPPPSK

SEQ ID NO: 64 - protein, 40 aa, synthetic construct
  MOD_RES 40: note = AminoEthoxyEthoxyAcetic Acyl-MaleimidoPropionic Acyl
HGQGTFTSDYSKYLDARRAQDFVEWLKNGGPSSGAPPPSK

SEQ ID NO: 65 - protein, 40 aa, synthetic construct
  MOD_RES 2: note = Aib
  MOD_RES 13: note = Aib
  MOD_RES 40: note = AminoEthoxyEthoxyAcetic Acyl-MaleimidoPropionic Acyl
HXQGTFTSDYSKXLDKRRAKDFVEWLKNGGPSSGAPPPSK

SEQ ID NO: 66 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDKRRAKDFVEWLKNGGPSSGAPPPSK

SEQ ID NO: 67 - protein, 40 aa, synthetic construct
  MOD_RES 2: note = Aib
  MOD_RES 13: note = Alpha-Methyl-Leucine
  MOD_RES 20: note = Aib
  MOD_RES 40: note = AminoEthoxyEthoxyAcetic Acyl-MaleimidoPropionic Acyl
YXQGTFTSDYSIXLDKIAQXAFIEYLLEGGPSSGAPPPSK

SEQ ID NO: 68 - protein, 40 aa, synthetic construct
  MOD_RES 2: note = Aib
  MOD_RES 13: note = Aib
  MOD_RES 40: note = AminoEthoxyEthoxyAcetic Acyl-MaleimidoPropionic Acyl
YXEGTFTSDYSIXLDKIAQKAFVQWLIAGGPSSGAPPPSK

SEQ ID NO: 69 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAWLVNGGPSSGAPPPSK

SEQ ID NO: 70 - protein, 40 aa, synthetic construct; identical to SEQ ID NO: 14
  MOD_RES 2: note = Aib
  MOD_RES 20: note = Aib
  MOD_RES 40: note = AminoEthoxyEthoxyAcetic Acyl-MaleimidoPropionic Acyl

SEQ ID NO: 71 - protein, 40 aa, synthetic construct
  MOD_RES 2: note = Aib
  MOD_RES 20: note = Aib
  MOD_RES 35: note = Aib
  MOD_RES 40: note = AminoEthoxyEthoxyAcetic Acyl-MaleimidoPropionic Acyl
  SITE 29: note = D-alanine
HXHGTFTSDLSKLLEEQRQXEFIEWLKAAGPPPSXKPPPK

SEQ ID NO: 72 - protein, 39 aa, synthetic construct
  SITE 2: note = D-alanine
  MOD_RES 20: note = AminoEthoxyEthoxyAcetic Acyl-MaleimidoPropionic Acyl
HAMGTFTSDRHWYLDMSHQKAFVQWLAYGNPSSGAPPPS

SEQ ID NO: 73 - protein, 39 aa, synthetic construct
  SITE 2: note = D-alanine
  SITE 3: note = D-serine
  MOD_RES 20: note = AminoEthoxyEthoxyAcetic Acyl-MaleimidoPropionic Acyl
YASGTFTSDWGRYLELLIQKEFVNWLIIGAPSSGAPPPS

SEQ ID NO: 74 - protein, 39 aa, synthetic construct
  SITE 10: note = D-serine
  MOD_RES 13: note = Aib
  SITE 17: note = D-serine
  SITE 18: note = D-alanine
  SITE 27: note = D-alanine
  MOD_RES 20: note = AminoEthoxyEthoxyAcetic Acyl-AminoEthoxyEthoxyAcetic Acyl-MaleimidoPropionic Acyl
HGKGTFTSDSAIXLEVSAAKAFIEWLAHGDPSSGAPPPS

SEQ ID NO: 75 - protein, 39 aa, synthetic construct
  SITE 3: note = D-serine
  SITE 2: note = D-alanine
  MOD_RES 13: note = Aib
  SITE 27: note = D-alanine
  MOD_RES 20: note = AminoEthoxyEthoxyAcetic Acyl-MaleimidoPropionic Acyl
HASGTFTSDILEXLDQAAAKEFVEWLAHGFPSSGAPPPS

SEQ ID NO: 76 - protein, 39 aa, synthetic construct
  SITE 2: note = D-alanine
  MOD_RES 12..13: note = Aib
  MOD_RES 20: note = AminoEthoxyEthoxyAcetic Acyl-MaleimidoPropionic Acyl
HAYGTFTSDYMXXLDFLQQKDFVAWLFMGVPSSGAPPPS

SEQ ID NO: 77 - protein, 39 aa, synthetic construct
  MOD_RES 2: note = Aib
  SITE 3: note = D-serine
  MOD_RES 10: note = Aib
  MOD_RES 20: note = AminoEthoxyEthoxyAcetic Acyl-MaleimidoPropionic Acyl
HXSGTFTSDXAYYLDNTTAKDFIQWLDAGPPSSGAPPPS

SEQ ID NO: 78 - protein, 39 aa, synthetic construct
  MOD_RES 2: note = Aib
  SITE 11: note = D-alanine
  MOD_RES 16: note = Aib
  SITE 30: note = D-serine
  MOD_RES 20: note = AminoEthoxyEthoxyAcetic Acyl-MaleimidoPropionic Acyl
FXKGTFTSDDAVYLEXHRAKEFVEWLFGGSPSSGAPPPS

SEQ ID NO: 79 - protein, 39 aa, synthetic construct
  MOD_RES 2: note = Aib
  MOD_RES 13: note = Aib
  MOD_RES 27: note = Aib
  MOD_RES 20: note = AminoEthoxyEthoxyAcetic Acyl-MaleimidoPropionic Acyl
YXHGTFTSDLTVXLDEIKAKEFVEWLXVGPPSSGAPPPS

SEQ ID NO: 80 - protein, 39 aa, synthetic construct
  SITE 2: note = D-serine
  SITE 12: note = D-alanine
  MOD_RES 13: note = Aib
  MOD_RES 16: note = Aib
  MOD_RES 20: note = AminoEthoxyEthoxyAcetic Acyl-MaleimidoPropionic Acyl
FSWGTFTSDFPAXLEXMKAKDFVEWLLDGNPSSGAPPPS

SEQ ID NO: 81 - protein, 39 aa, synthetic construct
  MOD_RES 2: note = Aib
  MOD_RES 13: note = Aib
  SITE 28: note = D-serine
  MOD_RES 20: note = AminoEthoxyEthoxyAcetic Acyl-MaleimidoPropionic Acyl
HXKGTFTSDDADXLEWYRQKAFVQWLPSGIPSSGAPPPS

SEQ ID NO: 82 - protein, 39 aa, synthetic construct
  MOD_RES 2: note = Aib
  MOD_RES 13: note = Aib
  SITE 27: note = D-serine
  MOD_RES 20: note = AminoEthoxyEthoxyAcetic Acyl-MaleimidoPropionic Acyl
YXWGTFTSDSNSXLEAKMQKEFVNWLSQGFPSSGAPPPS

SEQ ID NO: 83 - protein, 39 aa, synthetic construct
  SITE 2: note = D-alanine
  SITE 18: note = D-serine
  MOD_RES 20: note = AminoEthoxyEthoxyAcetic Acyl-MaleimidoPropionic Acyl
FATGTFTSDDADYLELFSQKAFIAWLDNGVPSSGAPPPS

SEQ ID NO: 84 - protein, 39 aa, synthetic construct
  SITE 2: note = D-serine
  MOD_RES 13: note = Aib
  MOD_RES 17: note = Aib
  MOD_RES 20: note = AminoEthoxyEthoxyAcetic Acyl-MaleimidoPropionic Acyl
YSYGTFTSDWDAXLESXMQKEFVQWLFYGQPSSGAPPPS

SEQ ID NO: 85 - protein, 39 aa, synthetic construct
  SITE 2: note = D-alanine
  SITE 12: note = D-serine
  MOD_RES 13: note = Aib
  MOD_RES 20: note = AminoEthoxyEthoxyAcetic Acyl-MaleimidoPropionic Acyl
YANGTFTSDTNSXLDSTQAKEFVAWLVQGDPSSGAPPPS

SEQ ID NO: 86 - protein, 39 aa, synthetic construct
  MOD_RES 2: note = Aib
  MOD_RES 20: note = AminoEthoxyEthoxyAcetic Acyl-MaleimidoPropionic Acyl
FXIGTFTSDKQAYLEHPRQKAFVAWLDVGYPSSGAPPPS

SEQ ID NO: 87 - protein, 39 aa, synthetic construct
  MOD_RES 2: note = Aib
  MOD_RES 13: note = Aib
  SITE 30: note = D-serine
  MOD_RES 20: note = AminoEthoxyEthoxyAcetic Acyl-MaleimidoPropionic Acyl
HXRGTFTSDKFIXLEYHNAKEFVAWLYKGSPSSGAPPPS

SEQ ID NO: 88 - protein, 39 aa, synthetic construct
  SITE 10: note = D-serine
  MOD_RES 20: note = AminoEthoxyEthoxyAcetic Acyl-MaleimidoPropionic Acyl
FGHGTFTSDSIWYLENYSQKEFIEWLEKGPPSSGAPPPS

SEQ ID NO: 89 - protein, 39 aa, synthetic construct
  SITE 2: note = D-serine
  SITE 3: note = D-alanine
  MOD_RES 12: note = Aib
  SITE 27: note = D-alanine
  MOD_RES 20: note = AminoEthoxyEthoxyAcetic Acyl-MaleimidoPropionic Acyl
HSAGTFTSDIRXYLEIMLQKEFVEWLAEGVPSSGAPPPS

SEQ ID NO: 90 - protein, 39 aa, synthetic construct
  MOD_RES 2: note = Aib
  SITE 18: note = D-serine
  MOD_RES 20: note = AminoEthoxyEthoxyAcetic Acyl-MaleimidoPropionic Acyl
HXFGTFTSDMLNYLEENSQKEFVNWLQLGMPSSGAPPPS

SEQ ID NO: 91 - protein, 39 aa, synthetic construct
  MOD_RES 20: note = AminoEthoxyEthoxyAcetic Acyl-MaleimidoPropionic Acyl
FGHGTFTSDIWIYLEVQTAKDFINWLSWGEPSSGAPPPS

SEQ ID NO: 92 - protein, 39 aa, synthetic construct
  SITE 2: note = D-serine
  MOD_RES 20: note = AminoEthoxyEthoxyAcetic Acyl-MaleimidoPropionic Acyl
HSGGTFTSDSGPYLDKTDQKAFINWLPIGNPSSGAPPPS

SEQ ID NO: 93 - protein, 39 aa, synthetic construct
  SITE 2: note = D-serine
  SITE 11: note = D-serine
  MOD_RES 13: note = Aib
  MOD_RES 20: note = AminoEthoxyEthoxyAcetic Acyl-MaleimidoPropionic Acyl
YSIGTFTSDHSPXLDHLFAKDFVEWLENGDPSSGAPPPS

SEQ ID NO: 94 - protein, 39 aa, synthetic construct
  MOD_RES 13: note = Aib
  MOD_RES 18: note = Aib
  SITE 17: note = D-serine
  MOD_RES 20: note = AminoEthoxyEthoxyAcetic Acyl-MaleimidoPropionic Acyl
FGYGTFTSDKEGXLEQSXAKEFIQWLPHGPPSSGAPPPS

SEQ ID NO: 95 - protein, 39 aa, synthetic construct
  SITE 2: note = D-serine
  MOD_RES 11: note = Aib
  MOD_RES 13: note = Aib
  MOD_RES 20: note = AminoEthoxyEthoxyAcetic Acyl-MaleimidoPropionic Acyl
FSWGTFTSDFXSXLDTTRAKDFVEWLIRGNPSSGAPPPS

SEQ ID NO: 96 - protein, 39 aa, synthetic construct
  MOD_RES 2: note = Aib
  SITE 18: note = D-serine
  MOD_RES 28: note = Aib
  MOD_RES 20: note = AminoEthoxyEthoxyAcetic Acyl-MaleimidoPropionic Acyl
HXPGTFTSDHEKYLEMVSAKDFIAWLRXGDPSSGAPPPS

SEQ ID NO: 97 - protein, 39 aa, synthetic construct
  SITE 2: note = D-alanine
  SITE 12: note = D-alanine
  MOD_RES 13: note = Aib
  SITE 17: note = D-alanine
  MOD_RES 20: note = AminoEthoxyEthoxyAcetic Acyl-MaleimidoPropionic Acyl
YAAGTFTSDTIAXLDNAAAKDFIAWLIQGYPSSGAPPPS

SEQ ID NO: 98 - protein, 39 aa, synthetic construct
  MOD_RES 2: note = Aib
  MOD_RES 13: note = Aib
  MOD_RES 20: note = AminoEthoxyEthoxyAcetic Acyl-MaleimidoPropionic Acyl
YXLGTFTSDTIHXLEFEAQKDFINWLKAGEPSSGAPPPS

SEQ ID NO: 99 - protein, 39 aa, synthetic construct
  MOD_RES 2: note = Aib
  MOD_RES 27: note = Aib
  MOD_RES 20: note = AminoEthoxyEthoxyAcetic Acyl-MaleimidoPropionic Acyl
YXQGTFTSDAMIYLDTPDAKEFIAWLXIGGPSSGAPPPS

SEQ ID NO: 100 - protein, 39 aa, synthetic construct
  MOD_RES 11: note = Aib
  MOD_RES 13: note = Aib
  MOD_RES 17: note = Aib
  MOD_RES 20: note = AminoEthoxyEthoxyAcetic Acyl-MaleimidoPropionic Acyl
YGAGTFTSDHXGXLDIXQQKEFVQWLETGTPSSGAPPPS

SEQ ID NO: 101 - protein, 39 aa, synthetic construct
  SITE 2: note = D-alanine
  MOD_RES 20: note = AminoEthoxyEthoxyAcetic Acyl-MaleimidoPropionic Acyl
YAVGTFTSDLSEYLEMNIAKDFVQWLLVGGPSSGAPPPS

SEQ ID NO: 102 - protein, 39 aa, synthetic construct
  SITE 12: note = D-serine
  MOD_RES 20: note = AminoEthoxyEthoxyAcetic Acyl-MaleimidoPropionic Acyl
YGPGTFTSDRYSYLEQHMAKEFIQWLGPGHPSSGAPPPS

SEQ ID NO: 103 - protein, 39 aa, synthetic construct
  MOD_RES 2: note = Aib
  SITE 28: note = D-alanine
  MOD_RES 20: note = AminoEthoxyEthoxyAcetic Acyl-MaleimidoPropionic Acyl
YXRGTFTSDMRVYLEETLQKAFVQWLIAGDPSSGAPPPS

SEQ ID NO: 104 - protein, 39 aa, synthetic construct
  SITE 2: note = D-serine
  MOD_RES 3: note = Aib
  MOD_RES 13: note = Aib
  MOD_RES 20: note = AminoEthoxyEthoxyAcetic Acyl-MaleimidoPropionic Acyl
YSXGTFTSDFMEXLDKKIQKDFIQWLWYGSPSSGAPPPS

SEQ ID NO: 105 - protein, 39 aa, synthetic construct
  MOD_RES 2: note = Aib
  SITE 3: note = D-serine
  MOD_RES 27: note = Aib
  MOD_RES 20: note = AminoEthoxyEthoxyAcetic Acyl-MaleimidoPropionic Acyl
FXSGTFTSDELRYLEEMHQKAFIQWLXTGFPSSGAPPPS

SEQ ID NO: 106 - protein, 39 aa, synthetic construct
  SITE 27: note = D-serine
  MOD_RES 20: note = AminoEthoxyEthoxyAcetic Acyl-MaleimidoPropionic Acyl
YGPGTFTSDWAKYLEGRDAKAFINWLSQGSPSSGAPPPS

SEQ ID NO: 107 - protein, 39 aa, synthetic construct
  MOD_RES 13: note = Aib
  SITE 30: note = D-alanine
  MOD_RES 20: note = AminoEthoxyEthoxyAcetic Acyl-MaleimidoPropionic Acyl
HGVGTFTSDHHFXLEIIMAKAFIEWLWHGAPSSGAPPPS

SEQ ID NO: 108 - protein, 39 aa, synthetic construct
  SITE 18: note = D-alanine
  MOD_RES 20: note = AminoEthoxyEthoxyAcetic Acyl-MaleimidoPropionic Acyl
HGIGTFTSDVPAYLDPAAAKDFIQWLVSGGPSSGAPPPS

SEQ ID NO: 109 - protein, 39 aa, synthetic construct
  SITE 2: note = D-serine
  MOD_RES 13: note = Aib
  MOD_RES 28: note = Aib
  MOD_RES 20: note = AminoEthoxyEthoxyAcetic Acyl-MaleimidoPropionic Acyl
HSNGTFTSDRRKXLEPPIAKEFVQWLIXGIPSSGAPPPS

SEQ ID NO: 110 - protein, 39 aa, synthetic construct
  MOD_RES 2: note = Aib
  SITE 12: note = D-alanine
  MOD_RES 20: note = AminoEthoxyEthoxyAcetic Acyl-MaleimidoPropionic Acyl
FXTGTFTSDTMAYLDYTHAKDFIAWLIDGKPSSGAPPPS

SEQ ID NO: 111 - protein, 39 aa, synthetic construct
  SITE 2: note = D-alanine
  SITE 10: note = D-serine
  MOD_RES 20: note = AminoEthoxyEthoxyAcetic Acyl-MaleimidoPropionic Acyl
YATGTFTSDSVQYLEHPMQKAFVAWLHTGRPSSGAPPPS

SEQ ID NO: 112 - protein, 39 aa, synthetic construct
  MOD_RES 2: note = Aib
  MOD_RES 30: note = Aib
  MOD_RES 20: note = AminoEthoxyEthoxyAcetic Acyl-MaleimidoPropionic Acyl
YXTGTFTSDEEWYLDNWMQKAFVQWLSNGXPSSGAPPPS

SEQ ID NO: 113 - protein, 39 aa, synthetic construct
  SITE 2: note = D-serine
  MOD_RES 13: note = Aib
  MOD_RES 16: note = Aib
  MOD_RES 20: note = AminoEthoxyEthoxyAcetic Acyl-MaleimidoPropionic Acyl
YSIGTFTSDGNKXLEXGRAKAFIAWLQYGRPSSGAPPPS

SEQ ID NO: 114 - protein, 39 aa, synthetic construct
  SITE 2: note = D-serine
  MOD_RES 20: note = AminoEthoxyEthoxyAcetic Acyl-MaleimidoPropionic Acyl
YSPGTFTSDMYVYLEPGDAKEFVQWLKNGGPSSGAPPPS

SEQ ID NO: 115 - protein, 39 aa, synthetic construct
  SITE 2: note = D-alanine
  MOD_RES 13: note = Aib
  SITE 30: note = D-serine
  MOD_RES 20: note = AminoEthoxyEthoxyAcetic Acyl-MaleimidoPropionic Acyl
FSIGTFTSDLMAXLERAAQKEFVNWLIIGSPSSGAPPPS

SEQ ID NO: 116 - protein, 39 aa, synthetic construct
  MOD_RES 2: note = Aib
  MOD_RES 20: note = AminoEthoxyEthoxyAcetic Acyl-MaleimidoPropionic Acyl
YXNGTFTSDIEQYLEPMVQKEFVQWLSPGNPSSGAPPPS

SEQ ID NO: 117 - protein, 39 aa, synthetic construct
  SITE 12: note = D-serine
  MOD_RES 13: note = Aib
  MOD_RES 20: note = AminoEthoxyEthoxyAcetic Acyl-MaleimidoPropionic Acyl
YGWGTFTSDSFSXLDWMMAKAFIQWLIVGIPSSGAPPPS

SEQ ID NO: 118 - protein, 39 aa, synthetic construct
  SITE 2: note = D-serine
  MOD_RES 10: note = Aib
  MOD_RES 20: note = AminoEthoxyEthoxyAcetic Acyl-MaleimidoPropionic Acyl
HSPGTFTSDXTHYLDNDQQKDFVNWLPEGWPSSGAPPPS

SEQ ID NO: 119 - protein, 39 aa, synthetic construct
  SITE 3: note = D-alanine
  MOD_RES 13: note = Aib
  SITE 16: note = D-serine
  MOD_RES 20: note = AminoEthoxyEthoxyAcetic Acyl-MaleimidoPropionic Acyl
YGAGTFTSDNHTXLESFAAKEFIQWLNSGQPSSGAPPPS

SEQ ID NO: 120 - protein, 39 aa, synthetic construct
  SITE 2: note = D-alanine
  MOD_RES 13: note = Aib
  MOD_RES 20: note = AminoEthoxyEthoxyAcetic Acyl-MaleimidoPropionic Acyl
YAMGTFTSDRLRXLDQFSAKAFVNWLSWGEPSSGAPPPS

SEQ ID NO: 121 - protein, 39 aa, synthetic construct
  MOD_RES 2: note = Aib
  MOD_RES 12..13: note = Aib
  SITE 3: note = D-alanine
  SITE 27: note = D-alanine
  MOD_RES 20: note = AminoEthoxyEthoxyAcetic Acyl-MaleimidoPropionic Acyl
FXAGTFTSDSLXXLDHHNQKAFVEWLAPGLPSSGAPPPS

SEQ ID NO: 122 - protein, 39 aa, synthetic construct
  MOD_RES 16: note = AminoEthoxyEthoxyAcetic Acyl-MaleimidoPropionic Acyl
HGQGTFTSDYSKYLDKRRAQDFVEWLKNGGPSSGAPPPS

SEQ ID NO: 123 - protein, 39 aa, synthetic construct
  MOD_RES 2: note = Aib
  MOD_RES 13: note = Aib
  MOD_RES 20: note = AminoEthoxyEthoxyAcetic Acyl-MaleimidoPropionic Acyl
HXQGTFTSDYSKXLDKRRAKDFVEWLKNGGPSSGAPPPS

SEQ ID NO: 124 - protein, 39 aa, synthetic construct
  MOD_RES 2: note = Aib
  MOD_RES 16: note = AminoEthoxyEthoxyAcetic Acyl-MaleimidoPropionic Acyl
HXQGTFTSDYSKYLDKRRAQDFVEWLKNGGPSSGAPPPS

SEQ ID NO: 125 - protein, 39 aa, synthetic construct
  MOD_RES 2: note = Aib
  MOD_RES 13: note = Aib
  MOD_RES 20: note = AminoEthoxyEthoxyAcetic Acyl-MaleimidoPropionic Acyl
YXEGTFTSDYSIXLDKIAQKAFVQWLIAGGPSSGAPPPS

SEQ ID NO: 126 - protein, 39 aa, synthetic construct
  MOD_RES 2: note = Aib
  MOD_RES 13: note = Alpha-Methyl-Leucine
  MOD_RES 20: note = AminoEthoxyEthoxyAcetic Acyl-MaleimidoPropionic Acyl
YXQGTFTSDYSIXLDKIAQKAFIEYLLEGGPSSGAPPPS

SEQ ID NO: 127 - protein, 39 aa, synthetic construct
  SITE 2: note = D-alanine
  MOD_RES 20: note = AminoEthoxyEthoxyAcetic Acyl-MaleimidoPropionic Acyl
HAEGTFTSDYAKYLDARRAKEFIAWLVNGGPSSGAPPPS

SEQ ID NO: 128 - protein, 39 aa, synthetic construct
  SITE 2: note = D-alanine
  SITE 10: note = D-serine
  MOD_RES 20: note = Aib
  MOD_RES 17: note = AminoEthoxyEthoxyAcetic Acyl-MaleimidoPropionic Acyl
YAWGTFTSDSKDYLEFKWAXEFIQWLDPGDPSSGAPPPS

SEQ ID NO: 129 - protein, 39 aa, synthetic construct
  MOD_RES 2: note = Aib
  MOD_RES 13: note = Aib
  MOD_RES 17: note = AminoEthoxyEthoxyAcetic Acyl-MaleimidoPropionic Acyl
  MOD_RES 20: note = Aib
FXYGTFTSDINVXLDIKWAXAFINWLPGGIPSSGAPPPS

SEQ ID NO: 130 - protein, 39 aa, synthetic construct
  SITE 2..3: note = D-alanine
  MOD_RES 13: note = Aib
  MOD_RES 20: note = Aib
  MOD_RES 17: note = AminoEthoxyEthoxyAcetic Acyl-MaleimidoPropionic Acyl
FAAGTFTSDRQEXLDVKLAXEFVQWLVSGDPSSGAPPPS

SEQ ID NO: 131 - protein, 39 aa, synthetic construct
  SITE 2: note = D-alanine
  MOD_RES 13: note = Aib
  SITE 27: note = D-serine
  MOD_RES 17: note = AminoEthoxyEthoxyAcetic Acyl-MaleimidoPropionic Acyl
FARGTFTSDSMQXLETKRQQEFVNWLSMGVPSSGAPPPS

SEQ ID NO: 132 - protein, 39 aa, synthetic construct
  SITE 2: note = D-alanine
  MOD_RES 13: note = Aib
  MOD_RES 20: note = Aib
  MOD_RES 17: note = AminoEthoxyEthoxyAcetic Acyl-MaleimidoPropionic Acyl
YAHGTFTSDTNRXLESKAAXEFIAWLEVGAPSSGAPPPS

SEQ ID NO: 133 - protein, 39 aa, synthetic construct
  MOD_RES 2: note = Aib
  SITE 12: note = D-serine
  MOD_RES 13: note = Aib
  MOD_RES 20: note = Aib
  SITE 16: note = D-serine
  MOD_RES 17: note = AminoEthoxyEthoxyAcetic Acyl-MaleimidoPropionic Acyl
FXGGTFTSDFRSXLDSKIAXEFINWLFKGFPSSGAPPPS

SEQ ID NO: 134 - protein, 39 aa, synthetic construct
  MOD_RES 2: note = Aib
  MOD_RES 17: note = AminoEthoxyEthoxyAcetic Acyl-MaleimidoPropionic Acyl
YXNGTFTSDMMPYLEPKHQKDFVEWLTSGDPSSGAPPPS

SEQ ID NO: 135 - protein, 39 aa, synthetic construct
  SITE 2: note = D-alanine
  MOD_RES 20: note = Aib
  MOD_RES 17: note = AminoEthoxyEthoxyAcetic Acyl-MaleimidoPropionic Acyl
HAPGTFTSDIIHYLETKIAXEFIQWLKRGSPSSGAPPPS

SEQ ID NO: 136 - protein, 39 aa, synthetic construct
  SITE 2: note = D-serine
  MOD_RES 10: note = Aib
  MOD_RES 13: note = Aib
  MOD_RES 20: note = Aib
  SITE 28: note = D-alanine
  MOD_RES 17: note = AminoEthoxyEthoxyAcetic Acyl-MaleimidoPropionic Acyl
FSVGTFTSDXSAXLDLKIAXEFINWLFAGFPSSGAPPPS

SEQ ID NO: 137 - protein, 39 aa, synthetic construct
  MOD_RES 2: note = Aib
  SITE 3: note = D-alanine
  SITE 27: note = D-serine
  MOD_RES 17: note = AminoEthoxyEthoxyAcetic Acyl-MaleimidoPropionic Acyl
YXAGTFTSDGNMYLDKKHAKDFIEWLSSGPPSSGAPPPS

SEQ ID NO: 138 - protein, 39 aa, synthetic construct
  SITE 2: note = D-alanine
  SITE 11: note = D-serine
  MOD_RES 13: note = Aib
  MOD_RES 17: note = AminoEthoxyEthoxyAcetic Acyl-MaleimidoPropionic Acyl
YAIGTFTSDPSHXLEVKNQKEFVQWLNRGIPSSGAPPPS

SEQ ID NO: 139 - protein, 39 aa, synthetic construct
  SITE 2: note = D-serine
  MOD_RES 13: note = Aib
  MOD_RES 17: note = AminoEthoxyEthoxyAcetic Acyl-MaleimidoPropionic Acyl
FSTGTFTSDFVEXLEIKEQKAFIEWLAQGGPSSGAPPPS

SEQ ID NO: 140 - protein, 39 aa, synthetic construct
  SITE 18: note = D-alanine
  SITE 27: note = D-serine
  MOD_RES 17: note = AminoEthoxyEthoxyAcetic Acyl-MaleimidoPropionic Acyl
FGTGTFTSDFDGYLEDKAAKAFIQWLSGGMPSSGAPPPS

SEQ ID NO: 141 - protein, 39 aa, synthetic construct
  SITE 2: note = D-serine
  MOD_RES 13: note = Aib
  MOD_RES 20: note = Aib
  MOD_RES 17: note = AminoEthoxyEthoxyAcetic Acyl-MaleimidoPropionic Acyl
HSWGTFTSDWYIXLEEKNAXDFIAWLYAGYPSSGAPPPS

SEQ ID NO: 142 - protein, 39 aa, synthetic construct
  MOD_RES 13: note = Aib
  MOD_RES 27..28: note = Aib
  MOD_RES 17: note = AminoEthoxyEthoxyAcetic Acyl-MaleimidoPropionic Acyl
FGPGTFTSDKKFXLELKGAKAFIAWLXXGFPSSGAPPPS

SEQ ID NO: 143 - protein, 39 aa, synthetic construct
  SITE 2: note = D-serine
  SITE 10: note = D-alanine
  SITE 12: note = D-serine
  MOD_RES 20: note = Aib
  MOD_RES 28: note = Aib
  MOD_RES 17: note = AminoEthoxyEthoxyAcetic Acyl-MaleimidoPropionic Acyl
HSNGTFTSDANSYLEPKAAXDFVNWLDXGWPSSGAPPPS

SEQ ID NO: 144 - protein, 39 aa, synthetic construct
  SITE 2: note = D-serine
  MOD_RES 17: note = AminoEthoxyEthoxyAcetic Acyl-MaleimidoPropionic Acyl
FSTGTFTSDPWVYLDSKQQQAFVEWLHFGNPSSGAPPPS

SEQ ID NO: 145 - protein, 39 aa, synthetic construct
  SITE 2: note = D-alanine
  MOD_RES 13: note = Aib
  MOD_RES 20: note = Aib
  MOD_RES 17: note = AminoEthoxyEthoxyAcetic Acyl-MaleimidoPropionic Acyl
FAEGTFTSDWYPXLDLKSAXDFVQWLYGGPPSSGAPPPS

SEQ ID NO: 146 - protein, 39 aa, synthetic construct
  MOD_RES 13: note = Aib
  MOD_RES 17: note = AminoEthoxyEthoxyAcetic Acyl-MaleimidoPropionic Acyl
FGIGTFTSDTARXLDDKDQKEFVQWLNDGMPSSGAPPPS

SEQ ID NO: 147 - protein, 39 aa, synthetic construct
  MOD_RES 20: note = Aib
  SITE 30: note = D-serine
  MOD_RES 17: note = AminoEthoxyEthoxyAcetic Acyl-MaleimidoPropionic Acyl
FGYGTFTSDKSTYLDEKIQXDFVEWLNDGSPSSGAPPPS

SEQ ID NO: 148 - protein, 39 aa, synthetic construct
  MOD_RES 11: note = Aib
  MOD_RES 17: note = AminoEthoxyEthoxyAcetic Acyl-MaleimidoPropionic Acyl
HGSGTFTSDAXPYLDQKGAQDFIAWLDGGPPSSGAPPPS

SEQ ID NO: 149 - protein, 39 aa, synthetic construct
  SITE 2: note = D-alanine
  MOD_RES 13: note = Aib
  MOD_RES 17: note = AminoEthoxyEthoxyAcetic Acyl-MaleimidoPropionic Acyl
HAIGTFTSDPPVXLEQKHQQEFVAWLDPGLPSSGAPPPS

SEQ ID NO: 150 - protein, 39 aa, synthetic construct
  SITE 2: note = D-alanine
  MOD_RES 20: note = Aib
  SITE 27: note = D-alanine
  MOD_RES 17: note = AminoEthoxyEthoxyAcetic Acyl-MaleimidoPropionic Acyl
YAYGTFTSDVNKYLDPKTAXAFVEWLAQGIPSSGAPPPS

SEQ ID NO: 151 - protein, 39 aa, synthetic construct
  SITE 2: note = D-alanine
  MOD_RES 20: note = Aib
  MOD_RES 17: note = AminoEthoxyEthoxyAcetic Acyl-MaleimidoPropionic Acyl
FAIGTFTSDRREYLEPKEQXDFIEWLRDGGPSSGAPPPS

SEQ ID NO: 152 - protein, 39 aa, synthetic construct
  MOD_RES 17: note = AminoEthoxyEthoxyAcetic Acyl-MaleimidoPropionic Acyl
YGNGTFTSDAWIYLDDKLQKEFVQWLRLGNPSSGAPPPS

SEQ ID NO: 153 - protein, 39 aa, synthetic construct
  SITE 2: note = D-alanine
  SITE 10..11: note = D-alanine
  MOD_RES 13: note = Aib
  MOD_RES 20: note = Aib
  MOD_RES 27: note = Aib
  MOD_RES 17: note = AminoEthoxyEthoxyAcetic Acyl-MaleimidoPropionic Acyl
YAPGTFTSDAAAXLEDKYQXAFIAWLXQGWPSSGAPPPS

SEQ ID NO: 154 - protein, 39 aa, synthetic construct
  SITE 3: note = D-alanine
  SITE 28: note = D-serine
  MOD_RES 17: note = AminoEthoxyEthoxyAcetic Acyl-MaleimidoPropionic Acyl
YGAGTFTSDIRAYLDEKWAKAFVAWLVSGMPSSGAPPPS

SEQ ID NO: 155 - protein, 39 aa, synthetic construct
  MOD_RES 13: note = Aib
  MOD_RES 18: note = Aib
  MOD_RES 17: note = AminoEthoxyEthoxyAcetic Acyl-MaleimidoPropionic Acyl
YGWGTFTSDPINXLDLKXQKDFVNWLPMGAPSSGAPPPS

SEQ ID NO: 156 - protein, 39 aa, synthetic construct
  SITE 2: note = D-serine
  SITE 10: note = D-serine
  SITE 12: note = D-alanine
  MOD_RES 17: note = AminoEthoxyEthoxyAcetic Acyl-MaleimidoPropionic Acyl
HSNGTFTSDSYAYLERKDQKAFIEWLWSGPPSSGAPPPS

SEQ ID NO: 157 - protein, 39 aa, synthetic construct
  SITE 2: note = D-serine
  MOD_RES 28: note = Aib
  MOD_RES 17: note = AminoEthoxyEthoxyAcetic Acyl-MaleimidoPropionic Acyl
YSMGTFTSDQGKYLEAKSAQAFINWLMXGAPSSGAPPPS

SEQ ID NO: 158 - protein, 39 aa, synthetic construct
  SITE 2: note = D-alanine
  MOD_RES 13: note = Aib
  MOD_RES 16: note = Aib
  SITE 28: note = D-alanine
  MOD_RES 17: note = AminoEthoxyEthoxyAcetic Acyl-MaleimidoPropionic Acyl
YAFGTFTSDLTRXLDXKTAKAFIEWLDAGMPSSGAPPPS

SEQ ID NO: 159 - protein, 39 aa, synthetic construct
  MOD_RES 2: note = Aib
  MOD_RES 10: note = Aib
  MOD_RES 20: note = Aib
  MOD_RES 17: note = AminoEthoxyEthoxyAcetic Acyl-MaleimidoPropionic Acyl
YXKGTFTSDXIYYLDWKNAXAFVNWLHIGIPSSGAPPPS

SEQ ID NO: 160 - protein, 39 aa, synthetic construct
  MOD_RES 2: note = Aib
  SITE 11: note = D-serine
  MOD_RES 12..13: note = Aib
  MOD_RES 20: note = Aib
  MOD_RES 27: note = Aib
  MOD_RES 17: note = AminoEthoxyEthoxyAcetic Acyl-MaleimidoPropionic Acyl
HXQGTFTSDWSXXLEKKVQXEFVNWLXTGDPSSGAPPPS

SEQ ID NO: 161 - protein, 39 aa, synthetic construct
  SITE 2..3: note = D-serine
  SITE 12: note = D-alanine
  MOD_RES 13: note = Aib
  SITE 18: note = D-alanine
  MOD_RES 27: note = Aib
  MOD_RES 17: note = AminoEthoxyEthoxyAcetic Acyl-MaleimidoPropionic Acyl
FSSGTFTSDDRAXLDSKAAQAFVAWLXAGTPSSGAPPPS

SEQ ID NO: 162 - protein, 39 aa, synthetic construct
  SITE 2: note = D-alanine
  SITE 18: note = D-alanine
  SITE 30: note = D-serine
  MOD_RES 17: note = AminoEthoxyEthoxyAcetic Acyl-MaleimidoPropionic Acyl
YAIGTFTSDMKIYLDEKAAKAFVQWLANGSPSSGAPPPS

SEQ ID NO: 163 - protein, 39 aa, synthetic construct
  MOD_RES 13: note = Aib
  MOD_RES 20: note = Aib
  MOD_RES 17: note = AminoEthoxyEthoxyAcetic Acyl-MaleimidoPropionic Acyl
FGQGTFTSDYDQXLDNKPQXDFIEWLMYGEPSSGAPPPS

SEQ ID NO: 164 - protein, 39 aa, synthetic construct
  SITE 2: note = D-serine
  MOD_RES 13: note = Aib
  MOD_RES 17: note = AminoEthoxyEthoxyAcetic Acyl-MaleimidoPropionic Acyl
FSMGTFTSDLDRXLDWKIQKDFVQWLVSGTPSSGAPPPS

SEQ ID NO: 165 - protein, 39 aa, synthetic construct
  SITE 8: note = D-serine
  SITE 12: note = D-serine
  MOD_RES 17: note = AminoEthoxyEthoxyAcetic Acyl-MaleimidoPropionic Acyl
  SITE 2: note = D-serine
YSVGTFTSDLHSYLDLKGAQDFVNWLVWGQPSSGAPPPS

SEQ ID NO: 166 - protein, 39 aa, synthetic construct
  MOD_RES 2: note = Aib
  MOD_RES 17: note = AminoEthoxyEthoxyAcetic Acyl-MaleimidoPropionic Acyl
HXIGTFTSDDVYYLDLKPQKEFVEWLGLGSPSSGAPPPS

SEQ ID NO: 167 - protein, 39 aa, synthetic construct
  MOD_RES 13: note = Aib
  MOD_RES 17: note = AminoEthoxyEthoxyAcetic Acyl-MaleimidoPropionic Acyl
FGVGTFTSDVMIXLDIKEQKEFINWLQSGEPSSGAPPPS

SEQ ID NO: 168 - protein, 39 aa, synthetic construct
  MOD_RES 2: note = Aib
  MOD_RES 11: note = Aib
  SITE 12: note = D-serine
  SITE 16: note = D-serine
  SITE 30: note = D-serine
  MOD_RES 17: note = AminoEthoxyEthoxyAcetic Acyl-MaleimidoPropionic Acyl
HXFGTFTSDHXSYLESKMAQDFINWLIIGSPSSGAPPPS

SEQ ID NO: 169 - protein, 39 aa, synthetic construct
  SITE 2: note = D-alanine
  MOD_RES 30: note = Aib
  MOD_RES 17: note = AminoEthoxyEthoxyAcetic Acyl-MaleimidoPropionic Acyl
HAGGTFTSDPAKYLDSKVQQEFVAWLAFGXPSSGAPPPS

SEQ ID NO: 170 - protein, 39 aa, synthetic construct
  SITE 2: note = D-alanine
  SITE 11: note = D-serine
  MOD_RES 13: note = Aib
  SITE 28: note = D-serine
  MOD_RES 17: note = AminoEthoxyEthoxyAcetic Acyl-MaleimidoPropionic Acyl
YANGTFTSDVSFXLEYKDQKDFIQWLASGQPSSGAPPPS

SEQ ID NO: 171 - protein, 39 aa, synthetic construct
  SITE 2: note = D-serine
  MOD_RES 20: note = Aib
  MOD_RES 17: note = AminoEthoxyEthoxyAcetic Acyl-MaleimidoPropionic Acyl
FSVGTFTSDNRQYLDVKNQXDFIQWLPWGIPSSGAPPPS

SEQ ID NO: 172 - protein, 39 aa, synthetic construct
  MOD_RES 2: note = Aib
  MOD_RES 20: note = Aib
  MOD_RES 17: note = AminoEthoxyEthoxyAcetic Acyl-MaleimidoPropionic Acyl
HXVGTFTSDKETYLDIKKAXDFVEWLSTGRPSSGAPPPS

SEQ ID NO: 173 - protein, 39 aa, synthetic construct
  MOD_RES 13: note = Aib
  MOD_RES 20: note = Aib
  MOD_RES 17: note = AminoEthoxyEthoxyAcetic Acyl-MaleimidoPropionic Acyl
FGWGTFTSDDFNXLEYKHAXEFVNWLDVGQPSSGAPPPS

SEQ ID NO: 174 - protein, 39 aa, synthetic construct
  SITE 2: note = D-alanine
  MOD_RES 13: note = Aib
  MOD_RES 17: note = AminoEthoxyEthoxyAcetic Acyl-MaleimidoPropionic Acyl
FAYGTFTSDQSYXLDYKTAKDFIQWLTKGRPSSGAPPPS

SEQ ID NO: 175 - protein, 39 aa, synthetic construct
  MOD_RES 11: note = Aib
  SITE 16: note = D-alanine
  MOD_RES 17: note = AminoEthoxyEthoxyAcetic Acyl-MaleimidoPropionic Acyl
FGPGTFTSDDXIYLEAKNAQAFIEWLFSGQPSSGAPPPS

SEQ ID NO: 176 - protein, 39 aa, synthetic construct
  MOD_RES 2: note = Aib
  SITE 11: note = D-serine
  MOD_RES 13: note = Aib
  MOD_RES 20: note = Aib
  MOD_RES 17: note = AminoEthoxyEthoxyAcetic Acyl-MaleimidoPropionic Acyl
YXIGTFTSDSSIXLDSKAQXDFIQWLLFGQPSSGAPPPS

SEQ ID NO: 177 - protein, 39 aa, synthetic construct
  SITE 2: note = D-serine
  MOD_RES 13: note = Aib
  MOD_RES 17: note = AminoEthoxyEthoxyAcetic Acyl-MaleimidoPropionic Acyl
HSGGTFTSDPYGXLEQKMAKEFVEWLMTGKPSSGAPPPS

SEQ ID NO: 178 - protein, 39 aa, synthetic construct
  SITE 2: note = D-alanine
  MOD_RES 17: note = AminoEthoxyEthoxyAcetic Acyl-MaleimidoPropionic Acyl
HAEGTFTSDYAKYLDAKRAKEFIAWLVNGGPSSGAPPPS

SEQ ID NO: 179 - protein, 39 aa, synthetic construct
  MOD_RES 2: note = Aib
  MOD_RES 13: note = Alpha-Methyl-Leucine
  MOD_RES 20: note = Aib
  MOD_RES 17: note = AminoEthoxyEthoxyAcetic Acyl-MaleimidoPropionic Acyl
YXQGTFTSDYSIXLDKKAQXAFIEYLLEGGPSSGAPPPS

SEQ ID NO: 180 - protein, 39 aa, synthetic construct; identical to SEQ ID NO: 122
  MOD_RES 16: note = AminoEthoxyEthoxyAcetic Acyl-MaleimidoPropionic Acyl

SEQ ID NO: 181 - protein, 39 aa, synthetic construct
  MOD_RES 2: note = Aib
  MOD_RES 13: note = Aib
  MOD_RES 16: note = AminoEthoxyEthoxyAcetic Acyl-MaleimidoPropionic Acyl
HXQGTFTSDYSKXLDKRRAQDFVEWLKNGGPSSGAPPPS

SEQ ID NO: 182 - protein, 39 aa, synthetic construct; identical to SEQ ID NO: 124
  MOD_RES 2: note = Aib
  MOD_RES 16: note = AminoEthoxyEthoxyAcetic Acyl-MaleimidoPropionic Acyl

SEQ ID NO: 183 - protein, 40 aa, synthetic construct
  VARIANT 1: note = X can be H, Y or F
  VARIANT 2: note = X can be G, D-Alanine, Aib or D-serine
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y or Aib
  MOD_RES 13: note = Aib
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K or absent
XXXGTFTSDXXXXLXXXXXXXFXXWLXXGXPSSGAPPPSX

SEQ ID NO: 184 - protein, 41 aa, synthetic construct
  VARIANT 1: note = X can be H, Y or F
  VARIANT 2: note = X can be G, D-Alanine, Aib or D-serine
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Q, N-Methyl-L-Leucine or Aib
  MOD_RES 13: note = Aib
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K, Q, R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K or absent
  MOD_RES 2: note = Amino-cyclobutyl-1-carboxylic acid
  MOD_RES 13: note = N-Methyl-L-Leucine
  MOD_RES 41: note = AminoEthoxyEthoxyAcetic Acid // 8-amino-3,6-dioxa-octanoic acid
XXXGTFTSDXXXXLXXXXXXXFXXWLXXGXPSSGAPPPSXK

SEQ ID NO: 185 - protein, 31 aa, synthetic construct
  MOD_RES 2: note = Aib
  MOD_RES 31: note = AminoEthoxyEthoxyAcetic Acyl-MaleimidoPropionic Acyl
HXEGTFTSDVSSYLEGQAAKEFIAWLVKGRK

SEQ ID NO: 186 - protein, 31 aa, synthetic construct; identical to SEQ ID NO: 185
  MOD_RES 2: note = Aib
  MOD_RES 31: note = AminoEthoxyEthoxyAcetic Acyl-AminoEthoxyEthoxyAcetic Acyl-MaleimidoPropionic Acyl

SEQ ID NO: 187 - protein, 31 aa, synthetic construct; identical to SEQ ID NO: 185
  MOD_RES 2: note = Aib
  MOD_RES 31: note = AminoEthoxyEthoxyAcetic Acyl-8-AminoOctanoic Acyl-MaleimidoPropionic Acyl

SEQ ID NO: 188 - protein, 31 aa, synthetic construct; identical to SEQ ID NO: 185
  MOD_RES 2: note = Aib

SEQ ID NO: 189 - protein, 32 aa, synthetic construct
  MOD_RES 2: note = Aib
  MOD_RES 32: note = AminoEthoxyEthoxyAcetic Acyl-MaleimidoPropionic Acyl
  MOD_RES 31: note = AminoEthoxyEthoxyAcetic Acid // 8-amino-3,6-dioxa-octanoic acid
HXEGTFTSDVSSYLEGQAAKEFIAWLVKGRXK

SEQ ID NO: 190 - protein, 40 aa, synthetic construct; identical to SEQ ID NO: 2
  MOD_RES 40: note = AminoEthoxyEthoxyAcetic Acyl-AminoEthoxyEthoxyAcetic Acyl-MaleimidoPropionic Acyl

SEQ ID NO: 191 - protein, 40 aa, synthetic construct; identical to SEQ ID NO: 2
  MOD_RES 40: note = AminoEthoxyEthoxyAcetic Acyl-8-AminoOctanoic Acyl-MaleimidoPropionic Acyl

SEQ ID NO: 192 - protein, 40 aa, synthetic construct; identical to SEQ ID NO: 2

SEQ ID NO: 193 - protein, 41 aa, synthetic construct
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HGEGTFTSDLSKQMEEEAVRLFIEWLKNGGPSSGAPPPSXK

SEQ ID NO: 194 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYSKYLDSRRAQDFVQWLMNTGPSSGAPPPSK

SEQ ID NO: 195 - protein, 40 aa, synthetic construct; identical to SEQ ID NO: 194
  SITE 2: note = D-serine
  MOD_RES 40: note = AminoEthoxyEthoxyAcetic Acyl-AminoEthoxyEthoxyAcetic Acyl-MaleimidoPropionic Acyl

SEQ ID NO: 196 - protein, 40 aa, synthetic construct; identical to SEQ ID NO: 194
  SITE 2: note = D-serine
  MOD_RES 40: note = AminoEthoxyEthoxyAcetic Acyl-8-AminoOctanoic Acyl-MaleimidoPropionic Acyl

SEQ ID NO: 197 - protein, 40 aa, synthetic construct; identical to SEQ ID NO: 194
  SITE 2: note = D-serine

SEQ ID NO: 198 - protein, 41 aa, synthetic construct
  SITE 2: note = D-serine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HSQGTFTSDYSKYLDSRRAQDFVQWLMNTGPSSGAPPPSXK

SEQ ID NO: 199 - protein, 40 aa, synthetic construct
  SITE 2: note = D-alanine
  MOD_RES 40: note = AminoEthoxyEthoxyAcetic Acyl-MaleimidoPropionic Acyl
YAEGTFISDYSIAMDKIHQQDFVNWLLAQGPSSGAPPPSK

SEQ ID NO: 200 - protein, 40 aa, synthetic construct; identical to SEQ ID NO: 199
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 201 - protein, 40 aa, synthetic construct; identical to SEQ ID NO: 199
  SITE 2: note = D-alanine
  MOD_RES 40: note = AminoEthoxyEthoxyAcetic Acyl-8-AminoOctanoic Acyl-MaleimidoPropionic Acyl

SEQ ID NO: 202 - protein, 40 aa, synthetic construct; identical to SEQ ID NO: 199
  SITE 2: note = D-alanine

SEQ ID NO: 203 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
YAEGTFISDYSIAMDKIHQQDFVNWLLAQGPSSGAPPPSXK

SEQ ID NO: 204 - protein, 30 aa, synthetic construct
  MOD_RES 20: note = AminoEthoxyEthoxyAcetic Acyl-MaleimidoPropionic Acyl
HAEGTFTSDVSSYLEGQAAKEFIAWLVRGR

SEQ ID NO: 205 - protein, 31 aa, synthetic construct
  MOD_RES 20: note = AminoEthoxyEthoxyAcetic Acyl-MaleimidoPropionic Acyl
  MOD_RES 2: note = Aib
HXEGTFTSDVSSYLEGQAAKEFIAWLVRGRG

SEQ ID NO: 206 - protein, 31 aa, synthetic construct
  MOD_RES 24: note = AminoEthoxyEthoxyAcetic Acyl-MaleimidoPropionic Acyl
HVEGTFTSDVSSYLEEQAAREFIKWLVRGRG

SEQ ID NO: 207 - protein, 40 aa, synthetic construct; identical to SEQ ID NO: 7
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 208 - protein, 40 aa, synthetic construct; identical to SEQ ID NO: 7
  SITE 2: note = D-alanine
  MOD_RES 40: note = AminoEthoxyEthoxyAcetic Acyl-8-AminoOctanoic Acyl-MaleimidoPropionic Acyl

SEQ ID NO: 209 - protein, 40 aa, synthetic construct; identical to SEQ ID NO: 7
  SITE 2: note = D-alanine

SEQ ID NO: 210 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HAEGTFTSDVSSYLEGQAAKEFIAWLVNGGPSSGAPPPSXK

SEQ ID NO: 211 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVASYLEGQAAKEFIAWLVNGGPSSGAPPPSK

SEQ ID NO: 212 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYSIYLDKQAAXEFVNWLLAGGPSSGAPPPSK

SEQ ID NO: 213 - protein, 34 aa, synthetic construct
  MOD_RES 2: note = Aib
  MOD_RES 20: note = AminoEthoxyEthoxyAcetic Acyl-MaleimidoPropionic Acyl
HXQGTFTSDYSKYLDEKKAKEFVEWLLEGGPSSG

SEQ ID NO: 214 - protein, 29 aa, synthetic construct
  MOD_RES 2: note = Aib
  MOD_RES 17: note = AminoEthoxyEthoxyAcetic Acyl-MaleimidoPropionic Acyl
HXQGTFTSDYSKYLDEKAAKEFIQWLLQT

SEQ ID NO: 215 - protein, 30 aa, synthetic construct
  MOD_RES 2: note = Aib
HXQGTFTSDYSKYLDEKRAKEFVQWLMNTC

SEQ ID NO: 216 - protein, 29 aa, synthetic construct
  SITE 2: note = D-serine
  MOD_RES 10: note = AminoEthoxyEthoxyAcetic Acyl-MaleimidoPropionic Acyl
HSQGTFTSDKSKYLDARAAQDFVQWLLDT

SEQ ID NO: 217 - protein, 29 aa, synthetic construct
  MOD_RES 24: note = AminoEthoxyEthoxyAcetic Acyl-MaleimidoPropionic Acyl
  MOD_RES 2: note = Amino-cyclobutyl-1-carboxylic acid
HXQGTFTSDYSKYLDERAAKDFIKWLESA

SEQ ID NO: 218 - protein, 39 aa, synthetic construct
  SITE 2: note = D-serine
  MOD_RES 14: note = AminoEthoxyEthoxyAcetic Acyl-MaleimidoPropionic Acyl
HSQGTFTSDLSKQKESKAAQDFIEWLKAGGPSSGAPPPS

SEQ ID NO: 219 - protein, 30 aa, synthetic construct
  MOD_RES 10: note = AminoEthoxyEthoxyAcetic Acyl-MaleimidoPropionic Acyl
HSQGTFTSDKSEYLDSERARDFVAWLEAGG

SEQ ID NO: 220 - protein, 39 aa, synthetic construct
  MOD_RES 2: note = Aib
  MOD_RES 10: note = AminoEthoxyEthoxyAcetic Acyl-MaleimidoPropionic Acyl
HXQGTFTSDKSKYLDERAAQDFVQWLLDGGPSSGAPPPS

SEQ ID NO: 221 - protein, 39 aa, synthetic construct
  MOD_RES 10: note = AminoEthoxyEthoxyAcetic Acyl-MaleimidoPropionic Acyl
HSQGTFTSDKSKYLDERRAQDFVQWLLDGGPSSGAPPPS

SEQ ID NO: 222 - protein, 39 aa, synthetic construct
  MOD_RES 2: note = Aib
  MOD_RES 20: note = Aib
  MOD_RES 34: note = Aib
  MOD_RES 14: note = AminoEthoxyEthoxyAcetic Acyl-MaleimidoPropionic Acyl
HXHGTGTSDLSKLKEEQRQXEFIEWLKAAGPPSXKPPPK

SEQ ID NO: 223 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIAWLVNGGPSSGAPPPSK

SEQ ID NO: 224 - protein, 40 aa, synthetic construct; identical to SEQ ID NO: 223
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 225 - protein, 40 aa, synthetic construct; identical to SEQ ID NO: 223
  SITE 2: note = D-alanine
  MOD_RES 40: note = AminoEthoxyEthoxyAcetic Acyl-8-AminoOctanoic Acyl-MaleimidoPropionic Acyl

SEQ ID NO: 226 - protein, 40 aa, synthetic construct; identical to SEQ ID NO: 223
  SITE 2: note = D-alanine

SEQ ID NO: 227 - protein, 40 aa, synthetic construct; identical to SEQ ID NO: 69
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 228 - protein, 40 aa, synthetic construct; identical to SEQ ID NO: 69
  MOD_RES 40: note = AminoEthoxyEthoxyAcetic Acyl-8-AminoOctanoic Acyl-MaleimidoPropionic Acyl
  SITE 2: note = D-alanine

SEQ ID NO: 229 - protein, 40 aa, synthetic construct; identical to SEQ ID NO: 69
  SITE 2: note = D-alanine

SEQ ID NO: 230 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HAEGTFTSDYAKYLDARRAKEFIAWLVNGGPSSGAPPPSXK

SEQ ID NO: 231 - protein, 42 aa, synthetic construct; identical to SEQ ID NO: 5

SEQ ID NO: 232 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HAEGTFTSDVASYLEGQAAKEFIAWLVNGGPSSGAPPPSXK

SEQ ID NO: 233 - protein, 31 aa, synthetic construct; identical to SEQ ID NO: 205
  MOD_RES 2: note = Aib
  MOD_RES 20: note = AminoEthoxyEthoxyAcetic Acyl-AminoEthoxyEthoxyAcetic Acyl-MaleimidoPropionic Acyl

SEQ ID NO: 234 - protein, 32 aa, synthetic construct
  MOD_RES 2: note = Aib
  MOD_RES 32: note = AminoEthoxyEthoxyAcetic Acyl-MaleimidoPropionic Acyl
HXEGTFTSDVSSYLEGQAAKEFIAWLVRGRGK

SEQ ID NO: 235 - protein, 32 aa, synthetic construct
  MOD_RES 2: note = Aib
  MOD_RES 32: note = AminoEthoxyEthoxyAcetic Acyl-8-AminoOctanoic Acyl-MaleimidoPropionic Acyl
  MOD_RES 20: note = Aib
HXEGTFTSDVSSYLEGQAAXEFIAWLVRGRGK

SEQ ID NO: 236 - protein, 39 aa, synthetic construct; identical to SEQ ID NO: 125
  MOD_RES 2: note = Aib
  MOD_RES 13: note = Aib
  MOD_RES 20: note = AminoEthoxyEthoxyAcetic Acyl-8-AminoOctanoic Acyl-MaleimidoPropionic Acyl

SEQ ID NO: 237 - protein, 39 aa, synthetic construct; identical to SEQ ID NO: 125
  MOD_RES 2: note = Aib
  MOD_RES 13: note = Aib
  MOD_RES 20: note = AminoEthoxyEthoxyAcetic Acyl-AminoEthoxyEthoxyAcetic Acyl-MaleimidoPropionic Acyl

SEQ ID NO: 238 - protein, 40 aa, synthetic construct; identical to SEQ ID NO: 14
  MOD_RES 2: note = Aib
  MOD_RES 20: note = Aib
  MOD_RES 40: note = AminoEthoxyEthoxyAcetic Acyl-AminoEthoxyEthoxyAcetic Acyl-MaleimidoPropionic Acyl

SEQ ID NO: 239 - protein, 40 aa, synthetic construct; identical to SEQ ID NO: 14
  MOD_RES 2: note = Aib
  MOD_RES 20: note = Aib
  MOD_RES 40: note = AminoEthoxyEthoxyAcetic Acyl-8-AminoOctanoic Acyl-AminoEthoxyEthoxyAcetic Acyl-MaleimidoPropionic Acyl

SEQ ID NO: 240 - protein, 34 aa, synthetic construct; identical to SEQ ID NO: 213
  MOD_RES 2: note = Aib
  MOD_RES 20: note = AminoEthoxyEthoxyAcetic Acyl-AminoEthoxyEthoxyAcetic Acyl-MaleimidoPropionic Acyl

SEQ ID NO: 241 - protein, 34 aa, synthetic construct; identical to SEQ ID NO: 213
  MOD_RES 2: note = Aib
  MOD_RES 20: note = AminoEthoxyEthoxyAcetic Acyl-8-AminoOctanoic Acyl-MaleimidoPropionic Acyl

SEQ ID NO: 242 - protein, 34 aa, synthetic construct; identical to SEQ ID NO: 213
  MOD_RES 2: note = Aib
  MOD_RES 20: note = AminoEthoxyEthoxyAcetic Acyl-8-AminoOctanoic Acyl-AminoEthoxyEthoxyAcetic Acyl-MaleimidoPropionic Acyl

SEQ ID NO: 243 - protein, 35 aa, synthetic construct
  MOD_RES 2: note = Aib
  MOD_RES 35: note = AminoEthoxyEthoxyAcetic Acyl-MaleimidoPropionic Acyl
HXQGTFTSDYSKYLDEKKAKEFVEWLLEGGPSSGK

SEQ ID NO: 244 - protein, 29 aa, synthetic construct
  MOD_RES 2: note = Aib
  MOD_RES 17: note = AminoEthoxyEthoxyAcetic Acyl-AminoEthoxyEthoxyAcetic Acyl-MaleimidoPropionic Acyl
HXQGTFTSDYSKYLDEKAAKEFIQWLLQT

SEQ ID NO: 245 - protein, 29 aa, synthetic construct
  MOD_RES 2: note = Aib
  MOD_RES 17: note = AminoEthoxyEthoxyAcetic Acyl-8-AminoOctanoic Acyl-MaleimidoPropionic Acyl
HXQGTFTSDYSKYLDEKAAKEFIQWLLQT

SEQ ID NO: 246 - protein, 39 aa, synthetic construct; identical to SEQ ID NO: 218
  SITE 2: note = D-serine
  MOD_RES 14: note = AminoEthoxyEthoxyAcetic Acyl-AminoEthoxyEthoxyAcetic Acyl-MaleimidoPropionic Acyl

SEQ ID NO: 247 - protein, 39 aa, synthetic construct; identical to SEQ ID NO: 218
  SITE 2: note = D-serine
  MOD_RES 14: note = AminoEthoxyEthoxyAcetic Acyl-8-AminoOctanoic Acyl-AminoEthoxyEthoxyAcetic Acyl-MaleimidoPropionic Acyl

SEQ ID NO: 248 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDLSKQLESKAAQDFIEWLKAGGPSSGAPPPSK

SEQ ID NO: 249 - protein, 40 aa, synthetic construct; identical to SEQ ID NO: 248
  SITE 2: note = D-serine
  MOD_RES 40: note = AminoEthoxyEthoxyAcetic Acyl-8-AminoOctanoic Acyl-MaleimidoPropionic Acyl

SEQ ID NO: 250 - protein, 31 aa, synthetic construct
  MOD_RES 31: note = AminoEthoxyEthoxyAcetic Acyl-Bromo-MaleimidoPropionic Acyl//8-amino-3,6-dioxa-octanoic acyl-3-(3-Bromo-2,5-dioxopyrrol-1-yl)propanoic acyl
HSQGTFTSDLSEYLDSERARDFVAWLEAGGK

SEQ ID NO: 251 - protein, 39 aa, synthetic construct; identical to SEQ ID NO: 179
  MOD_RES 2: note = Aib
  MOD_RES 13: note = Alpha-Methyl-Leucine
  MOD_RES 17: note = AminoEthoxyEthoxyAcetic Acyl-AminoEthoxyEthoxyAcetic Acyl-MaleimidoPropionic Acyl
  MOD_RES 20: note = Aib

SEQ ID NO: 252 - protein, 39 aa, synthetic construct; identical to SEQ ID NO: 179
  MOD_RES 2: note = Aib
  MOD_RES 20: note = Aib
  MOD_RES 13: note = Alpha-Methyl-Leucine
  MOD_RES 17: note = AminoEthoxyEthoxyAcetic Acyl-8-AminoOctanoic Acyl-MaleimidoPropionic Acyl

SEQ ID NO: 253 - protein, 39 aa, synthetic construct; identical to SEQ ID NO: 179
  MOD_RES 2: note = Aib
  MOD_RES 20: note = Aib
  MOD_RES 13: note = Alpha-Methyl-Leucine
  MOD_RES 17: note = AminoEthoxyEthoxyAcetic Acyl-8-AminoOctanoic Acyl-AminoEthoxyEthoxyAcetic Acyl-MaleimidoPropionic Acyl

SEQ ID NO: 254 - protein, 40 aa, synthetic construct; identical to SEQ ID NO: 67
  MOD_RES 2: note = Aib
  MOD_RES 13: note = Alpha-Methyl-Leucine
  MOD_RES 20: note = Aib
  MOD_RES 40: note = AminoEthoxyEthoxyAcetic Acyl-AminoEthoxyEthoxyAcetic Acyl-MaleimidoPropionic Acyl

SEQ ID NO: 255 - protein, 40 aa, synthetic construct; identical to SEQ ID NO: 67
  MOD_RES 2: note = Aib
  MOD_RES 20: note = Aib
  MOD_RES 13: note = Alpha-Methyl-Leucine
  MOD_RES 40: note = AminoEthoxyEthoxyAcetic Acyl-8-AminoOctanoic Acyl-MaleimidoPropionic Acyl

SEQ ID NO: 256 - protein, 40 aa, synthetic construct; identical to SEQ ID NO: 67
  MOD_RES 2: note = Aib
  MOD_RES 20: note = Aib
  MOD_RES 13: note = Alpha-Methyl-Leucine
  MOD_RES 40: note = AminoEthoxyEthoxyAcetic Acyl-8-AminoOctanoic Acyl-AminoEthoxyEthoxyAcetic Acyl-MaleimidoPropionic Acyl

SEQ ID NO: 257 - protein, 39 aa, synthetic construct; identical to SEQ ID NO: 221
  MOD_RES 10: note = AminoEthoxyEthoxyAcetic Acyl-AminoEthoxyEthoxyAcetic Acyl-MaleimidoPropionic Acyl

SEQ ID NO: 258 - protein, 39 aa, synthetic construct; identical to SEQ ID NO: 221
  MOD_RES 10: note = AminoEthoxyEthoxyAcetic Acyl-8-AminoOctanoic Acyl-MaleimidoPropionic Acyl

SEQ ID NO: 259 - protein, 40 aa, synthetic construct
  MOD_RES 40: note = AminoEthoxyEthoxyAcetic Acyl-MaleimidoPropionic Acyl
HSQGTFTSDYSKYLDERRAQDFVQWLLDGGPSSGAPPPSK

SEQ ID NO: 260 - protein, 40 aa, synthetic construct; identical to SEQ ID NO: 259
  MOD_RES 40: note = AminoEthoxyEthoxyAcetic Acyl-Bromo-MaleimidoPropionic Acyl//8-amino-3,6-dioxa-octanoic acyl-3-(3-Bromo-2,5-dioxopyrrol-1-yl)propanoic acyl

SEQ ID NO: 261 - protein, 39 aa, synthetic construct; identical to SEQ ID NO: 222
  MOD_RES 2: note = Aib
  MOD_RES 34: note = Aib
  MOD_RES 14: note = AminoEthoxyEthoxyAcetic Acyl-AminoEthoxyEthoxyAcetic Acyl-MaleimidoPropionic Acyl
  SITE 29: note = D-alanine
  MOD_RES 20: note = Aib

SEQ ID NO: 262 - protein, 39 aa, synthetic construct
  MOD_RES 2: note = Aib
  MOD_RES 20: note = Aib
  MOD_RES 34: note = Aib
  SITE 29: note = D-alanine
  MOD_RES 39: note = AminoEthoxyEthoxyAcetic Acyl-MaleimidoPropionic Acyl
HXHGTGTSDLSKLLEEQRQXEFIEWLKAAGPPSXKPPPK

SEQ ID NO: 263 - protein, 40 aa, synthetic construct
  MOD_RES 2: note = Aib
  MOD_RES 20: note = Aib
  MOD_RES 34: note = Aib
  SITE 29: note = D-alanine
  MOD_RES 39: note = AminoEthoxyEthoxyAcetic Acid // 8-amino-3,6-dioxa-octanoic acid
  MOD_RES 40: note = AminoEthoxyEthoxyAcetic Acyl-MaleimidoPropionic Acyl
HXHGTGTSDLSKLLEEQRQXEFIEWLKAAGPPSXKPPPXK

SEQ ID NO: 264 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYAKYLDARRAXEFIAWLVNGGPSSGAPPPSK

SEQ ID NO: 265 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYAKYLDARRAXEFIAWLVNGGPSSGAPPPSK

SEQ ID NO: 266 - protein, 40 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acyl-MaleimidoPropionic Acyl
YXEGTFTSDYSIXLDKRRAXEFIAWLVNGGPSSGAPPPSK

SEQ ID NO: 267 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAEGTFTSDYSIXLDKRRAXEFIAWLVNGGPSSGAPPPSK

SEQ ID NO: 268 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
YAEGTFTSDYSIXLDKRRAXEFIAWLVNGGPSSGAPPPSK

SEQ ID NO: 269 - protein, 40 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acyl-MaleimidoPropionic Acyl
YXEGTFTSDYSIXLDKIAQXAFIAWLVNGGPSSGAPPPSK

SEQ ID NO: 270 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAEGTFTSDYSIXLDKIAQXAFIAWLVNGGPSSGAPPPSK

SEQ ID NO: 271 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
YAEGTFTSDYSIXLDKIAQXAFIAWLVNGGPSSGAPPPSK

SEQ ID NO: 272 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAEGTFTSDYSIXLDKIAQXAFVQWLIAGGPSSGAPPPSK

SEQ ID NO: 273 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
YAEGTFTSDYSIXLDKIAQXAFVQWLIAGGPSSGAPPPSK

SEQ ID NO: 274 - protein, 41 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
YXEGTFTSDYSIXLDKIAQXAFVQWLIAGGPSSGAPPPSXK

SEQ ID NO: 275 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYAKYLDARRAXEFVQWLIAGGPSSGAPPPSK

SEQ ID NO: 276 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYAKYLDARRAXEFVQWLIAGGPSSGAPPPSK

SEQ ID NO: 277 - protein, 40 aa, synthetic construct
  MOD_RES 2: note = Aib
  MOD_RES 40: note = AminoEthoxyEthoxyAcetic Acyl-MaleimidoPropionic Acyl
  MOD_RES 20: note = Aib
YXEGTFTSDYAKYLDARRAXEFVQWLIAGGPSSGAPPPSK

SEQ ID NO: 278 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYAKYLDKIAQXAFVQWLIAGGPSSGAPPPSK

SEQ ID NO: 279 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYAKYLDKIAQXAFVQWLIAGGPSSGAPPPSK

SEQ ID NO: 280 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYAKYLDKIAQXAFVQWLIAGGPSSGAPPPSK

SEQ ID NO: 281 - protein, 40 aa, synthetic construct
  MOD_RES 2: note = Aib
  MOD_RES 40: note = AminoEthoxyEthoxyAcetic Acyl-MaleimidoPropionic Acyl
YXQGTFTSDYAKYLDARRAKEFIAWLVNGGPSSGAPPPSK

SEQ ID NO: 282 - protein, 40 aa, synthetic construct
  SITE 2: note = D-alanine
  MOD_RES 40: note = AminoEthoxyEthoxyAcetic Acyl-MaleimidoPropionic Acyl
YAQGTFTSDYAKYLDARRAKEFIAWLVNGGPSSGAPPPSK

SEQ ID NO: 283 - protein, 40 aa, synthetic construct
  MOD_RES 2: note = Aib
  MOD_RES 13: note = Alpha-Methyl-Leucine
  MOD_RES 40: note = AminoEthoxyEthoxyAcetic Acyl-MaleimidoPropionic Acyl
YXQGTFTSDYSIXLDKRRAKEFIAWLVNGGPSSGAPPPSK

SEQ ID NO: 284 - protein, 40 aa, synthetic construct
  SITE 2: note = D-alanine
  MOD_RES 13: note = Alpha-Methyl-Leucine
  MOD_RES 40: note = AminoEthoxyEthoxyAcetic Acyl-MaleimidoPropionic Acyl
YAQGTFTSDYSIXLDKRRAKEFIAWLVNGGPSSGAPPPSK

SEQ ID NO: 285 - protein, 40 aa, synthetic construct
  SITE 2: note = D-alanine
  MOD_RES 13: note = Alpha-Methyl-Leucine
  MOD_RES 40: note = AminoEthoxyEthoxyAcetic Acyl-MaleimidoPropionic Acyl
HAQGTFTSDYSIXLDKRRAKEFIAWLVNGGPSSGAPPPSK

SEQ ID NO: 286 - protein, 40 aa, synthetic construct
  MOD_RES 2: note = Aib
  MOD_RES 13: note = Alpha-Methyl-Leucine
  MOD_RES 20: note = Aib
  MOD_RES 40: note = AminoEthoxyEthoxyAcetic Acyl-MaleimidoPropionic Acyl
YXQGTFTSDYSIXLDKIAQXAFIAWLVNGGPSSGAPPPSK

SEQ ID NO: 287 - protein, 40 aa, synthetic construct
  SITE 2: note = D-alanine
  MOD_RES 13: note = Alpha-Methyl-Leucine
  MOD_RES 20: note = Aib
  MOD_RES 40: note = AminoEthoxyEthoxyAcetic Acyl-MaleimidoPropionic Acyl
YAQGTFTSDYSIXLDKIAQXAFIAWLVNGGPSSGAPPPSK

SEQ ID NO: 288 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
HAQGTFTSDYSIXLDKIAQXAFIAWLVNGGPSSGAPPPSK

SEQ ID NO: 289 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
YAQGTFTSDYSIXLDKIAQXAFIEYLLEGGPSSGAPPPSK

SEQ ID NO: 290 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
HAQGTFTSDYSIXLDKIAQXAFIEYLLEGGPSSGAPPPSK

SEQ ID NO: 291 - protein, 41 aa, synthetic construct
  MOD_RES 2: note = Aib
  MOD_RES 20: note = Aib
  MOD_RES 13: note = Alpha-Methyl-Leucine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
YXQGTFTSDYSIXLDKIAQXAFIEYLLEGGPSSGAPPPSXK

SEQ ID NO: 292 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEYLLEGGPSSGAPPPSK

SEQ ID NO: 293 - protein, 40 aa, synthetic construct
  MOD_RES 40: note = AminoEthoxyEthoxyAcetic Acyl-MaleimidoPropionic Acyl
  MOD_RES 2: note = Aib
HXQGTFTSDYAKYLDARRAKEFIEYLLEGGPSSGAPPPSK

SEQ ID NO: 294 - protein, 40 aa, synthetic construct
  MOD_RES 2: note = Aib
  MOD_RES 40: note = AminoEthoxyEthoxyAcetic Acyl-MaleimidoPropionic Acyl
YXQGTFTSDYAKYLDARRAKEFIEYLLEGGPSSGAPPPSK

SEQ ID NO: 295 - protein, 40 aa, synthetic construct
  SITE 2: note = D-alanine
  MOD_RES 40: note = AminoEthoxyEthoxyAcetic Acyl-MaleimidoPropionic Acyl
YAQGTFTSDYAKYLDARRAKEFIEYLLEGGPSSGAPPPSK

SEQ ID NO: 296 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAQGTFTSDYAKYLDKIAQXAFIEYLLEGGPSSGAPPPSK

SEQ ID NO: 297 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
HXQGTFTSDYAKYLDKIAQXAFIEYLLEGGPSSGAPPPSK

SEQ ID NO: 298 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXQGTFTSDYAKYLDKIAQXAFIEYLLEGGPSSGAPPPSK

SEQ ID NO: 299 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAQGTFTSDYAKYLDKIAQXAFIEYLLEGGPSSGAPPPSK

SEQ ID NO: 300 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
HXQGTFTSDYSIXLDKIAQXAFIEYLLEGGPSSGAPPPSK

SEQ ID NO: 301 - protein, 40 aa, synthetic construct; identical to SEQ ID NO: 223
  SITE 2: note = D-alanine
  MOD_RES 40: note = AminoEthoxyEthoxyAcetic Acyl-8-AminoOctanoic Acyl-AminoEthoxyEthoxyAcetic Acyl-MaleimidoPropionic Acyl

SEQ ID NO: 302 - protein, 39 aa, synthetic construct; identical to SEQ ID NO: 178
  SITE 2: note = D-alanine
  MOD_RES 17: note = AminoEthoxyEthoxyAcetic Acyl-MaleimidoPropionic Acyl

SEQ ID NO: 303 - protein, 39 aa, synthetic construct; identical to SEQ ID NO: 178
  SITE 2: note = D-alanine
  MOD_RES 17: note = AminoEthoxyEthoxyAcetic Acyl-AminoEthoxyEthoxyAcetic Acyl-MaleimidoPropionic Acyl

SEQ ID NO: 304 - protein, 39 aa, synthetic construct; identical to SEQ ID NO: 178
  SITE 2: note = D-alanine
  MOD_RES 17: note = AminoEthoxyEthoxyAcetic Acyl-AminoEthoxyEthoxyAcetic Acyl-8-AminoOctanoic Acyl-MaleimidoPropionic Acyl

SEQ ID NO: 305 - protein, 39 aa, synthetic construct; identical to SEQ ID NO: 178
  SITE 2: note = D-alanine
  MOD_RES 17: note = AminoEthoxyEthoxyAcetic Acyl-8-AminoOctanoic Acyl-MaleimidoPropionic Acyl

SEQ ID NO: 306 - protein, 39 aa, synthetic construct; identical to SEQ ID NO: 127
  SITE 2: note = D-alanine
  MOD_RES 20: note = AminoEthoxyEthoxyAcetic Acyl-AminoEthoxyEthoxyAcetic Acyl-MaleimidoPropionic Acyl

SEQ ID NO: 307 - protein, 39 aa, synthetic construct; identical to SEQ ID NO: 127
  SITE 2: note = D-alanine
  MOD_RES 20: note = AminoEthoxyEthoxyAcetic Acyl-AminoEthoxyEthoxyAcetic Acyl-8-AminoOctanoic Acyl-MaleimidoPropionic Acyl

SEQ ID NO: 308 - protein, 39 aa, synthetic construct; identical to SEQ ID NO: 127
  SITE 2: note = D-alanine
  MOD_RES 20: note = AminoEthoxyEthoxyAcetic Acyl-8-AminoOctanoic Acyl-MaleimidoPropionic Acyl

SEQ ID NO: 309 - protein, 40 aa, synthetic construct; identical to SEQ ID NO: 223
  SITE 2: note = D-alanine
  MOD_RES 40: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl

SEQ ID NO: 310 - protein, 40 aa, synthetic construct; identical to SEQ ID NO: 223
  SITE 2: note = D-alanine
  MOD_RES 40: note = AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl

SEQ ID NO: 311 - protein, 40 aa, synthetic construct; identical to SEQ ID NO: 223
  SITE 2: note = D-alanine
  MOD_RES 40: note = AminoEthoxyEthoxyAcetic Acyl-AminoEthoxyEthoxyAcetic Acyl-gamma-glutamic acid 1-Amido 19-Phosphatidyl Nonadecanoic acyl

SEQ ID NO: 312 - protein, 40 aa, synthetic construct
  SITE 2: note = D-alanine
  MOD_RES 40: note = PolyEthylene Glycol
HAQGTFTSDYAKYLDARRAKEFIAWLVNGGPSSGAPPPSC

SEQ ID NO: 313 - protein, 39 aa, synthetic construct
  SITE 2: note = D-alanine
  MOD_RES 17: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl
HAQGTFTSDYAKYLDAKRAKEFIAWLVNGGPSSGAPPPS

SEQ ID NO: 314 - protein, 39 aa, synthetic construct; identical to SEQ ID NO: 313
  SITE 2: note = D-alanine
  MOD_RES 17: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl

SEQ ID NO: 315 - protein, 39 aa, synthetic construct; identical to SEQ ID NO: 313
  SITE 2: note = D-alanine
  MOD_RES 17: note = AminoEthoxyEthoxyAcetic Acyl-AminoEthoxyEthoxyAcetic Acyl-gamma-glutamic acid 1-Amido 19-Phosphatidyl Nonadecanoic acyl //

SEQ ID NO: 316 - protein, 39 aa, synthetic construct; identical to SEQ ID NO: 313
  SITE 2: note = D-alanine
  MOD_RES 17: note = N6-[1-O-(17-carboxyheptadecyl)-D-glucopyranuronoyl]

SEQ ID NO: 317 - protein, 39 aa, synthetic construct; identical to SEQ ID NO: 179
  MOD_RES 2: note = Aib
  MOD_RES 20: note = Aib
  MOD_RES 17: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl
  MOD_RES 13: note = Alpha-Methyl-Leucine

SEQ ID NO: 318 - protein, 33 aa, synthetic construct
HADGSFSDEMNTILDNLAARDFINWLIQTKITD

SEQ ID NO: 319 - protein, 28 aa, synthetic construct
HSDAVFTDNYTRLRKQMAVKKYLNSILN

SEQ ID NO: 320 - protein, 37 aa, synthetic construct [sequence omitted]

SEQ ID NO: 321 - protein, 29 aa, synthetic construct
YADAIFTNSYRKVLGQLSARKLLQDIMSR

SEQ ID NO: 322 - protein, 40 aa, synthetic construct; identical to SEQ ID NO: 183
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib or Amino-cyclobutyl-1-carboxylic acid
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K ,R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K, AminoEthoxyEthoxyAcetic Acid // 8-amino-3,6-dioxa-octanoic acid or absent
  MOD_RES 40: note = AminoEthoxyEthoxyAcetic Acid // 8-amino-3,6-dioxa-octanoic acid

SEQ ID NO: 323 - protein, 39 aa, synthetic construct
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  MOD_RES 2: note = Aib or Amino-cyclobutyl-1-carboxylic acid
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 2: note = Aib
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K ,R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
XXXGTFTSDXXXXLXXXXXXXFXXWLXXGXPSSGAPPPS

SEQ ID NO: 324 - protein, 11 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
  VARIANT 11: note = X can be K, AminoEthoxyEthoxyAcetic Acid // 8-amino-3,6-dioxa-octanoic acid or absent
  MOD_RES 11: note = AminoEthoxyEthoxyAcetic Acid // 8-amino-3,6-dioxa-octanoic acid
XPSSGAPPPSX

SEQ ID NO: 325 - protein, 12 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
  VARIANT 2: note = X can be Q or A
  VARIANT 3: note = X can be Q, K ,R or Aib
  MOD_RES 3: note = Aib
  VARIANT 4: note = X can be D, E or A
  VARIANT 6: note = X can be V or I
  VARIANT 7: note = X can be Q, A, E or N
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
XXXXFXXWLXXG

SEQ ID NO: 326 - protein, 17 aa, synthetic construct
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  MOD_RES 2: note = Aib or Amino-cyclobutyl-1-carboxylic acid
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
XXXGTFTSDXXXXLXXX

SEQ ID NO: 327 - protein, 11 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
XPSSGAPPPSK

SEQ ID NO: 328 - protein, 23 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
  VARIANT 2: note = X can be Q or A
  VARIANT 3: note = X can be Q, K ,R or Aib
  MOD_RES 3: note = Aib
  VARIANT 21: note = X can be D, E or A
  VARIANT 6: note = X can be V or I
  VARIANT 7: note = X can be Q, A, E or N
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 13: note = X can be A,R,N,D,E,Q,G,H,I,L,K,M,F,P,S,T,W,Y,D-alanine,Aib,D-serine or D-Lysine
  SITE 13: note = D-alanine,D-serine or D-Lysine
  MOD_RES 13: note = Aib
  VARIANT 23: note = X can be K, AminoEthoxyEthoxyAcetic Acid // 8-amino-3,6-dioxa-octanoic acid or absent
  MOD_RES 23: note = AminoEthoxyEthoxyAcetic Acid // 8-amino-3,6-dioxa-octanoic acid
  VARIANT 4: note = X can be D, E or A
XXXXFXXWLXXGXPSSGAPPPSX

SEQ ID NO: 329 - protein, 40 aa, synthetic construct; identical to SEQ ID NO: 183
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib or Amino-cyclobutyl-1-carboxylic acid
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysin
  SITE 18: note = D-alanine,D-serine or D-Lysine
  MOD_RES 18: note = Aib
  VARIANT 19: note = X can be Q or A
  VARIANT 20: note = X can be Q, K ,R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K, AminoEthoxyEthoxyAcetic Acid // 8-amino-3,6-dioxa-octanoic acid or absent
  MOD_RES 40: note = AminoEthoxyEthoxyAcetic Acid // 8-amino-3,6-dioxa-octanoic acid

SEQ ID NO: 330 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVSSYLEGQAAKEFIAWLVKGRPSSGAPPPSK

SEQ ID NO: 331 - protein, 40 aa, synthetic construct; identical to SEQ ID NO: 330
  MOD_RES 40: note = AminoEthoxyEthoxyAcetic Acyl-MaleimidoPropionic Acyl

SEQ ID NO: 332 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDVSSYLEGQAAKEFIAWLVNGGK

SEQ ID NO: 333 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDINKVLDTIAAKEFIAWLVKGRK

SEQ ID NO: 334 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDAEKAKEAEKAKEFIAWLVKGRK

SEQ ID NO: 335 - protein, 31 aa, synthetic construct
  SITE 2: note = D-alanine
  MOD_RES 31: note = AminoEthoxyEthoxyAcetic Acyl-MaleimidoPropionic Acyl
  MOD_RES 10: note = Aib
  MOD_RES 13: note = Aib
  MOD_RES 16: note = Aib
HAEGTFTSDXEKXKEXEKAKEFIAWLVKGRK

SEQ ID NO: 336 - protein, 40 aa, synthetic construct
  SITE 2: note = D-alanine
  MOD_RES 10: note = Aib
  MOD_RES 13: note = Aib
  MOD_RES 16: note = Aib
  MOD_RES 19: note = Aib
  MOD_RES 40: note = AminoEthoxyEthoxyAcetic Acyl-MaleimidoPropionic Acyl
HAEGTFTSDXESXKEXEKXKEFIAWLVKGRPSSGAPPPSK

SEQ ID NO: 337 - protein, 40 aa, synthetic construct
  SITE 2: note = D-alanine
  MOD_RES 11: note = Aib
  MOD_RES 14: note = Aib
  MOD_RES 18: note = Aib
  MOD_RES 40: note = AminoEthoxyEthoxyAcetic Acyl-MaleimidoPropionic Acyl
HAEGTFTSDVXSYXEGQXAKEFIAWLVKGRPSSGAPPPSK

SEQ ID NO: 338 - protein, 39 aa, synthetic construct
  SITE 2: note = D-alanine
  MOD_RES 17: note = AminoEthoxyEthoxyAcetic Acyl-MaleimidoPropionic Acyl
HAEGTFTSDVSSYLEGKAAKEFIAWLVKGRPSSGAPPPS

SEQ ID NO: 339 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVSSYLEGQAAKEFIAWLVKGRLLLLLLLLLK

SEQ ID NO: 340 - protein, 42 aa, synthetic construct
  SITE 2: note = D-alanine
HAEGTFTSDVSSYLEGQAAKEFIAWLVKGRPEAPTDPEAPTD

SEQ ID NO: 341 - protein, 40 aa, synthetic construct
  MOD_RES 40: note = AminoEthoxyEthoxyAcetic Acyl-MaleimidoPropionic Acyl
HGEGTFTSDLSKQMEEEAVRLFIEWLKNGGGGGGGGGGGK

SEQ ID NO: 342 - protein, 31 aa, synthetic construct; identical to SEQ ID NO: 1
  SITE 2: note = D-alanine
  MOD_RES 31: note = (AminoEthoxyEthoxyAcetic Acyl)4-3-MaleimidoPropionic Acyl

SEQ ID NO: 343 - protein, 30 aa, synthetic construct
  SITE 2: note = D-alanine
  MOD_RES 17: note = AminoEthoxyEthoxyAcetic Acyl-MaleimidoPropionic Acyl
HAEGTFTSDVSSYLEGKAAKEFIAWLVKGR

SEQ ID NO: 344 - protein, 31 aa, synthetic construct; identical to SEQ ID NO: 1
  SITE 2: note = D-alanine
  MOD_RES 31: note = 3-MaleimidoPropionic Acid // 3-(2,5-Dioxo-2,5-dihydro-1H-pyrrol-1-yl)propanoic Acid

SEQ ID NO: 345 - protein, 37 aa, synthetic construct
HSQGTFTSDYSKYLDSRRAQDFVQWLMNTKRNKNNIA

SEQ ID NO: 346 - protein, 41 aa, synthetic construct
  SITE 2: note = D-alanine
HAEGTFTSDVSSYLEGQAAKEFIAWLVKGPEAPTDPEAPTD

SEQ ID NO: 347 - protein, 31 aa, synthetic construct; identical to SEQ ID NO: 1
  SITE 2: note = D-alanine
  MOD_RES 31: note = (AminoEthoxyEthoxyAcetic Acyl)4-3-MaleimidoPropionic Acyl

SEQ ID NO: 348 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDINKVLDIIAAKEFIAWLVKGRPSSGAPPPSK

SEQ ID NO: 349 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDINKVLDIIAAKEFIAWLVKGRK

SEQ ID NO: 350 - protein, 40 aa, synthetic construct; identical to SEQ ID NO: 2
  MOD_RES 40: note = 3-MaleimidoPropionic Acid // 3-(2,5-Dioxo-2,5-dihydro-1H-pyrrol-1-yl)propanoic Acid

SEQ ID NO: 351 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDKIRAQEFVAWLMNGGPSSGAPPPSK

SEQ ID NO: 352 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDKIAAQDFVAWLLNGGPSSGAPPPSK

SEQ ID NO: 353 - protein, 40 aa, synthetic construct
  MOD_RES 40: note = AminoEthoxyEthoxyAcetic Acyl-MaleimidoPropionic Acyl
  MOD_RES 2: note = Aib
HXQGTFTSDYAKYLDKIAAQDFVAWLLDGGPSSGAPPPSK

SEQ ID NO: 354 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDKIAAQDFVAWLLDGGPSSGAPPPSK

SEQ ID NO: 355 - protein, 40 aa, synthetic construct
  MOD_RES 2: note = Aib
  MOD_RES 40: note = AminoEthoxyEthoxyAcetic Acyl-AminoEthoxyEthoxyAcetic Acyl-MaleimidoPropionic Acyl
HXQGTFTSDYSKYLDKIAAQDFVAYLLDGGPSSGAPPPSK

SEQ ID NO: 356 - protein, 40 aa, synthetic construct
  MOD_RES 40: note = AminoEthoxyEthoxyAcetic Acyl-AminoEthoxyEthoxyAcetic Acyl-MaleimidoPropionic Acyl
  MOD_RES 2: note = Aib
HXQGTFTSDLSKYLDEIAVQDFIEWLLDGGPSSGAPPPSK

SEQ ID NO: 357 - protein, 37 aa, synthetic construct
HSQGTFTSDYSKYLDSRRAQDFVQWLMNTKRNRNNIA

SEQ ID NO: 358 - protein, 34 aa, synthetic construct
IKPEAPGEDASPEELNRYYASLRHYLNLVTRQRY

SEQ ID NO: 359 - protein, 31 aa, synthetic construct
  MOD_RES 2: note = Aib
HXEGTFTSDVSSYLEGQALRHYINWLTRQRY

SEQ ID NO: 360 - protein, 45 aa, synthetic construct
HGEGTFTSDLSKQMEEEAVRLFIEWLKNGGPSSRHYLNLVTRQRY

SEQ ID NO: 361 - protein, 51 aa, synthetic construct
  SITE 2: note = D-Serine
HXQGTFTSDLSKYLEEEAVREFIAWLKNGGPSSGAPPPSRHYLNLVTRQRY

SEQ ID NO: 362 - protein, 39 aa, synthetic construct
  SITE 2: note = D-Serine
HXQGTYTNDVSKYXDSRRAQDFIEWLKNGGPSSGAPPPS

SEQ ID NO: 363 - protein, 40 aa, synthetic construct
  SITE 2: note = D-Serine
HXQGTYTNDVSKYKDSRRAQDFIEWLKNGGPSSGAPPPSC

SEQ ID NO: 364 - protein, 40 aa, synthetic construct
  SITE 2: note = D-Serine
HXQGTFTSDLSKQKDSRRAQDFIEWLKNGGPSSGAPPPSC

SEQ ID NO: 365 - protein, 40 aa, synthetic construct
  SITE 2: note = D-Serine
HXQGTYTNDVSKYXDSRRAQDFIEWLKNGGPSSGAPPPSC

SEQ ID NO: 366 - protein, 34 aa, synthetic construct
PKPEAPGKDASPEEWNRYYADLRHYLNWLTRQRY

SEQ ID NO: 367 - protein, 34 aa, synthetic construct
IKPEAPGEDASPEELNRYYASLRHYLNWVTRQXY

SEQ ID NO: 368 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYSIYLDKQAAXAFVQWLIAGGPSSGAPPPSK

SEQ ID NO: 369 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYSIYLDKQAAXAFVQWLLAGGPSSGAPPPSK

SEQ ID NO: 370 - protein, 40 aa, synthetic construct
  MOD_RES 40: note = AminoEthoxyEthoxyAcetic Acyl-MaleimidoPropionic Acyl
  MOD_RES 2: note = Aib
  MOD_RES 20: note = Aib
YXEGTFTSDYSIYLDKQAAXEFVQWLIAGGPSSGAPPPSK

SEQ ID NO: 371 - protein, 40 aa, synthetic construct
  SITE 2: note = D-alanine
  MOD_RES 40: note = AminoEthoxyEthoxyAcetic Acyl-MaleimidoPropionic Acyl
  MOD_RES 20: note = Aib
HAEGTFTSDYSIYLDKQAAXEFVQWLLAGGPSSGAPPPSK

SEQ ID NO: 372 - protein, 40 aa, synthetic construct
  MOD_RES 2: note = D-alanine
  MOD_RES 40: note = AminoEthoxyEthoxyAcetic Acyl-MaleimidoPropionic Acyl
  MOD_RES 20: note = Aib
YAEGTFTSDYSIYLDKQAAXEFVQWLLAGGPSSGAPPPSK

SEQ ID NO: 373 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
HXEGTFTSDYSIYLDKQAAXEFVQWLLAGGPSSGAPPPSK

SEQ ID NO: 374 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYSIYLDKQAAXAFVQWLIAGGPSSGAPPPSK

SEQ ID NO: 375 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYSIYLDKQAAXAFVQWLLAGGPSSGAPPPSK

SEQ ID NO: 376 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYSIYLDKQAAXEFVQWLIAGGPSSGAPPPSK

SEQ ID NO: 377 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYSIYLDKQAAXAFVQWLIAGGPSSGAPPPSK

SEQ ID NO: 378 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYSIYLDKQAAXAFVQWLLAGGPSSGAPPPSK

SEQ ID NO: 379 - protein, 40 aa, synthetic construct
  MOD_RES 20: note = Aib
  SITE 2: note = D-alanine
  MOD_RES 40: note = AminoEthoxyEthoxyAcetic Acyl-MaleimidoPropionic Acyl
YAEGTFTSDYSIYLDKQAAXEFVQWLIAGGPSSGAPPPSK

SEQ ID NO: 380 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYAKYLDARRAKEFVQWLLAGGPSSGAPPPSK

SEQ ID NO: 381 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDARRAKEFVQWLLAGGPSSGAPPPSK

SEQ ID NO: 382 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDKRRAKEFVQWLLAGGPSSGAPPPSK

SEQ ID NO: 383 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFVQWLLAGGPSSGAPPPSK

SEQ ID NO: 384 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDARRAKEFVQWLLAGGPSSGAPPPSK

SEQ ID NO: 385 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDKRRAKEFVQWLLAGGPSSGAPPPSK

SEQ ID NO: 386 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYAKYLDARRAKEFVQWLLAGGPSSGAPPPSK

SEQ ID NO: 387 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDARRAKEFVQWLLAGGPSSGAPPPSK

SEQ ID NO: 388 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDKRRAKEFVQWLLAGGPSSGAPPPSK

SEQ ID NO: 389 - protein, 40 aa, synthetic construct
  MOD_RES 40: note = AminoEthoxyEthoxyAcetic Acyl-MaleimidoPropionic Acyl
  MOD_RES 2: note = Aib
HXEGTFTSDYAKYLDARRAKEFVQWLLAGGPSSGAPPPSK

SEQ ID NO: 390 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDARRAKEFVQWLLAGGPSSGAPPPSK

SEQ ID NO: 391 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDKRRAKEFVQWLLAGGPSSGAPPPSK

SEQ ID NO: 392 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYAKYLDARRAKAFVQWLIAGGPSSGAPPPSK

SEQ ID NO: 393 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDARRAKAFVQWLIAGGPSSGAPPPSK

SEQ ID NO: 394 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDKRRAKAFVQWLIAGGPSSGAPPPSK

SEQ ID NO: 395 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKAFVQWLIAGGPSSGAPPPSK

SEQ ID NO: 396 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDARRAKAFVQWLIAGGPSSGAPPPSK

SEQ ID NO: 397 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDKRRAKAFVQWLIAGGPSSGAPPPSK

SEQ ID NO: 398 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYAKYLDARRAKAFVQWLIAGGPSSGAPPPSK

SEQ ID NO: 399 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDARRAKAFVQWLIAGGPSSGAPPPSK

SEQ ID NO: 400 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDKRRAKAFVQWLIAGGPSSGAPPPSK

SEQ ID NO: 401 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYAKYLDARRAKAFVQWLIAGGPSSGAPPPSK

SEQ ID NO: 402 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDARRAKAFVQWLIAGGPSSGAPPPSK

SEQ ID NO: 403 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDKRRAKAFVQWLIAGGPSSGAPPPSK

SEQ ID NO: 404 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDLSKQLESKAAQDFIEWLKAGGPSSGAPPPSK

SEQ ID NO: 405 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYAKYLDARRAKEFIEWLKAGGPSSGAPPPSK

SEQ ID NO: 406 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYSKYLDARRAKEFIEWLKAGGPSSGAPPPSK

SEQ ID NO: 407 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYAKYLDSRRAKEFIEWLKAGGPSSGAPPPSK

SEQ ID NO: 408 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYSKYLDSRRAKEFIEWLKAGGPSSGAPPPSK

SEQ ID NO: 409 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDSRRAKEFIEWLKAGGPSSGAPPPSK

SEQ ID NO: 410 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEWLKAGGPSSGAPPPSK

SEQ ID NO: 411 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDLSKQLESKAAQDFIAWLVNGGPSSGAPPPSK

SEQ ID NO: 412 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKDFIEWLKAGGPSSGAPPPSK

SEQ ID NO: 413 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAQEFIEWLKAGGPSSGAPPPSK

SEQ ID NO: 414 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDSRAAKEFIEWLKAGGPSSGAPPPSK

SEQ ID NO: 415 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDSKRAKEFIEWLKAGGPSSGAPPPSK

SEQ ID NO: 416 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLEARRAKEFIEWLKAGGPSSGAPPPSK

SEQ ID NO: 417 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKQLEARRAKEFIEWLKAGGPSSGAPPPSK

SEQ ID NO: 418 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFIEYLLEGGPSSGAPPPSK

SEQ ID NO: 419 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFVQWLLDGGPSSGAPPPSK

SEQ ID NO: 420 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFIEYLLEGGPSSGAPPPSK

SEQ ID NO: 421 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQWLLDGGPSSGAPPPSK

SEQ ID NO: 422 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEYLLEGGPSSGAPPPSK

SEQ ID NO: 423 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQWLLDGGPSSGAPPPSK

SEQ ID NO: 424 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQWLLDGGPSSGAPPPSK

SEQ ID NO: 425 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQWLLDGGPSSGAPPPSK

SEQ ID NO: 426 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEYLLEGGPSSGAPPPSK

SEQ ID NO: 427 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFIEYLLDGGPSSGAPPPSK

SEQ ID NO: 428 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFVQWLLEGGPSSGAPPPSK

SEQ ID NO: 429 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFIEYLLDGGPSSGAPPPSK

SEQ ID NO: 430 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQWLLEGGPSSGAPPPSK

SEQ ID NO: 431 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEYLLDGGPSSGAPPPSK

SEQ ID NO: 432 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQWLLEGGPSSGAPPPSK

SEQ ID NO: 433 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFIEYLLDGGPSSGAPPPSK

SEQ ID NO: 434 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQWLLEGGPSSGAPPPSK

SEQ ID NO: 435 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQWLLEGGPSSGAPPPSK

SEQ ID NO: 436 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEYLLDGGPSSGAPPPSK

SEQ ID NO: 437 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEYLLDGGPSSGAPPPSK

SEQ ID NO: 438 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFIEWLLEGGPSSGAPPPSK

SEQ ID NO: 439 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFVQYLLDGGPSSGAPPPSK

SEQ ID NO: 440 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFIEWLLEGGPSSGAPPPSK

SEQ ID NO: 441 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQYLLDGGPSSGAPPPSK

SEQ ID NO: 442 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEWLLEGGPSSGAPPPSK

SEQ ID NO: 443 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQYLLDGGPSSGAPPPSK

SEQ ID NO: 444 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFIEWLLEGGPSSGAPPPSK

SEQ ID NO: 445 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQYLLDGGPSSGAPPPSK

SEQ ID NO: 446 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQYLLDGGPSSGAPPPSK

SEQ ID NO: 447 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEWLLEGGPSSGAPPPSK

SEQ ID NO: 448 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEWLLEGGPSSGAPPPSK

SEQ ID NO: 449 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFVQYLLEGGPSSGAPPPSK

SEQ ID NO: 450 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFIEWLLDGGPSSGAPPPSK

SEQ ID NO: 451 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFVQYLLEGGPSSGAPPPSK

SEQ ID NO: 452 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEWLLDGGPSSGAPPPSK

SEQ ID NO: 453 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQYLLEGGPSSGAPPPSK

SEQ ID NO: 454 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFIEWLLDGGPSSGAPPPSK

SEQ ID NO: 455 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQYLLEGGPSSGAPPPSK

SEQ ID NO: 456 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEWLLDGGPSSGAPPPSK

SEQ ID NO: 457 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEWLLDGGPSSGAPPPSK

SEQ ID NO: 458 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQYLLEGGPSSGAPPPSK

SEQ ID NO: 459 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQYLLEGGPSSGAPPPSK

SEQ ID NO: 460 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFVQWLVNGGPSSGAPPPSK

SEQ ID NO: 461 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAWLLDGGPSSGAPPPSK

SEQ ID NO: 462 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIEYLVNGGPSSGAPPPSK

SEQ ID NO: 463 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAYLLEGGPSSGAPPPSK

SEQ ID NO: 464 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIEWLVNGGPSSGAPPPSK

SEQ ID NO: 465 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFVQWLKAGGPSSGAPPPSK

SEQ ID NO: 466 - protein, 40 aa, synthetic construct
  SITE 2: note = D-alanine
  MOD_RES 40: note = AminoEthoxyEthoxyAcetic Acyl-AminoEthoxyEthoxyAcetic Acyl-MaleimidoPropionic Acyl
YAEGTFTSDYAIYLDAQAQQDFVQWLLAGGPSSGAPPPSK

SEQ ID NO: 467 - protein, 40 aa, synthetic construct
  SITE 2: note = D-alanine
  MOD_RES 40: note = AminoEthoxyEthoxyAcetic Acyl-AminoEthoxyEthoxyAcetic Acyl-MaleimidoPropionic Acyl
YAEGTFTSDYSIYLDKIAQQDFVQWLLAGGPSSGAPPPSK

SEQ ID NO: 468 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQDFVQWLLDGGPSSGAPPPSK

SEQ ID NO: 469 - protein, 40 aa, synthetic construct; identical to SEQ ID NO: 355
  MOD_RES 2: note = Aib
  MOD_RES 40: note = AminoEthoxyEthoxyAcetic Acyl-AminoEthoxyEthoxyAcetic Acyl-MaleimidoPropionic Acyl

SEQ ID NO: 470 - protein, 40 aa, synthetic construct; identical to SEQ ID NO: 467
  SITE 2: note = D-alanine
  MOD_RES 40: note = AminoEthoxyEthoxyAcetic Acyl-MaleimidoPropionic Acyl

SEQ ID NO: 471 - protein, 39 aa, synthetic construct; identical to SEQ ID NO: 125
  MOD_RES 2: note = Aib
  MOD_RES 13: note = Aib
  MOD_RES 20: note = AminoEthoxyEthoxyAcetic Acyl-AminoEthoxyEthoxyAcetic Acyl-MaleimidoPropionic Acyl